SEQUENCE LISTING (6 sequences)

SEQ ID NO: 1 - protein, 11 aa, Homo sapiens
  MISC_FEATURE (5)..(5): where X = V, M or R
RPVAXVXPXXR

SEQ ID NO: 2 - protein, 5 aa, Homo sapiens
VANSV

SEQ ID NO: 3 - protein, 7 aa, Homo sapiens
  MISC_FEATURE (2)..(2): where X = V, M or R
PXAXVXP

SEQ ID NO: 4 - protein, 23 aa, Homo sapiens
  PEPTIDE (1)..(23): Biotinylated eptide fragment of human plectin corresponding to amino acid residues 95-117
ASLQRVRRPVAMVMPARRTPHVQ

SEQ ID NO: 5 - protein, 23 aa, Homo sapiens
QQERTKLRPVAMVRPTVRVQPQL

SEQ ID NO: 6 - protein, 3 aa, Homo sapiens
  MISC_FEATURE (2)..(2): where X = V, M or R
VXP

(19) United States
(12) Patent Application Publication
(10) Pub. No.: US 2005/0079503 A1
(43) Pub. Date: Apr. 14, 2005

(54) Binding domain of Siah (seven in absentia homolog) protein

(75) Inventors: David Douglas Lawrence Bowtell, Kew (AU); Colin Maxwell House, Essendon (AU); Michael William Parker, Newport (AU); Galina Polekhina, St Kilda (AU)

Correspondence Address: SUGHRUE MION, PLLC, 2100 PENNSYLVANIA AVENUE, N.W., SUITE 800, WASHINGTON, DC, 20037, US

(73) Assignee: PETER MacCALLUM CANCER INSTITUTE, ST. VINCENT'S INSTITUTE OF MEDICAL RESEARCH

(21) Appl. No.: 10/634,922

(22) Filed: Aug. 6, 2003

Related U.S. Application Data
(63) Continuation of application No. PCT/AU02/00118, filed on Feb. 6, 2002.

(30) Foreign Application Priority Data
Feb. 6, 2001 (AU) PR2908

(51) Int. Cl. C12Q 1/68; C07H 21/04; C12N 9/64

(52) U.S. Cl. 435/6; 435/69.1; 435/226; 435/320.1; 435/325; 536/23.2

(57) ABSTRACT

The present invention provides a binding domain of the protein Siah, which is known to be an important protein in the ubiquination pathway. The domain is capable of binding substances, co-factors and interactors of the Siah protein. The invention also provides a zinc-binding and a dimerisation domain of Siah. Also provided are methods of screening compounds that are capable of interacting with the binding domain, as well as methods of treating diseases including cancer, infertility, and inflammation.

The present invention relates to a binding domain of Siah (seven in absentia homolog) protein, and agonists and antagonists of the domain. The present invention further relates to methods for identifying agonists and antagonists and uses in therapy, prevention and diagnosis of disease.

### BACKGROUND OF THE INVENTION

Degradation of proteins is an important process, both in maintenance and signal transduction in mammalian cells. Degradation of some proteins is well understood and is a multi-step process involving recognition of a degradation signal, modification of the target protein by addition of a ubiquitin moiety, then degradation of the polyubiquitinated protein at the proteasome. Whilst the mechanism of recognition of polyubiquitin chains and proteolysis by the proteasome is a common feature and reasonably well understood, the recognition of targets and the formation of complexes to facilitate ubiquitination is only beginning to be elucidated.

The signal for protein degradation by the proteasome complex is the presence of polyubiquitin chains on targeted proteins. The ubiquitination of proteins is a multi-step process, whereby the ubiquitin is transferred via intermediate proteins to be finally ligated, via an isopeptide bond, onto lysines on the targeted protein. The first step is the ‘activation’ of ubiquitin by an enzyme known as the ubiquitin activating enzyme E1, or Uba1. The second step in the process is the transfer of the ubiquitin to a ubiquitin conjugating enzyme E2, also known as Ubc. There are at least a dozen of these proteins in mammalian systems. These first two steps involve the formation of high energy thioester bonds between ubiquitin and cysteine residues on the E1 and E2 molecules. The third step in this mechanism is the most diverse and involves proteins and complexes termed E3's, which are able to form a scaffold between the conjugated E2's Ubc's and the target proteins, thus facilitating the transfer of ubiquitin from the E2 to the target protein. It is the E3's which recognize target proteins or signals thereon, and are thus the major sites of regulation. For some proteins, the target signal is known and the E3 complex which recognizes it is well understood eg. cyclin B degradation by the anaphase promoting complex APC. For the majority of proteins, though, these processes are not understood.

Siah is the mammalian homolog of the Drosophila protein SINA seven in absentia, a protein involved in the specification of cells in the developing eye. SINA-like proteins are found in all eukaryotes except yeast, with high conservation throughout the majority of the protein. Known human isoforms of Siah are Siah1 and Siah2, with mice exhibiting forms known as Siah1a, Siah1b and Siah2. It has been shown that the mouse and human forms of Siah have high sequence identities at the amino acid level.

Tissue expression studies in mouse and human have been reported. In the mouse, Siah1 and Siah2 have been shown to be widely expressed at low levels in the embryo and adult. Northern analysis showed Siah1 to be expressed most highly in the brain, testis, thymus and lung, whereas Siah2 was most highly expressed in the brain and thymus with much lower expression elsewhere. Analysis of Siah2 by hybridization histochemistry showed that it was expressed at a restricted number of sites during development, including the olfactory epithelium, retina, forebrain and proliferating cartilage of developing bone. A study of Siah2 expression in the mouse ovary and testis showed that in both tissues the expression was confined to a specific population of developing germ cells and was undetectable in somatic cells. In a study of Siah distribution in human tissues, Siah1 was expressed highly in the placenta, testis and ovary, with less expression in heart, brain, skeletal muscle, thymus, prostate and small and large intestine. Siah2 was also expressed most highly in the placenta, with the spleen, thymus, prostate and ovary showing less expression.

The sub-cellular localization of Siah has been investigated by a number of laboratories. SINA was originally reported as being predominantly nuclear. Human Siah2 is reported as localized to the cytoplasm in Jurkat cells, with pronounced perinuclear staining. In transfection experiments Siah1a localized predominantly to the nucleus of GM701 cells, but in the cytoplasm of CHO cells. Hence the subcellular localization of the endogenous forms of both Siah1 and Siah2 is different in various cell types and under different stimuli.

Siah is involved in the degradation of a number of proteins, though the basis of the recognition is unknown. In the fly Drosophila melanogaster, the homolog SINA was shown by yeast two-hybrid assay to interact with the proteins phyllopod (PHYL) and tramtrack88 (TTK88) and to function in the degradation of the TTK88 protein. Both SINA and Siah were shown, in vitro, to promote the degradation of the mammalian protein deleted in colorectal cancer (DCC), a neuronal pathfinding molecule, and Siah promoted the in vitro degradation of the nuclear receptor co-repressor, N—CoR. More recently, Siah has been shown to bind to and degrade the motor protein Kid, the oncoprotein Myb, the anti-apoptopic protein Bag-1, the transcriptional co-activator OBF-1, the cell fate regulator numb, and the synaptic vesicle protein synaptophysin. In other work, Siah has been shown to interact with other proteins, such as Vav, mGlutR, PW1, α-tubulin, SIP and APC though not necessarily to promote their degradation. The role of co-factors is not completely understood at present. It is not known whether these co-factors are simply part of a scaffold or whether they contribute to the enzymatic mechanism via allosteric activation or the like.

Details of the Siah domain to which binding partners may interact have not yet been provided by the prior art. Accordingly, Applicants have now crystallised and determined the structure of the Siah protein by X-Ray crystallography. This structure will allow for the rational design of agonists and antagonists which may be useful for therapy, prevention and diagnosis of disease.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed in Australia before the priority date of each claim of this application.

### SUMMARY OF THE INVENTION

The present invention provides a binding domain or portion thereof of a Siah protein comprising amino acid residues found in the cysteine rich region (residues 98 to 152) and/or the C-terminal region (residues 152 to 282) of Siah1a or equivalent residues in other Siah family proteins. The binding domain has been shown to be capable of binding to a protein selected from the group consisting of DCC, PHYL, N—CoR, mGlutR1, PW-1, Kid, α-tubulin, SIP, OBF-1, Numb, pAPC, synaptophysin and Vav.

Applicants have been successful in crystallizing the Siah protein, and have resolved the X-Ray structure. The Siah binding domain is known to be involved in the ubiquitin-mediated protein degradation pathway, and the provision of agonists and antagonists to the domain will be useful in the treatment of many diseases. Also provided by the present invention is a dimerisation domain or portion thereof of a Siah protein. Applicants have demonstrated that the binding domain of Siah may be formed upon dimerisation of two Siah monomers.

Determination of the X-Ray structure of Siah has also identified a number of Zinc binding domains. Amino acid residues shown to be involved in the co-ordination of zinc include Cys128, Cys135, His147, His152, His150, Cys98, Cys105, His117 and Cys121, His230, Glu269, His230, and Glu269.

Also provided by the present invention is a ligand capable of binding to a binding domain comprising the amino acid sequence RPVAXVXPXXR.

The present invention also provides a method of identifying a compound which is capable of acting as an agonist or antagonist of binding to Siah, the method comprising the step of identifying a compound that has a conformation and polarity such that it interacts with a relevant amino acid residue.

Also provided is a computer-assisted method of identifying compounds potentially able to bind to the binding domain of Siah using a programmed computer the method comprising the steps of:
(a) inputting into the programmed computer data comprising the atomic coordinates of the Siah binding domain, corresponding to a binding site defined by a relevant amino acid residue
(b) generating, using computer methods, a set of atomic coordinates of a structure that possesses stereochemical complementarity to the atomic coordinates defined in (a) or a subset thereof, thereby generating a criteria data set;
(c) comparing, using the processor, the criteria data set to a computer database of chemical structures;
(d) selecting from the database, using computer methods, chemical structures which are similar to a portion of the criteria data set; and
(e) outputting the selected chemical structures which are similar to a portion of the criteria data set. Also provided is a computer or a software component thereof for producing a three-dimensional representation of a molecule or molecular complex, which comprises a three-dimensional representation of a homologue of the molecule or molecular complex, in which the homologue comprises a domain that has a root mean square deviation from the backbone atoms of the amino acids of not more than 1.5 Å, in which the computer comprises:
(a) a machine-readable data storage medium comprising a data storage material encoded with machine-readable data, wherein the data comprises the atomic coordinates of relevant amino acid residues
(b) a working memory for storing instructions for processing the machine-readable data;
(c) a central-processing unit coupled to the working memory and to the machine-readable data storage medium for processing the machine-readable data into the three-dimensional representation ; and
(d) a display coupled to the central-processing unit for displaying the three-dimensional representation.

The invention further provides a computer or a software component thereof for determining at least a portion of the structure coordinates corresponding to a three-dimensional structure of a molecule or molecular complex, in which the computer comprises:
(a) a machine-readable data storage medium comprising a data storage material encoded with machine-readable data, in which the data comprises at least a portion of the atomic coordinates according to FIG. 21;
(b) a machine-readable data storage medium comprising a data storage material encoded with machine-readable data, wherein the data comprise crystallographic data of the molecule or molecular complex;
(c) a working memory for storing instructions for processing the machine-readable data of (a) and (b);
(d) a central-processing unit coupled to the working memory and to the machine-readable data storage medium of (a) and (b) for performing a transformation of the machine readable data of (a) and for processing the machine-readable data of (b) into structure coordinates; and
(e) a display coupled to the central-processing unit for displaying the structure coordinates of the molecule or molecular complex.

Also provided is a compound able to act as an antagonist or agonist of the binding of protein substrate and/or co-factor and/or interactor and/or ligands to the binding domain of Siah, wherein the compound is identified by the methods disclosed herein. The invention further includes compositions including these compounds.

Also included are methods of treating or preventing a disease using the compositions described herein. The disease may be selected from a disease relating to abnormal protein degradation, cancer, inflammation, infertility, a pathological immune response, a disease relating to apoptosis, a disease relating to NFκB signalling, or a neurological disorder.

Still further provided is a method of assaying ubiquitination the method comprising the steps of providing a source of Siah, E1 enzyme, E2 enzyme, a substrate, ubiquitin, and ATP, and wherein the substrate is ubiquinated if it is capable of binding to a binding domain of Siah.

The present invention further provides is a method of assaying NFκB activation the method comprising the steps of providing a cell transfected to express Siah and measuring the activation of NFκB.

The invention yet further provides a crystalline form of Siah. Applicants have shown that in a crystalline form the Siah dimer comprises 4 molecules of 2-ME, 6 zinc ions and 65 water molecules.

The present invention still further provides a method of producing a crystal of Siah or a fragment thereof comprising using partially oxidised 2-ME.

### DESCRIPTION OF THE FIGURES

FIG. 1a illustrates the general structure of domains present in the Siah1A protein. Amino acid residue number is shown under the molecule in bold.

FIG. 1b illustrates the amino acid sequence of human Siah2 protein. The sequence of the RING structure is underlined.

FIG. 2 illustrates a sequence alignment for Siah/SINA molecules from a range of species. The black shading indicates totally conserved residues, while the grey shading indicates greater than 65% conservation ie identical or conservative changes.

FIG. 3 is a size exclusion chromatography elution profile of MBP-Siah 1a fusion protein using absorbance at 280 nm as an indicator of protein concentration. Elution times for molecular weight standards are shown.

FIG. 4a demonstrates the limited proteolysis at 30° C. of MBP-Siah1a fusion protein using a range of proteases for varying time periods analysed by SDS-PAGE. Molecular weight standards are shown on the left, with the identity of protein bands shown at the right.

FIG. 4b illustrates comparative limited proteolysis of MBP-Siah1a using 10 μg of the indicated protease and 200 μg of MBP-Siah1a. The digestion was terminated after 30 minutes at 30° C. The identity of molecular weight markers are shown to the left, and protein bands are shown to the right.

FIG. 5a shows N-terminal sequencing of stable Siah1a domain after proteolysis with trypsin and partial purification. Only the first five cycles are shown. The sequence determined for the N-terminus is VANSV.

FIG. 5b illustrates electrospray mass spectrometry of the stable Siah1a fragment that was sequenced in FIG. 5a.

FIG. 6 shows the purification of MBP-Siah1a ZC (ZC is the Siah substrate binding domain, encompassing residues 80-282 and is so called because it contains zinc fingers and the C-terminus) fusion protein using amylose affinity chromatography by SDS-PAGE. Note that the majority of protein elutes in the first two column volumes of the maltose elution.

FIG. 7 shows a chromatogram for anion exchange chromatography of the tryptic fragment from MBP-Siah1a ZC using Mono Q resin.

FIG. 8 shows a chromatogram for size exclusion chromatography of the tryptic fragment from MBP-Siah1a ZC using Superdex 200 resin.

FIG. 9 shows a chromatogram for a second chromatography step using Mono Q resin for the tryptic fragment from MBP-Siah1a ZC.

FIG. 10 shows size exclusion chromatography of the final Siah1a binding domain using Superdex 200 resin. The elution of molecular weight standards are shown.

FIG. 11 details studies on the zinc content of Siah fusion proteins.

FIG. 12 shows electrospray mass spectrometry of reduced Siah binding domain.

FIG. 13 is a graphical representation of the deduced crystal structure of Siah binding domain. The lower representation is a 90 degree rotation of the upper representation.

FIG. 14 is a topology map of Siah β-sandwich. The arrows represent β-strands, while the cylinders represent α-helices.

FIG. 15 illustrates the surface area of the dimer interface. Residues which contribute to the interaction are shaded.

FIG. 16 is a stereo-view of the overlay of the TRAF-C β-sandwich (dark lines) onto the Siah structure (light lines).

FIG. 17 shows surface diagrams of Siah1A binding domain (left hand side) and the corresponding secondary structural features (right hand side) from three different view points. Conserved residues are shown in red.

FIG. 18a is a view of the Siah binding domain. The structure is dimeric. The first zinc finger is blue, the second zinc finger in red, and the two p-sandwich structures in green and orange.

FIG. 18b is a 90 degree rotation of the view of the Siah binding domain shown in FIG. 18a.

FIG. 19 shows a comparison of the β-sandwich structures of Siah and the TRAF-C domain of the TNF-receptor associated factor.

FIG. 20 illustrates the zinc fingers of Siah1a binding domain. The first zinc finger is in blue, and the second zinc finger in red. Zinc coordinating residues, with Siah1a numbering are shown.

FIG. 21 lists the coordinates for atoms included in the Siah binding domain. These coordinates can be entered into software such as RasMol (Version 2.6, author: R. Sayle) to produce a three dimensional image. This software can be downloaded from the internet at URL http://www.umass.edu/microbio/rasmol/rasquick/html.

FIG. 22 shows the graphical output from the RasMol software when the coordinates of FIG. 21 are entered.

FIG. 23 shows PHYL108-130 binds Siah. GST-PHYL108-130 was immobilised on GSH-Sepharose, and then mixed with MBP-Siah for 1 hour at 4° C. Unbound material was washed from the resin with four washes of 50 mM Tris pH 8, 200 mM NaCl, 15 mM 2-ME. The bound material was visualised by Coomassie blue staining of SDS-PAGE of the boiled/denatured resin.

FIG. 24 shows Siah/PHYL complex on gel filtration. A mixture of Siah binding domain and GST-PHYL108-130 was subjected to gel filtration on Superdex 200 (Pharmacia). Fractions were collected and the protein content analysed by SDS-PAGE, visualised using Coomassie blue.

FIG. 25 shows Siah/PHYL complex on anion-exchange chromatography. A mixture of Siah binding domain and GST-PHYL108-130 was subjected to anion-exchange chromatography on a Mono Q column (Pharmacia). Fractions were collected and the protein content analysed by SDS-PAGE, visualised using Coomassie blue.

FIG. 26 shows binding affinity between Siah and PHYL108-130. The binding kinetics for Siah interaction with PHYL108-130 were monitored using surface plasmon resonance on a Biacore 2000. The biotinylated PHYL108-130 peptide was captured on the chip surface by immobilised neutravidin, and MBP-Siah1a was used as analyte. The affinity constant determined from these data was 180 nM.

FIG. 27 shows IC50 values for mutant peptides. Mutant peptides were used in a competition assay in which they were tested as competitors of wild-type PHYL 108-130 binding to Siah. GST-PHYL108-130 was immobilised in wells of a 96-well plate and MBP-Siah was bound for 1 hour at room temperature, in the presence of various concentrations of mutant peptides. After binding and washing, the amount of bound MBP-Siah was quantitated in a two antibody sandwich of anti-MBP antibody, followed by HRP-conjugated goat anti-rabbit antibody, using ABTS as substrate. IC50 (the concentration at which 50% inhibition is observed) values were estimated from the individual inhibition curves.

FIG. 28. shows mutant peptide competition at 300 nM. Data for 300 nM competing peptide, from the inhibition curves generated in the experiments described in FIG. 5, were used to show the effectiveness of mutating particular residues in PHYL108-130 to alanine.

FIG. 29 shows mutant peptide competition at 1000 nM. Data for 1000 nM competing peptide, from the inhibition curves generated in the experiments described in FIG. 5, were used to show the effectiveness of mutating particular residues in PHYL108-130 to alanine.

FIG. 30 shows residues mutated to opposite characteristic (300 nM peptide). Individual residues in PHYL108-130 were changed to residues with opposite characteristics and peptides were assessed in the competition binding assay outlined in Figure legend 5. Data are shown for 300 nM competing peptide.

FIG. 31 shows residues mutated to opposite characteristic (1000 nM peptide). Individual residues in PHYL108-130 were changed to residues with opposite characteristics and peptides were assessed in the competition binding assay outlined in Figure legend 5. Data are shown for 1000 nM competing peptide.

FIG. 32 shows double amino acid mutants of PHYL108-130 (300 nM peptide). Mutant peptides were synthesised in which multiple amino acids were changed to alanine. Altered residues were based on the binding motif predicted from sequence alignment (PXAXVXP). Data are shown for 300 nM competing peptide.

FIG. 33 shows double amino acid mutants of PHYL108-130 (1000 nM peptide). Mutant peptides were synthesised in which multiple amino acids were changed to alanine. Altered residues were based on the binding motif predicted from sequence alignment (PXAXVXP). Data are shown for 1000 nM competing peptide.

FIG. 34 shows Siah SBD binds to plectin peptide. Siah SBD binding to the plectin peptide (Biotin-ASLQRVRRPVAMVMPARRTPHVQ-NH2 corresponding to residues 95-117 of human plectin) was assessed by capturing biotinylated peptide on a 96-well plate coated with 0.5 μg Neutravidin per well. Peptide was captured for 30 min at room temperature at a concentration of 4 μg/ml. MBP-Siah1a-ZC fusion protein was bound to the peptide for 50 min at room temperature (or the controls of buffer alone (PBS/tween 20) or mutant MBP-Siah1a-ZC containing a Leu211 to Arg mutation). Bound protein was quantitated by rabbit anti-MBP antibody (1:300 dilution), then HRP conjugated goat-anti-rabbit antibody. Detection utilised ABTS substrate read at A405.

### DETAILED DESCRIPTION OF THE INVENTION

The present invention provides a binding domain or portion thereof of a Siah protein.

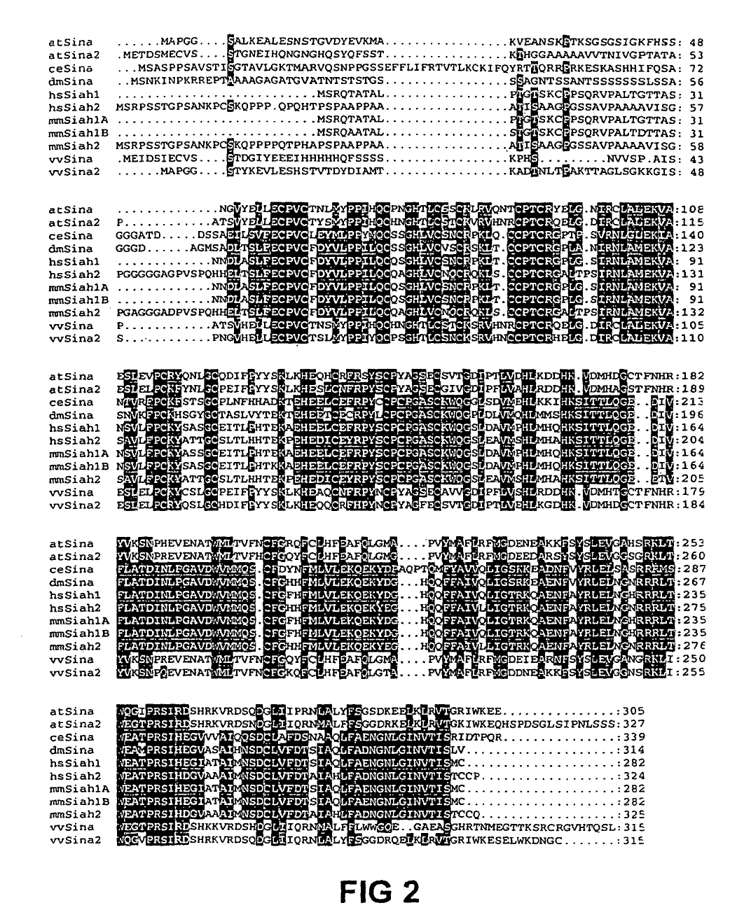

As used herein the term “Siah” includes all analogues, homologues and isoforms of Siah, including but not limited to Siah1 of humans, Siah2 of humans, Siah1a of mice, Siah1b of mice, and Siah2 of mice and SINA of Drosophila. It is likely that other members of the Siah family are yet to be identified, and it should be understood that the present invention is not limited to these presently known forms of Siah. Mouse Siah1a and 1b are highly homologous and differ by only 6 amino acids, three of which are conservative changes. The human Siah1 protein is almost identical to mouse Siah1a. Siah2, however, has a longer and divergent N-terminus whilst the rest of the molecule is highly conserved. The majority of differences between Siah1 and Siah2 are in the N-terminus and RING domain (FIG. 2).

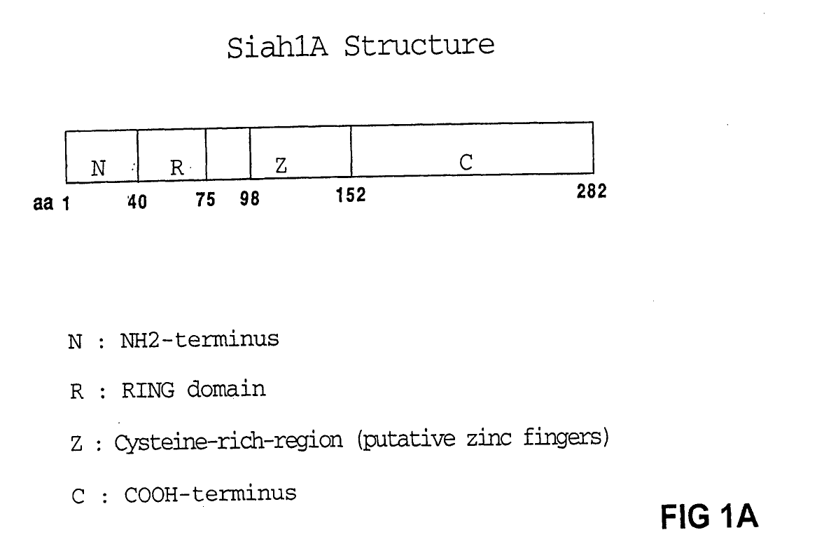

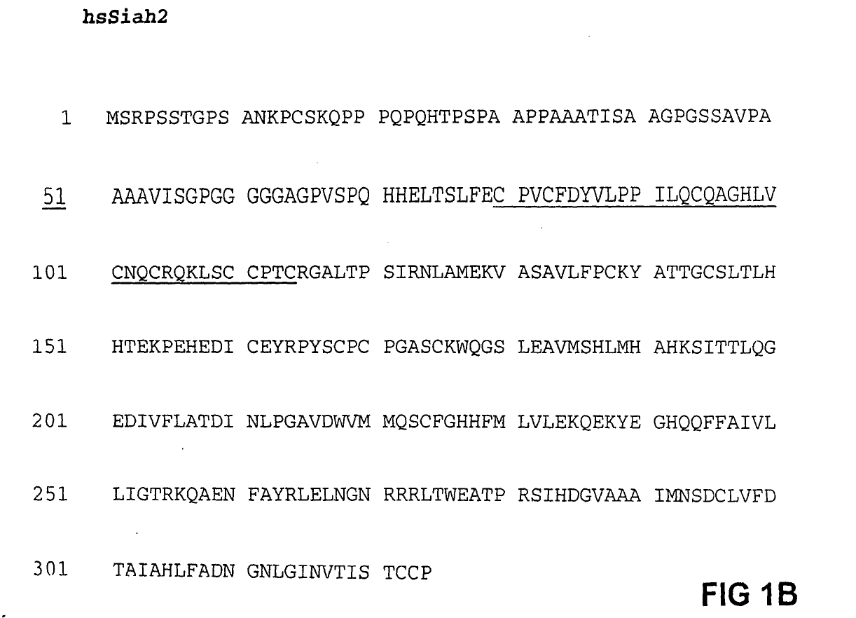

The general structure of the SINA/Siah proteins is shown in FIG. 1a, using Siah1a as an example. The proteins can be broadly assumed to consist of four regions, an N-terminal region, a RING domain, a cysteine/histidine-rich region and a C-terminal region. The majority of the protein appears unique, except for two regions. The first of these is the RING domain (FIG. 1b), a zinc-coordinating domain which occurs in a number of other proteins and has been shown to be involved in the process of ubiquitination via the binding of Ubc proteins. The second region is the cysteine/histidine-rich region just C-terminal of the RING domain, which aligns with part of a putative zinc finger region of the TRAF family of proteins. This region in TRAF2 has been shown to be important for NF-κB signalling in TRAF2 overexpression experiments (Takeuchi et al., 1996). Within the SINA/Siah proteins, the regions including and C-terminal of the RING domain are highly conserved (FIG. 2), but there is divergence at the N-terminus.

Applicants have crystallized and defined the limits of a stable domain of the protein Siah. The domain incorporates the binding region of Siah and is able to bind to previously described binding partners. It comprises a conserved cysteine- and histidine-rich region which Applicants have shown to bind two atoms of zinc per molecule. The domain is apparently dimeric by size exclusion chromatography and the deduced crystal structure, but the zinc-binding region is not involved in dimerization. The zinc binding region adopts the topology of classic DNA-binding zinc fingers, with both fingers adopting the structure β-strand, β-strand, α-helix. The rest of the molecule incorporates the dimerization domain, which assumes a β-sandwich structure similar to the TRAF-C domain of the TRAF molecules. The interacting face on the TRAF-C domain is masked with an α-helix in Siah, which apparently precludes an interaction mimicking the TRAF/TNF receptor interaction. This region of the molecule, however, may still be utilized for protein/protein interactions as it is an exposed region in the Siah crystal structure.

Preferably, the binding domain is capable of binding to a substrate and/or co-factor and/or interactor. As used herein the term “substrate” is taken to mean a protein that is capable of binding to Siah either directly or indirectly via one or more proteins that is subsequently ubiquitinated as a result of the binding. As used herein the term “co-factor” is taken to mean a protein that binds to Siah either directly or indirectly via one or more proteins, thereby facilitating the alteration of a substrate. As used herein the term “interactor” is taken to mean a protein that binds to Siah, the outcome of which is for functions other than ubiquitination of the interactor or any other protein by Siah.

In a preferred form of the invention the domain is capable of binding to a protein selected from the group consisting of DCC, PHYL, N—CoR, mGlutR1, PW-1, Kid, α-tubulin, SIP, OBF-1, Numb, synaptophysin, pAPC and Vav.

In a further preferred embodiment the domain is capable of binding to at least one protein involved in protein degradation. The term “protein involved in protein degradation” as used herein includes a protein which is an accessory protein or co-factor which helps in the degradation of another protein. The term also includes the protein itself which has been targeted for degradation.

In a more preferred form the binding domain is capable of binding a protein involved in the ubiquitination of proteins. As discussed previously, ubiquitination of a protein “labels” that protein for degradation by other enzymes.

Applicants have demonstrated the binding capacity of the Siah1a C-terminus using fragments of the human DCC protein as well as fragments of the Drosophila protein PHYL, which has previously been shown to interact with the SINA protein in yeast two-hybrid assays (Tang et al., 1997). The fragment was stable for short periods (5-10 min at 0° C.) to trypsin concentrations 10-fold higher than those used in the initial characterization 1:10, despite the presence of 14 basic residues arginine or lysine which could serve as potential sites of tryptic cleavage within the protein. This stability suggested that the fragment was a potential candidate for crystallization trials, where a lack of mobility and flexibility within a protein appears to be crucial.

Proteolytic stable fragments of a protein can be determined by a time course of protease digestion with a set amount of protease, or similarly digestion for a single time using different amounts of protease. Fragments can be monitored by mass spectrometry or SDS polyacrylamide gel electrophoresis. Stable fragments persist for long periods in the time course, or in the presence of excessive amounts of protease.

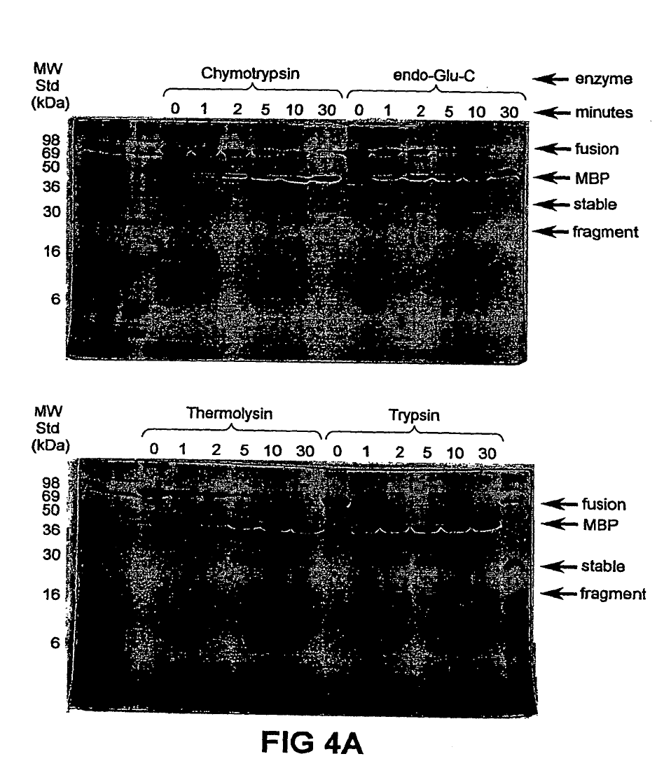

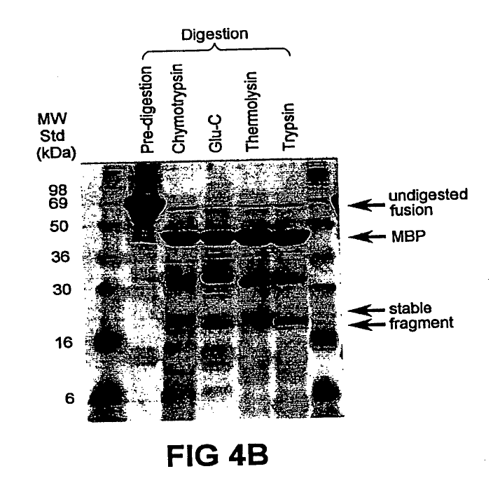

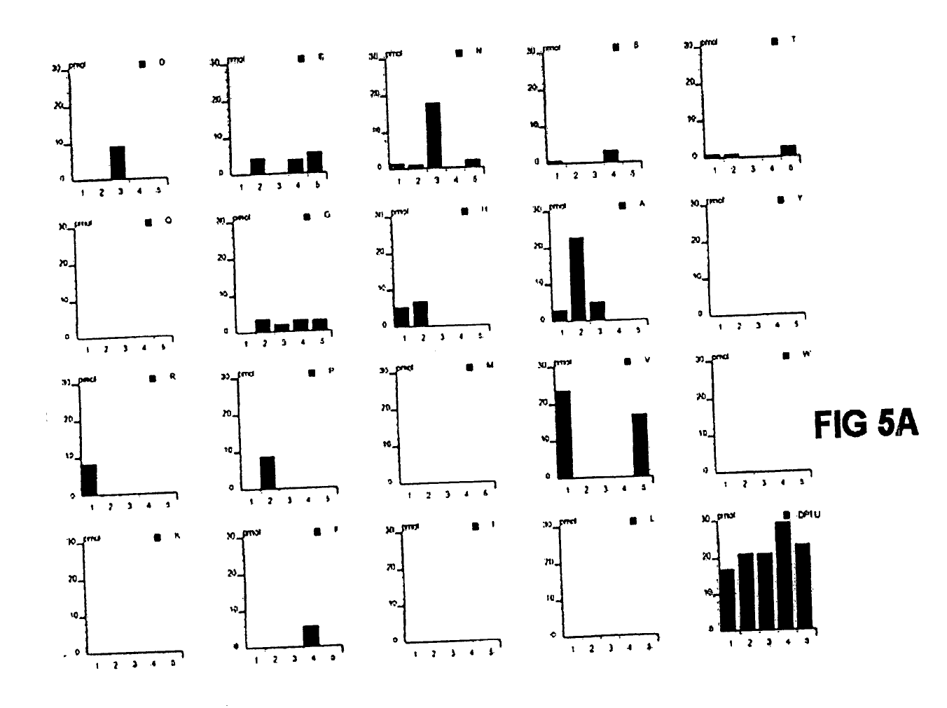

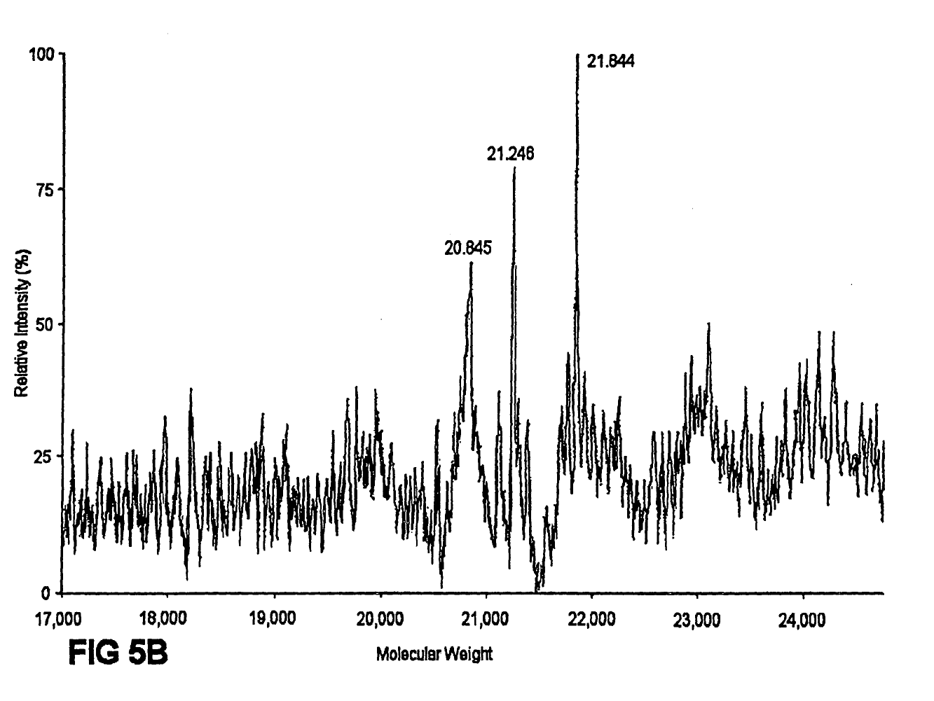

To identify stable fragments, in a preferred example MBP-Siah1a fusion protein was subjected to limited proteolysis by a number of proteases including trypsin, chymotrypsin, thermolysin and Glu-C at 1:100 protease to protein ratio. These proteases may generate stable fragments of approximately 21.5-23 kDa (FIG. 4a and 4b). Characterization of the tryptic fragment by N-terminal sequencing and electrospray mass spectrometry defined a fragment consisting of residues spanning the region from just C-terminal of the RING domain to the C-terminus (FIG. 5a and 5b). This fragment encompasses the interaction domain as described for the proteins DCC (Hu and Fearon, 1999), N—CoR (Zhang et al., 1998), mGlutR1 (Ishikawa et al., 1999), PW-1 (Relaix et al., 2000), Kid, α-tubulin (Germani et al., 2000) and Vav (Germani et al., 1999).

In a preferred aspect of the invention, the binding domain comprises amino acid residues found in the cysteine rich region (residues 98 to 152) and/or the C-terminal region (residues 152 to 282) of Siah1a or equivalent residues.

In a preferred form of the invention, the binding domain comprises amino acid residues found in the region comprising amino acid residues 144 and 177 of Siah1a or equivalent residues.

More preferably, the region comprises a β-strand, β-strand, α-helix structure. Applicants have discovered that this structure defines a “groove” within which there is a pocket which is occupied by β-ME in the crystal structure. This pocket is therefore possibly occupied under normal physiological circumstances, possibly by part of a binding partner.

More preferably, the domain comprises a region adjacent the helix α2 comprising amino acid residues Ile247 to Met252. This region can be considered as a shallow “groove” which is located along one side of the helix.

Preferably, the domain comprises a Lysine residue which is capable of binding with a region on a substrate and/or co-factor and or interactor to allow recognition and/or binding of the substrate and/or co-factor and/or interactor to the domain. More preferably the Lysine is Lys114 and/or Lys153 of Siah1a or equivalent residues.

In another preferred aspect, the binding domain further comprises a glutamic acid residue which is capable of binding with a region on a substrate and/or co-factor and/or interactor to allow binding of the substrate and/or co-factor and/or interactor to the domain. In a more preferred embodiment, the glutamic acid is Glu226 of Siah1a or equivalent residue.

In another preferred aspect, the binding domain or portion thereof capable of binding the substrate and/or co-factor and/or interactor may include the residues defined by Arg241 monomer A, Glu255 monomer A, and Arg232 monomer B.

In another form of the present invention, the binding-domain or portion thereof capable of binding the substrate and/or co-factor and/or interactor may include the residues Gly132, Glu194, and Trp178. On the other side of helix α2 there is a “valley” with the base formed by the face of the β-sheet made up from β3, β4, β5 and the sides formed by the loop connecting β1 and β2 of the second zinc finger domain and the β-hairpin of β4β5. Strictly conserved Gly132, Glu194, Trp178 are located in the base of this valley. Gly132 and Glu194 make up about 70 Å² of accessible surface.

In another preferred aspect, the binding domain or portion thereof capable of binding the substrate and/or co-factor and/or interactor may be defined by the residues Gly160, Cys184, Phe185, Phe187, Phe221, Met281 and Gly186. Strictly conserved Gly160, Cys184 and Phe185 and Phe187, Phe221, Met281 and Gly186 form a significant hydrophobic patch with the accessible area of about 366 Å² which is located on the edge of the concave surface of the molecule created by the continuous antiparallel eight β-strand sheet. This patch is in the vicinity of helix al of the second zinc binding domain. Glu226 is the only strictly conserved and surface exposed about 20 Å² of accessible area residue which is located in the concave surface of the molecule.

Siah Structure Determination by X-ray Crystallography.

To understand the basis of Siah substrate, co-factor or interactor recognition, how it may be regulated and to predict unidentified substrates, it was necessary to crystallize Siah in the absence and presence of substrate. Previous work with full-length Siah protein was problematic, apparently because of the presence of the RING domain (FIG. 1b). The RING domain occurs in hundreds of proteins and has proven difficult for many workers to express and fold properly (Borden and Freemont, 1996). Published data suggest that the binding domain of Siah resides in the C-terminal portion of the molecule (DCC, Kid, N—CoR, OBF-1, Numb and synaptophysin) distinct from the N-terminus and RING domain, which appear to interact with the ubiquitin conjugating enzymes, Ubc's.

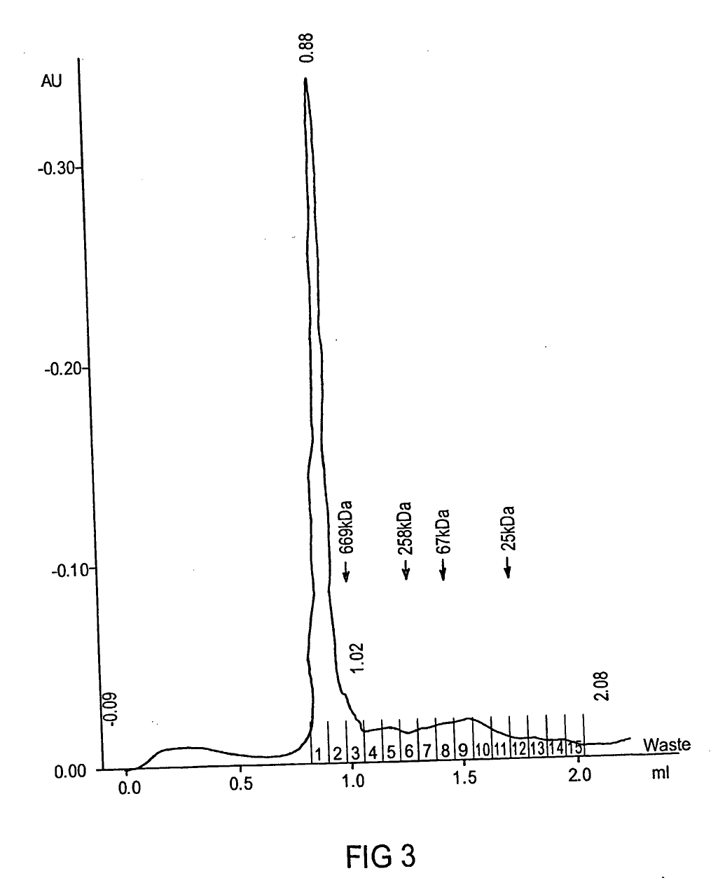

Initial attempts to purify Siah fragments as fusion partners of glutathione-S-transferase GST were unsuccessful due to protein aggregation. It was presumed that this was due to the dimerization of both Siah and GST, giving rise to polymers which did not fold properly and/or aggregated and were thus insoluble. We have found this to be true of all constructs which include the dimerization domain of Siah. The maltose-binding protein MBP fusion partner does not dimerize, so was considered to be a viable alternative for expression and purification by affinity chromatography. Siah1a was cloned into the pMalC2 plasmid at the BamHI and HindIII sites. Whilst the fusion protein was soluble, it was apparently aggregated, as assessed by size exclusion chromatography (FIG. 3).

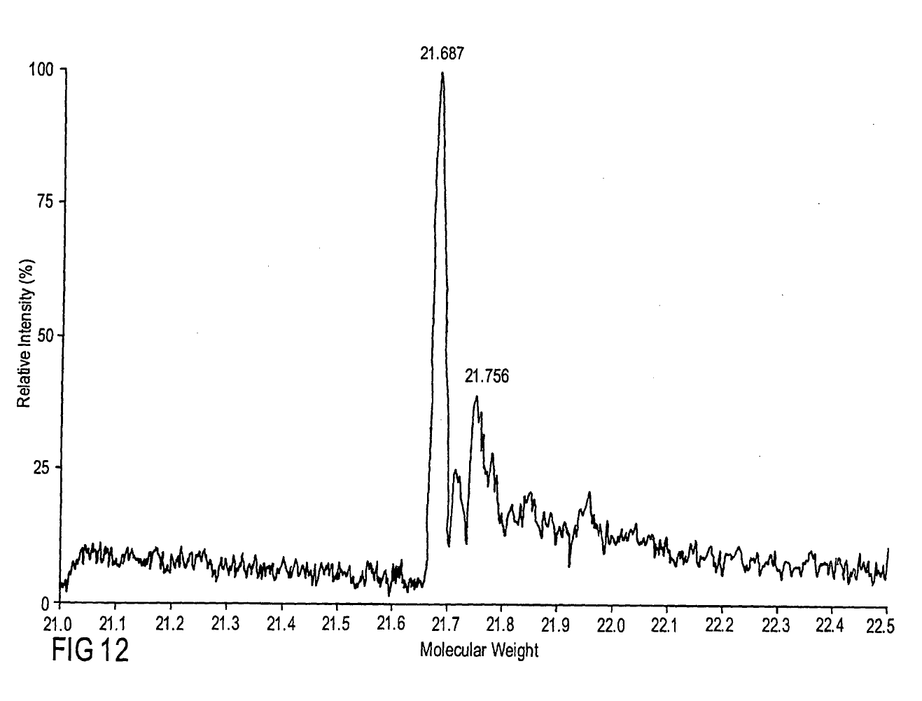

Mass spectrometric analysis of purified Siah binding domain revealed that the protein was predominantly in a form containing two bound 2-mercaptoethanol (2-ME) adducts (FIG. 4b). These were presumably bound to cysteine residues as they could be reduced off the protein with fresh dithiothreitol to generate a protein of the predicted mass 21,685 Da (FIG. 12) as determined by electrospray mass spectrometry. Subsequent mass spectrometric analysis of protein crystals also revealed that they consisted of the ‘oxidized’ form 21,838 Da. Batches of protein purified in the presence of fresh 2-ME failed to crystallize, and only when ‘older’ 2-ME was used could crystals be obtained. It was concluded that crystallization of the protein required two of the cysteines to be oxidized to 2-ME, perhaps to aid in crystal contact formation by stabilization of flexible regions within the structure. Subsequently, the presence of these 2-ME adducts was confirmed in the crystal structure.

It appears that the use of partially oxidized 2-ME is important for the crystallization of the Siah fragment under the conditions used. Although serendipitous, this is an important observation. Whilst one would expect this oxidation to occur with time, as the reducing agent 2-ME gradually oxidized, this doesn't seem to be the case with the Siah fragment. The protein is prone to precipitation in the crystallization conditions, and this increases with time, so that unless nucleation and crystallization occur in the first week or two, then the protein concentration diminishes to a point where crystallization is unlikely to occur.

The initial characterisation of Siah1 crystals may be performed on an in-house X-ray source. X-ray crystallography relies on the observation that if a parallel X-ray beam is passed through a molecule, the X-rays will be deflected by electron dense regions. The scattering of the parallel X-ray beam will give a diagnostic deflection pattern, depending on the structure of the molecule. Unfortunately, molecules in solution are mobile and not aligned with their neighbouring molecules, meaning that the X-ray diffraction will be diffuse and non-interpretable. If, however, all of the molecules of a similar type are aligned in an orderly fashion for example, in a crystal, then X-ray diffraction will be orderly and the pattern of diffraction contains structural information about the molecule of interest. In X-ray crystallography, a crystal of, say, protein is bombarded with X-rays whilst it is rotated through an angle of 90°, thus allowing a ‘data set’ to be collected. Data is collected on an image plate or photographic film and interpreted by computer software because of the enormous number of data point and intensities collected. The three dimensional structure of the protein can be determined in this manner.

Siah1 crystals showed diffraction to about 2.5 Å. They were sensitive to radiation damage, therefore the data collection had to be done at cryogenic temperature 100K. 15% of MPD was added to the stabilizing solution of 100 mM MES pH 6.5, 25% ethanol for cryo-protection. Siah1 crystals belong to space group P2₁2₁2, with the cell parameters a=65.9 Å, b=73.6 Å, c=80.3 Å. The unit cell dimensions were consistent with a dimer in the asymmetric unit corresponding to about 40% solvent content. The expected non-crystallographic two-fold axis could not be located in the self-rotation function. It was subsequently found that the non-crystallographic axis is nearly parallel to a crystallographic a* axis. Applicants had limited success with the search for heavy atom derivatives. A platinum derivative was prepared, but it did not produce sufficiently good phases on its own. It was also attempted to replace zinc ions with different lanthanides by either soaking them into crystals or by co-crystallization. Lanthanide soaks lead to change in the cell parameters of up to 2 Å for the shortest axis prohibiting their use due to non-isomorphism. The structure was therefore determined using the anomalous scattering of the bound zinc ions. Complete anomalous data sets at the zinc absorption maximum λ₁=1.28283 Å, at the zinc absorption edge λ₂=1.28336 Å, and at two remote wavelengths λ₃=1.24421 Å and λ₄=1.28616 Å were collected on the beam line of 14-BM-D at the Advanced Photon Source. Fluorescence energy scans of Siah crystals were recorded in order to precisely determine the absorption edge and the absorption maximum. Data were processed with DENZO/SCALEPACK. Zinc positions were determined from the anomalous Patterson maps calculated between 30 and 4.5 Å resolution. The positions of 4 zinc ions were refined first using the program VECREF. Further refinement and final phases were calculated with the program MLPHARE. Both enantiomorphs were taken into account, and solvent flattening was performed using the program DM. The secondary structure elements such as β strands could be seen in the electron density maps corresponding to one enantiomorph, while the maps for the other did not display any protein features. The maps were of good quality, and allowed unambiguous interpretation.

After a partial model consisting of residues 124-282 was built it was realized that two of the four zinc ions were located not in the putative zinc fingers but in unexpected positions. One zinc ion was found in the dimer interface and an another one was coordinated by two symmetry related molecules. Two other zinc ions were located in the second zinc finger domains of each monomer. An anomalous Fourier map was therefore calculated using the phases from the current model and identified two extra peaks corresponding to two zinc ions in the positions where the first zinc finger domains-were likely located. Initially no zinc was added to the crystallization drops. However, it was later discovered that addition of 50 μM zinc acetate to the crystallization conditions improved the electron density in the first zinc finger domain and finally allowed Applicants to model the first zinc binding domain residues 93-123. The refinement was performed with the program CNS. Bulk solvent correction and restrained isotropic individual B-factors were refined with the restraints. The data collection and refinement statistics are given in Table 1.

The model of the Siah1 dimer comprises residues from 93 to 282 of both monomers, 4 molecules of β-mercaptoethanol, 6 zinc ions and 65 water molecules and has been refined to a crystallographic R-factor of 22% R-free of 27.7% to 2.6 Å resolution against the data set collected in-house. The stereochemical quality of the final model, and other stereochemical parameters such as side chain chi angle values, peptide bond planarity, α-carbon tetrahedral distortions and non-bonded interactions are all better than the allowed ranges according to PROCHECK.

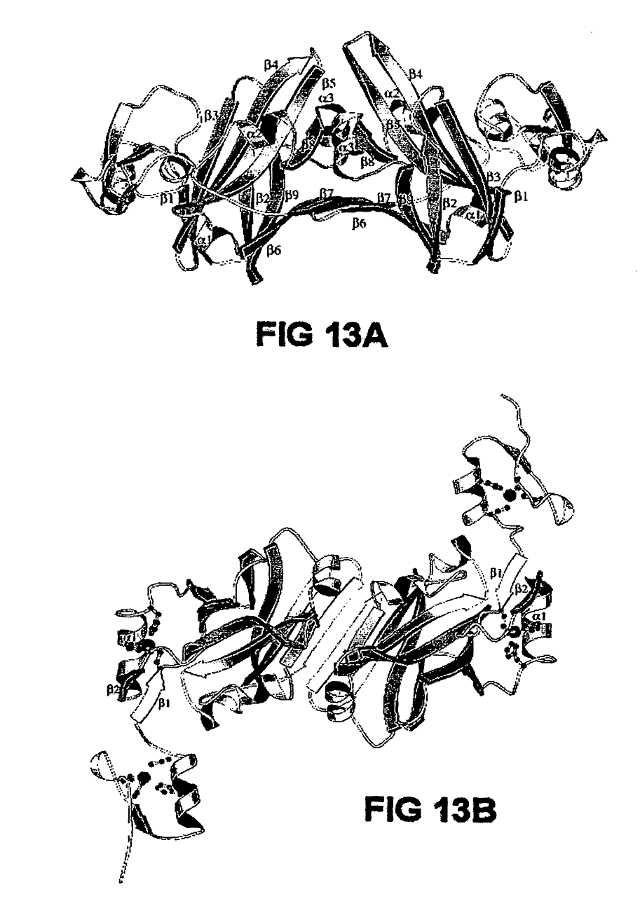

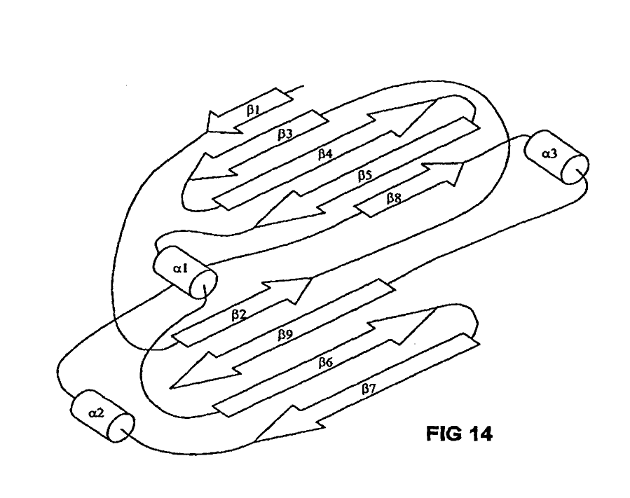

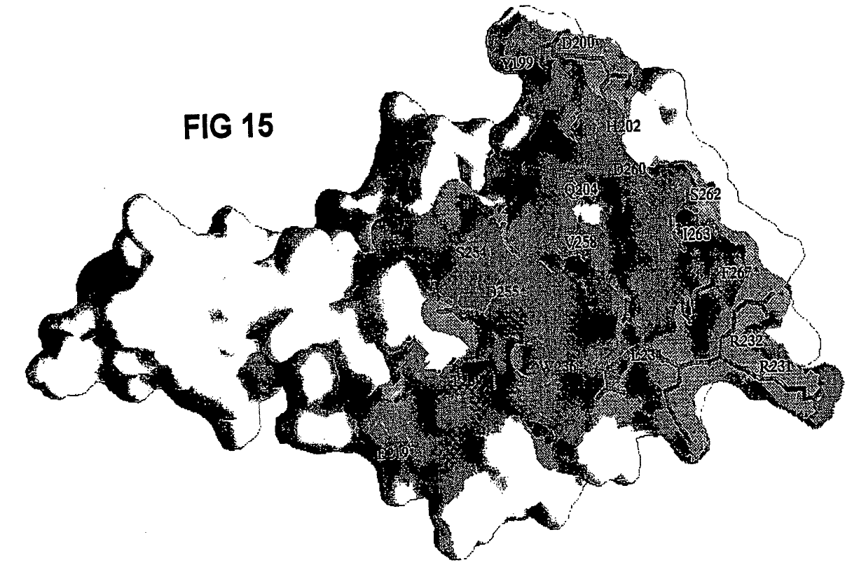

The structure presented here contains two zinc finger domains and the C-terminal domain of Siah1 (FIG. 13). Siah1 dimer, when viewed down the two-fold non-crystallographic axis, adopts an S-shaped structure (FIG. 13). At the dimer interface the C-terminal domains of the two monomers are tightly associated with each other and the zinc finger domains are at each tip of the molecule. The C-terminal domain corresponding to residues 155-282 of the whole Siah1 molecule is folded into an eight-stranded β-sandwich, with strands β3, β4, β5 and β8 in one sheet and β2, β9, β6 and β7 in the other. An additional strand, β1, runs parallel to strand β3 and connects the zinc finger domains to the C-terminal domain. The connections between β5 and β6, β7 and β8, β8 and β9 contain short helices. The topological diagram of the C-terminal domain of Siah is shown in FIG. 14. An automatic structural similarity search using the DAL1 program identified TRAF tumour necrosis factor TNF-receptor-associated factors as the top hit. The visual inspection confirmed that Siah and TRAF do indeed share the same topology. TRAFs are adapter proteins that mediate signal transduction from many members of the TNF-receptor superfamily. Interestingly, TRAF and Siah proteins also share a similar organization: RING finger domain, followed by a series of zinc finger domains and a C-terminal domain involved in protein-protein interactions. The sequence identity in their N-terminal part has been noted previously (Matsuzawa et al., 1998). However, no sequence similarity can be detected in the C-terminal part. Despite the structural similarity of the C-terminal domains of Siah and TRAF, their mode of self-association is totally different. Siah exists as a dimer, while TRAF is a trimer and requires at least three heptad repeats from the TRAF-specific coiled-coil domain preceding its C-terminal domain for trimer formation.

Both zinc fingers adopt the topology of the classical zinc finger, β-strand, β-strand, α-helix.

Putative Siah Substrate Binding Sites.

The studies of Siah proteins until now strongly suggest that they function as E3 ubiquitin ligases by bringing together the the Ubcs and the proteins destined for ubiquitin-dependent degradation by the proteasome. The RING finger domains are known to bind Ubcs, while the C-terminal part of Siah is involved in the substrate recognition. Recently three structures of the complexes of ubiquitin ligases have been reported. One is c-Cbl protooncogene SH2 and RING domains in complex with the ubiquitin-conjugating enzyme UbcH7 and the peptide from its kinase substrate, Zap70 (Zheng et al., 2000). A second is of the complex of Skp1-Skp2, where Skp2 is a an F-box, LRR-domain protein, the substrate-recognition component of SCF ubiquitin-protein ligases (Schulman et al., 2000). A third is the VHL/elonginC/elonginB complex (Stebbins et al., 1999). None of these structures bear any similarity to Siah, and therefore they cannot be used to model or predict how Siah will recognise its substrates.

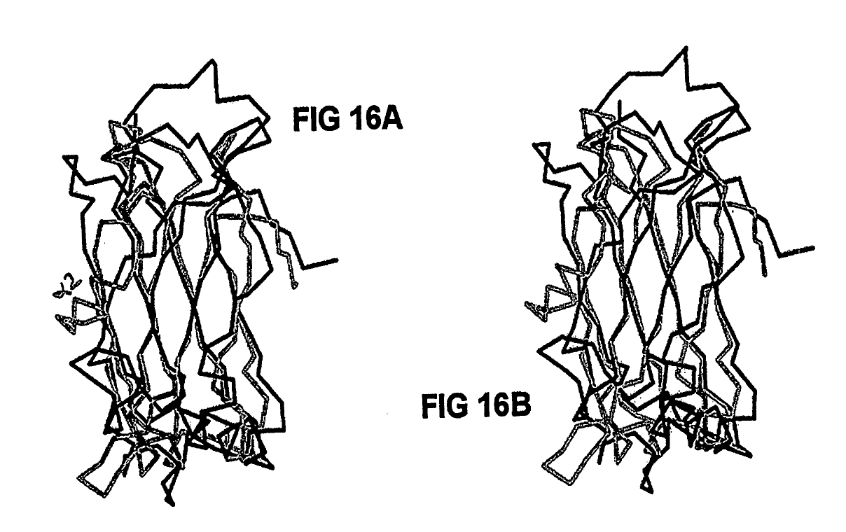

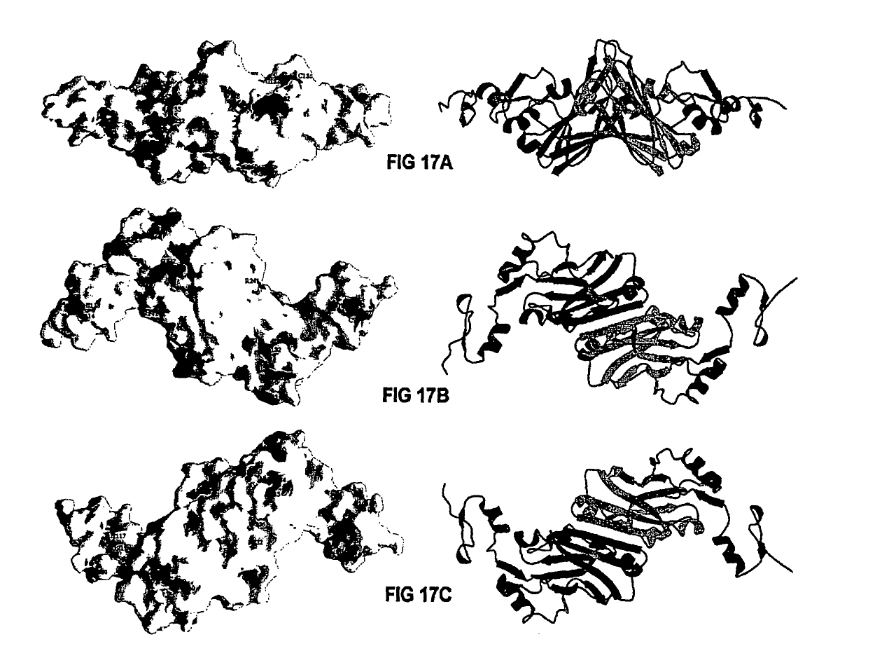

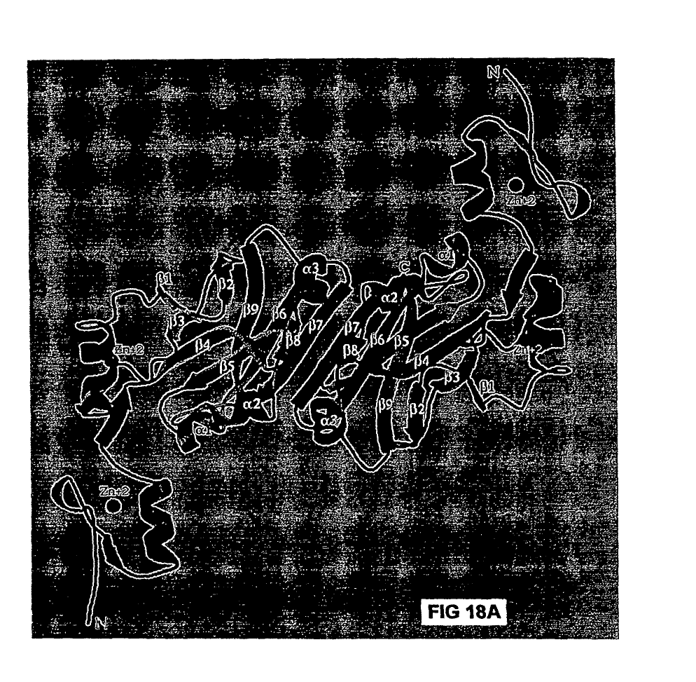

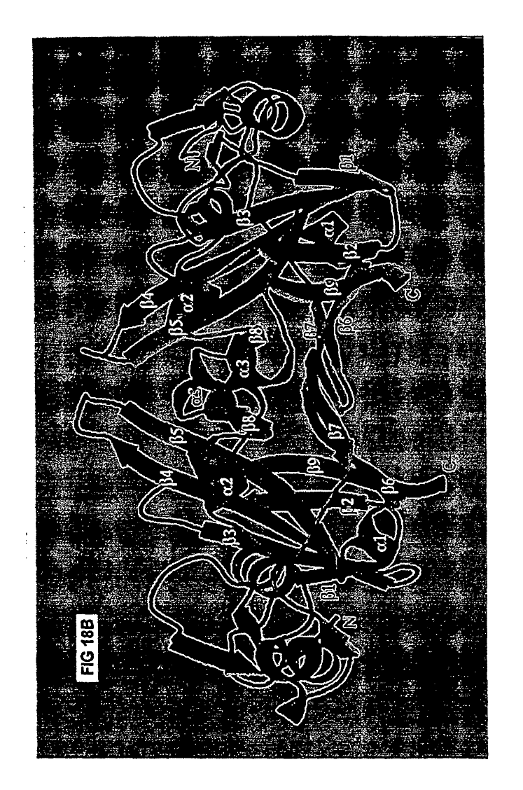

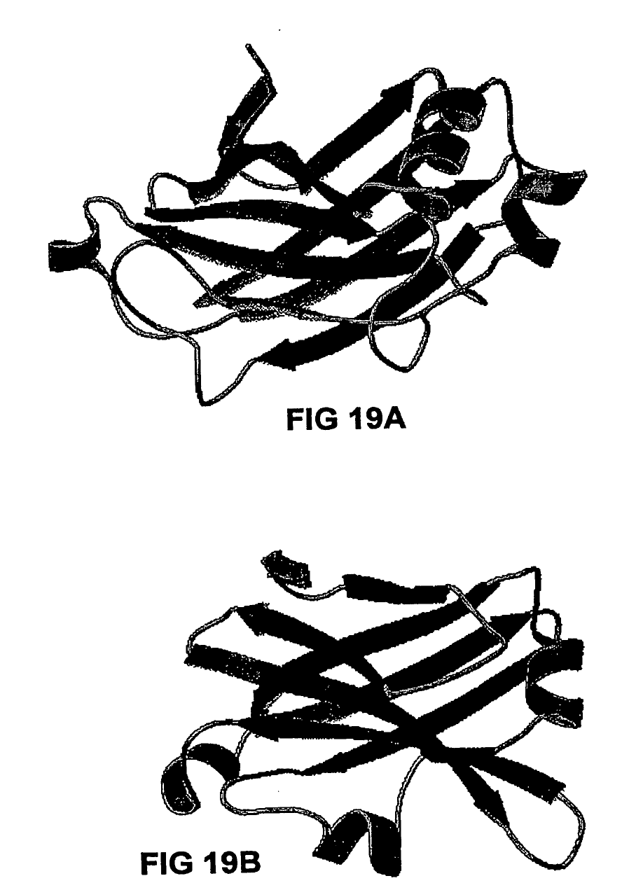

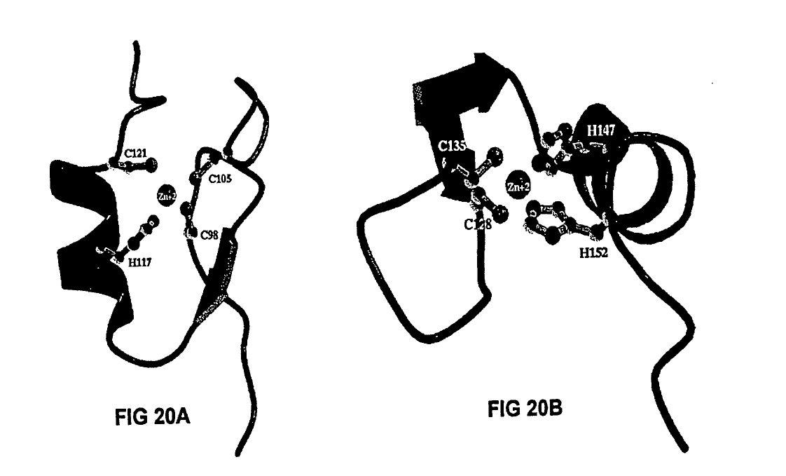

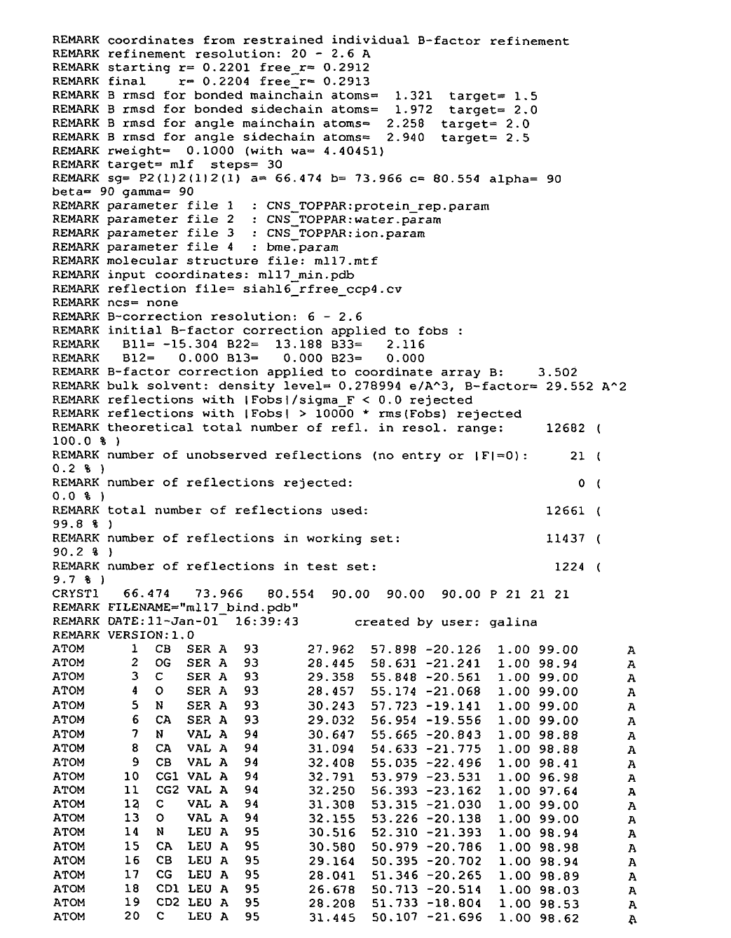

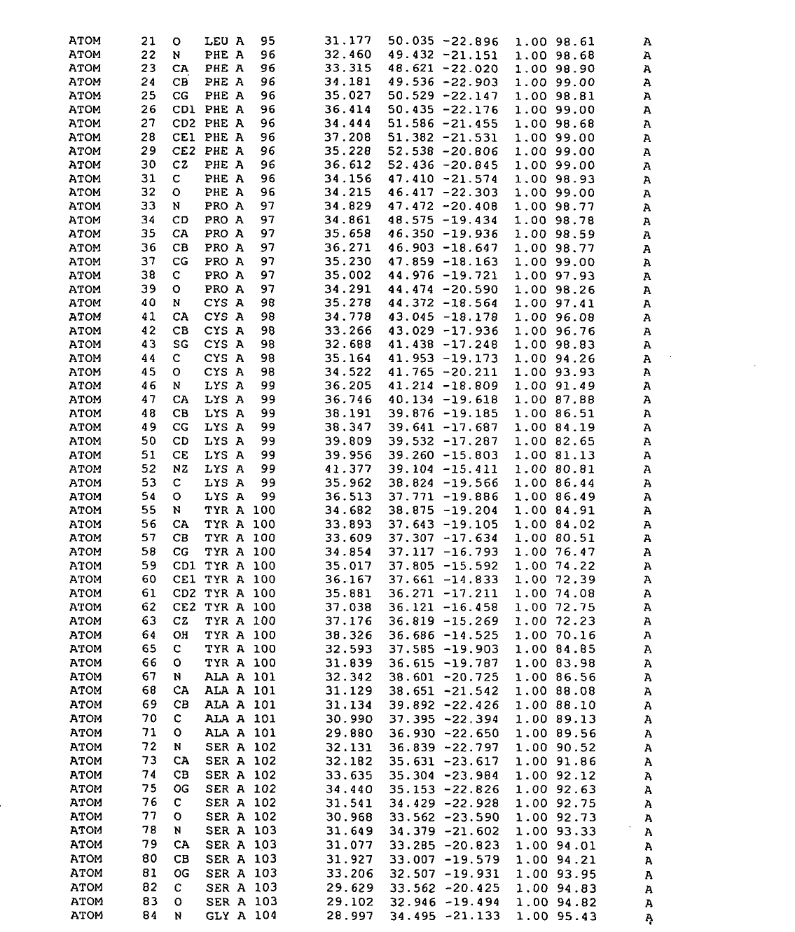

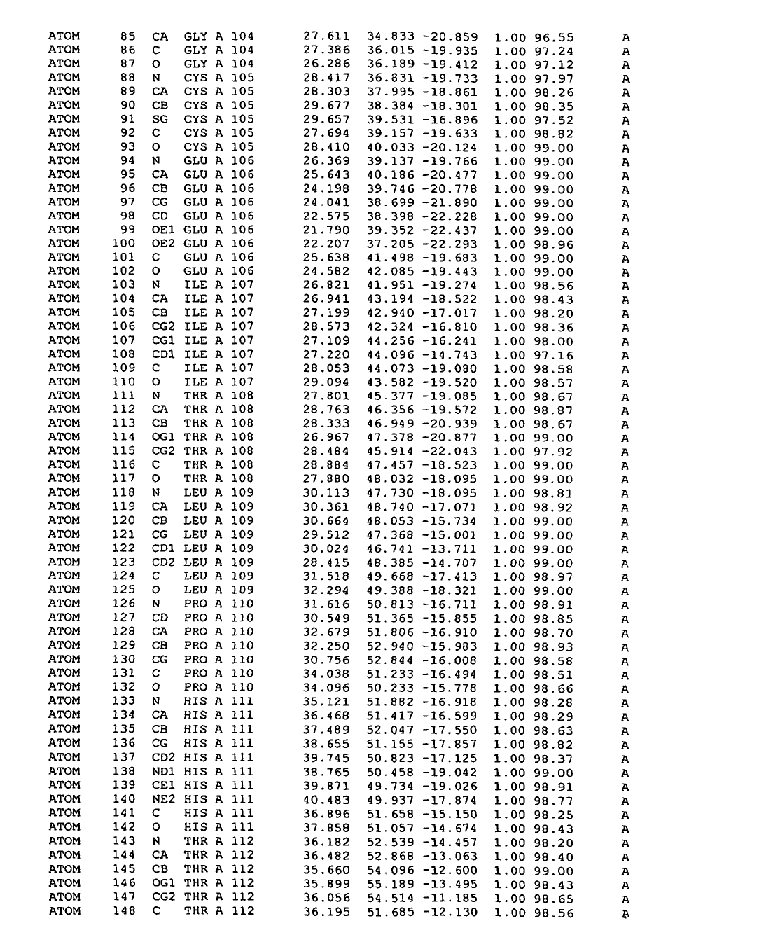

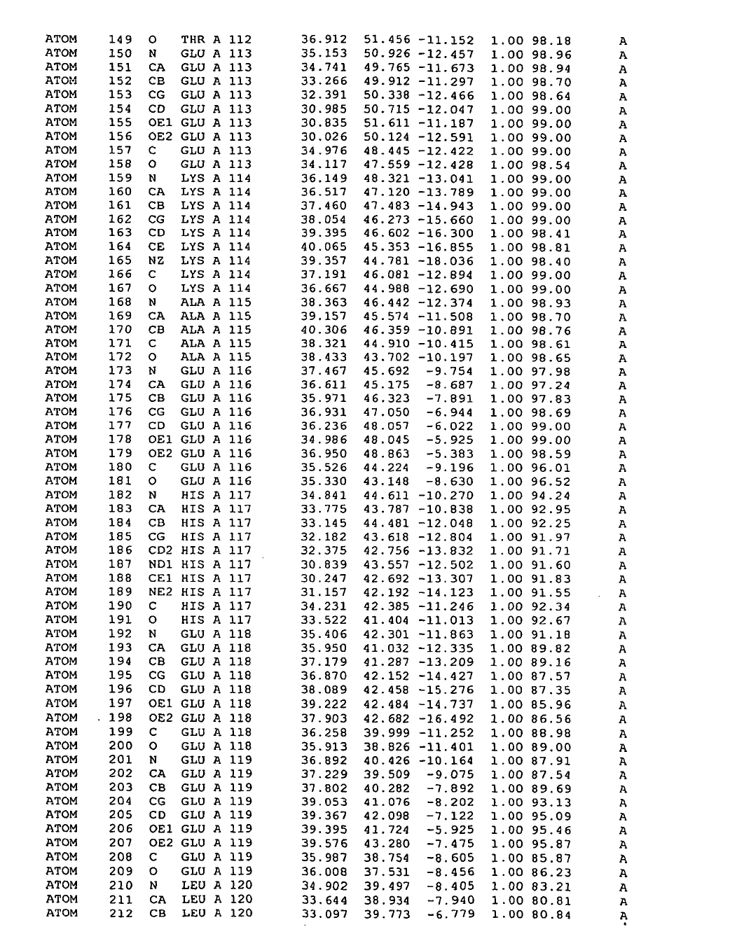

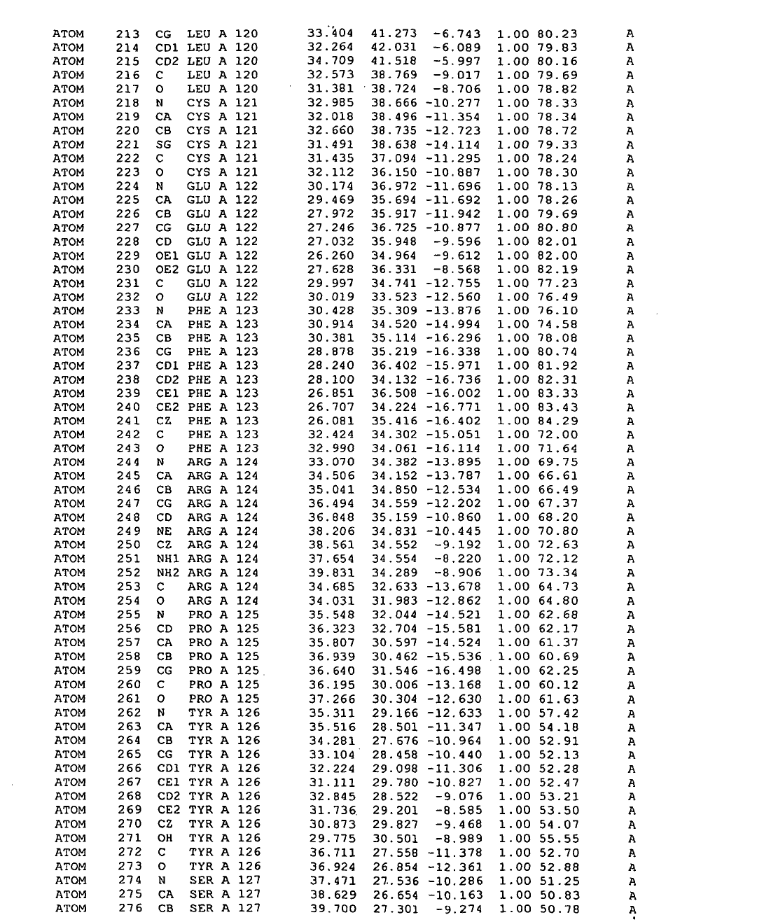

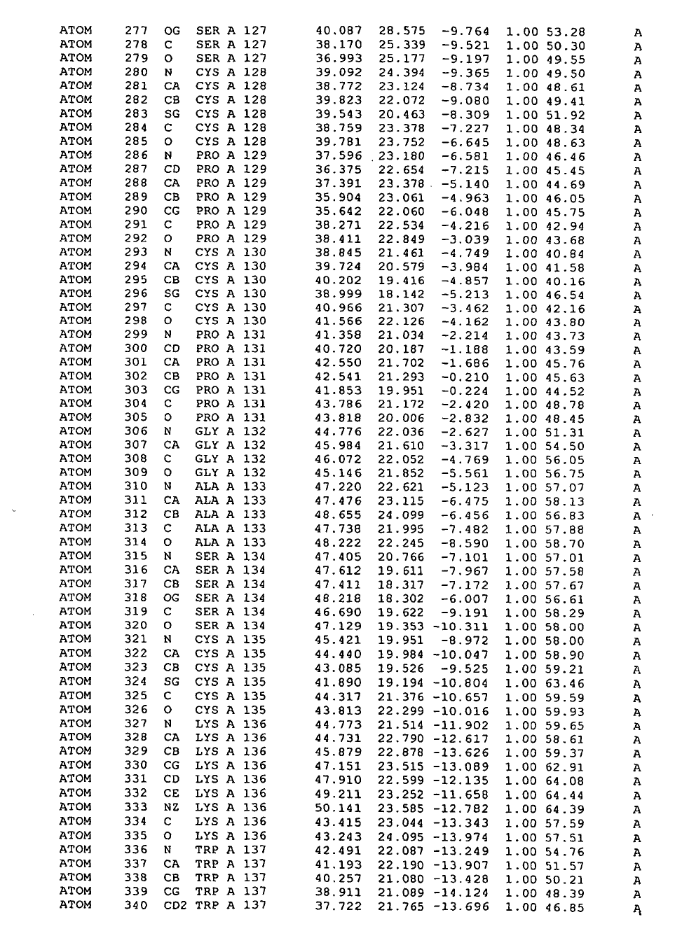

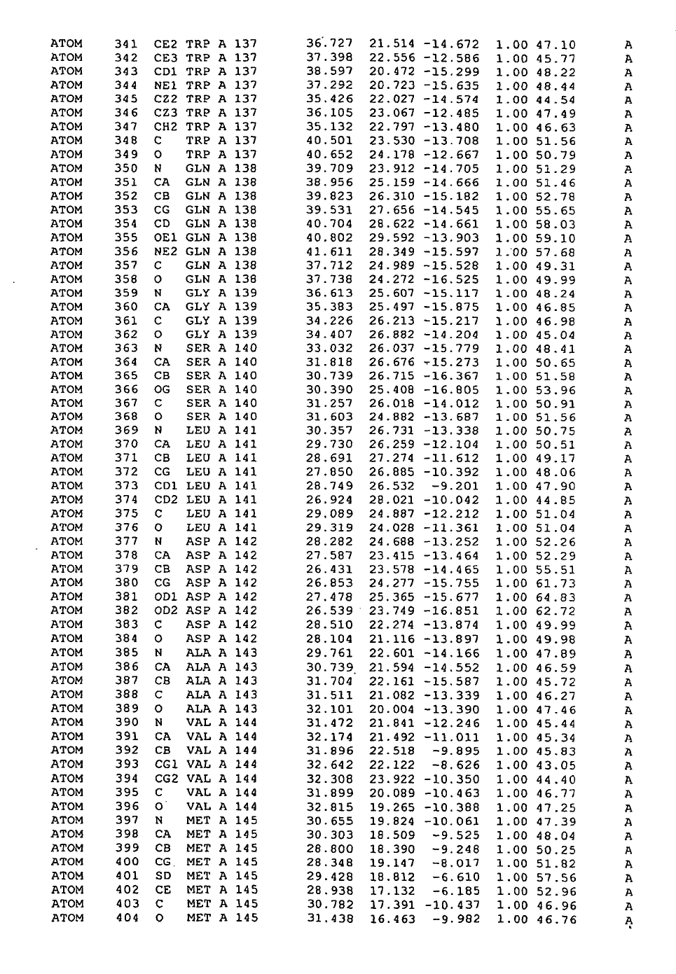

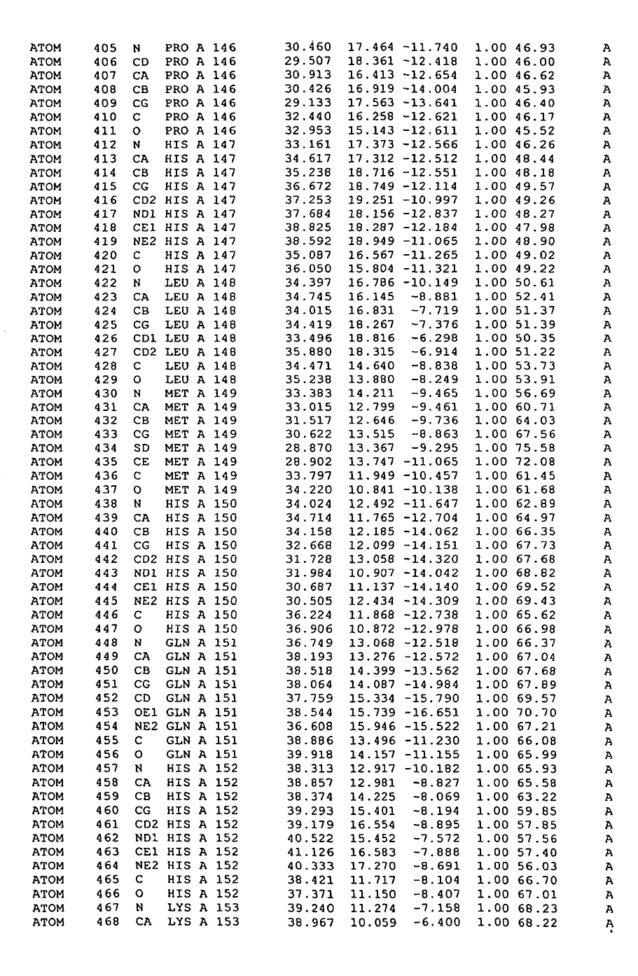

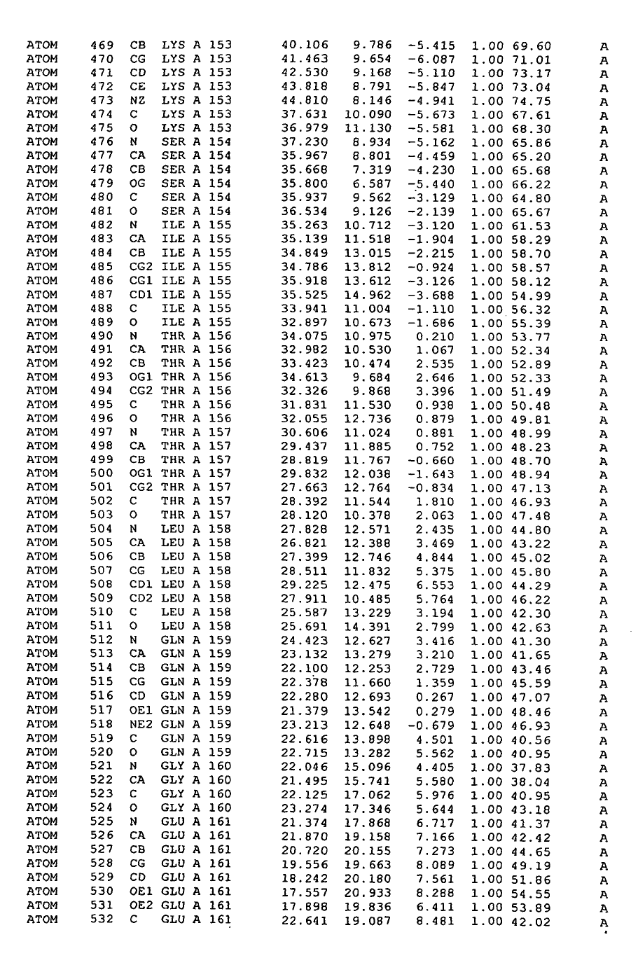

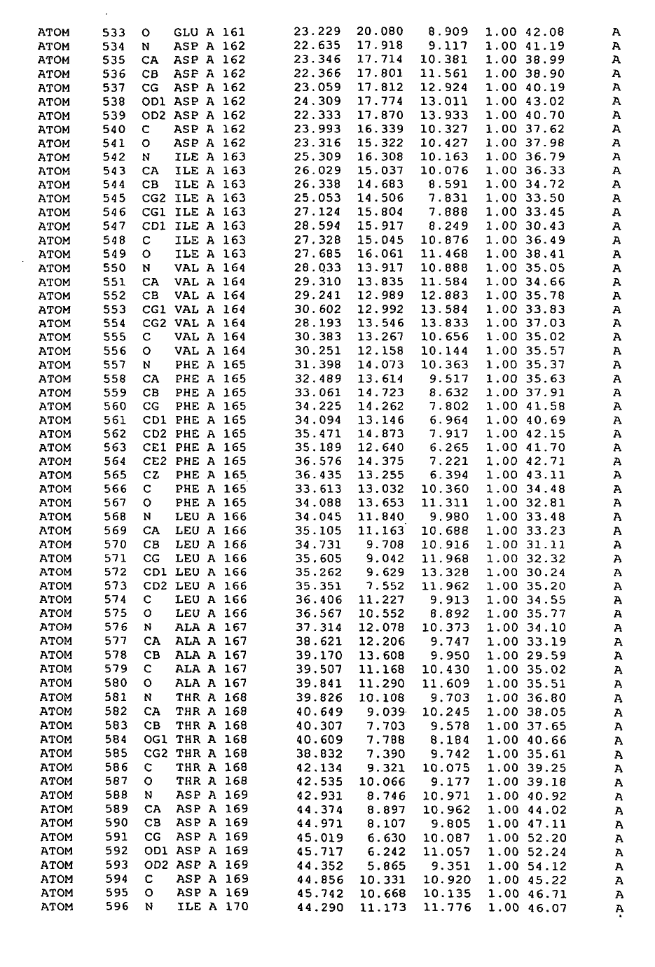

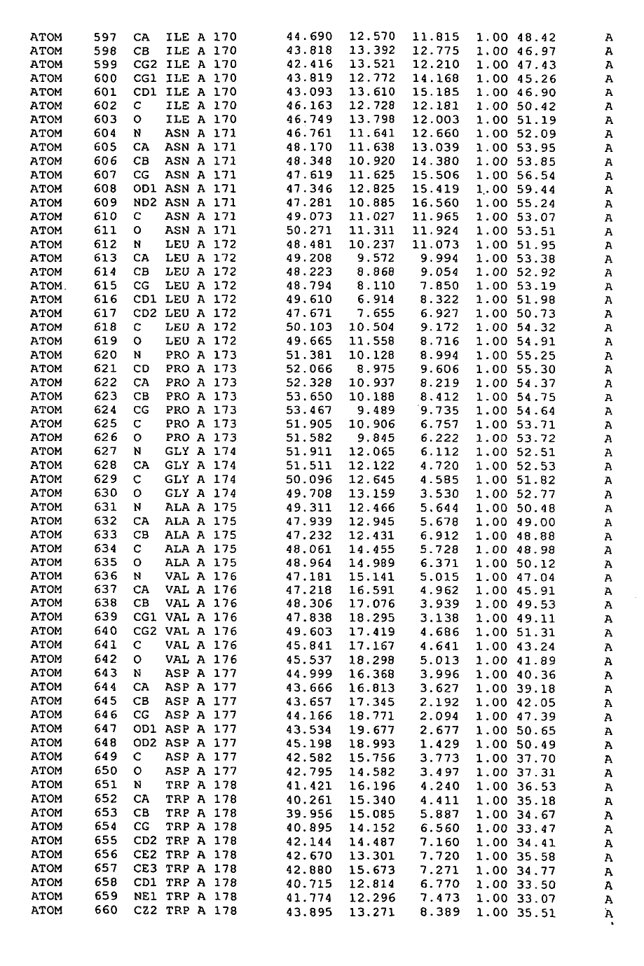

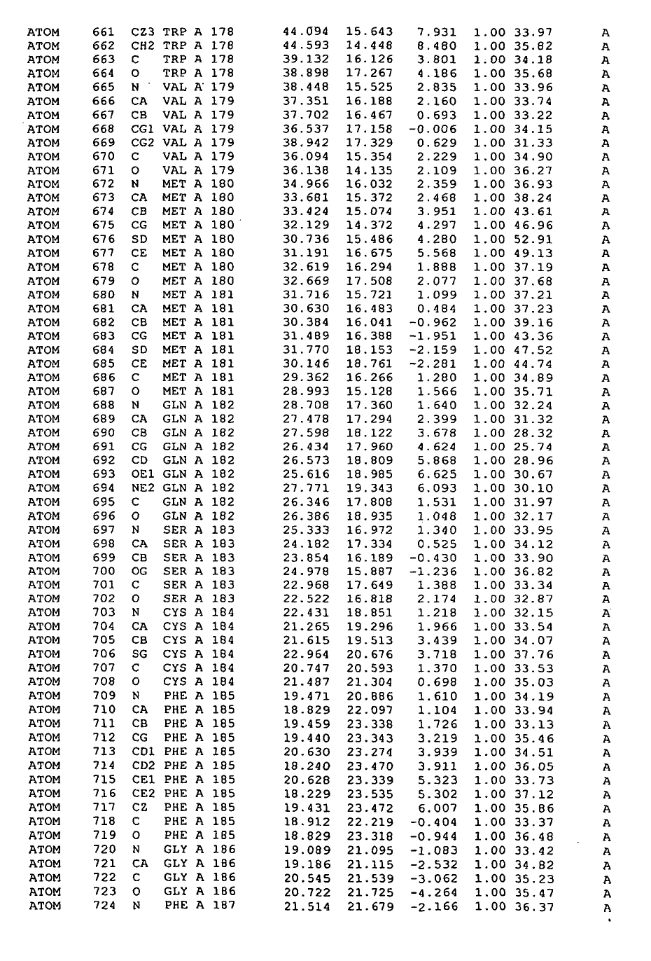

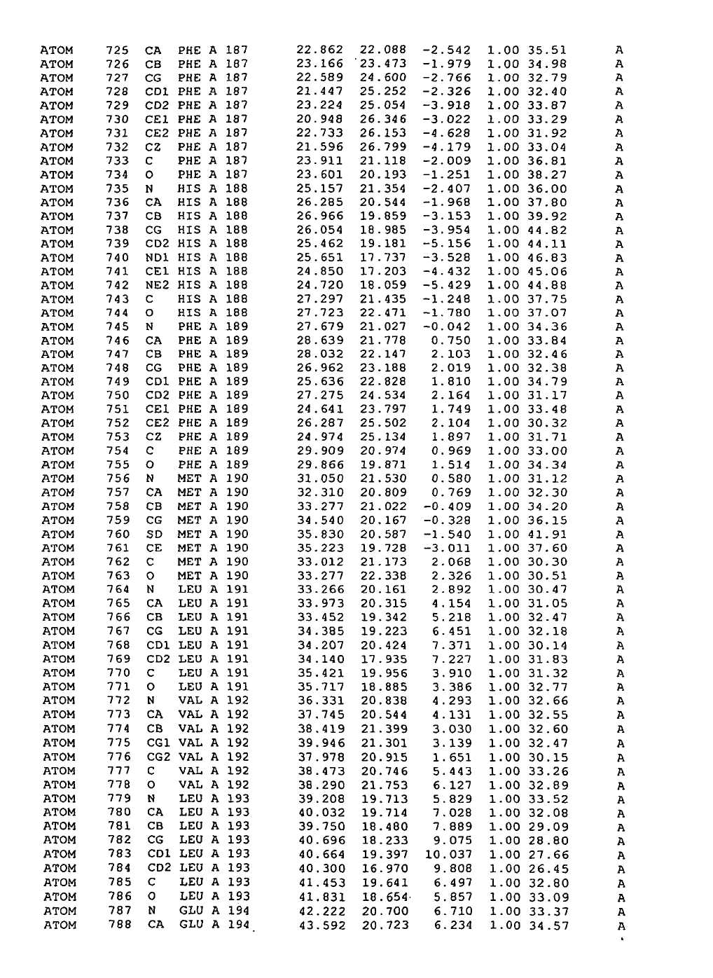

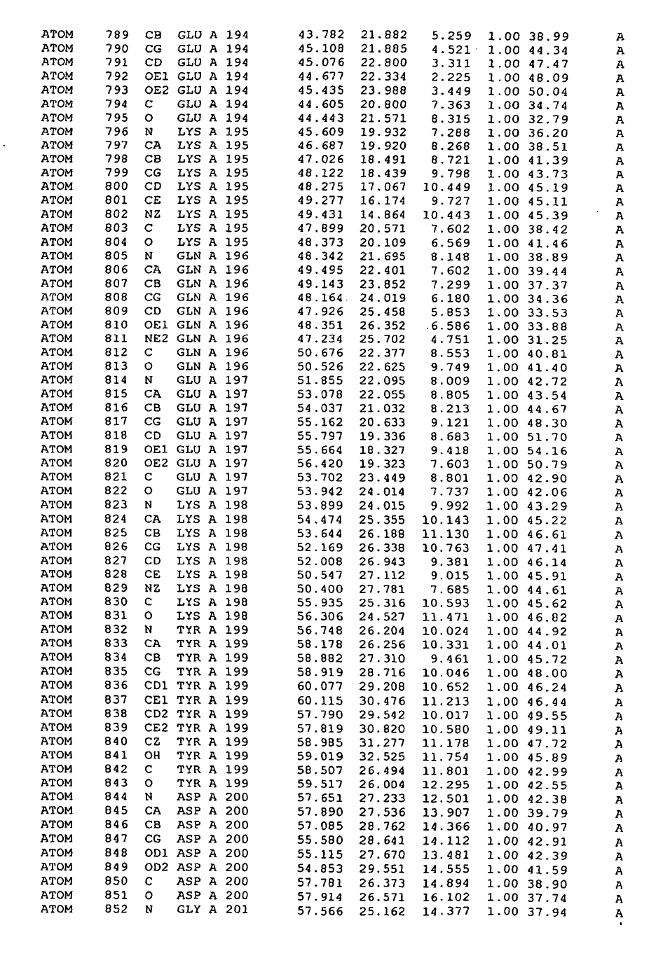

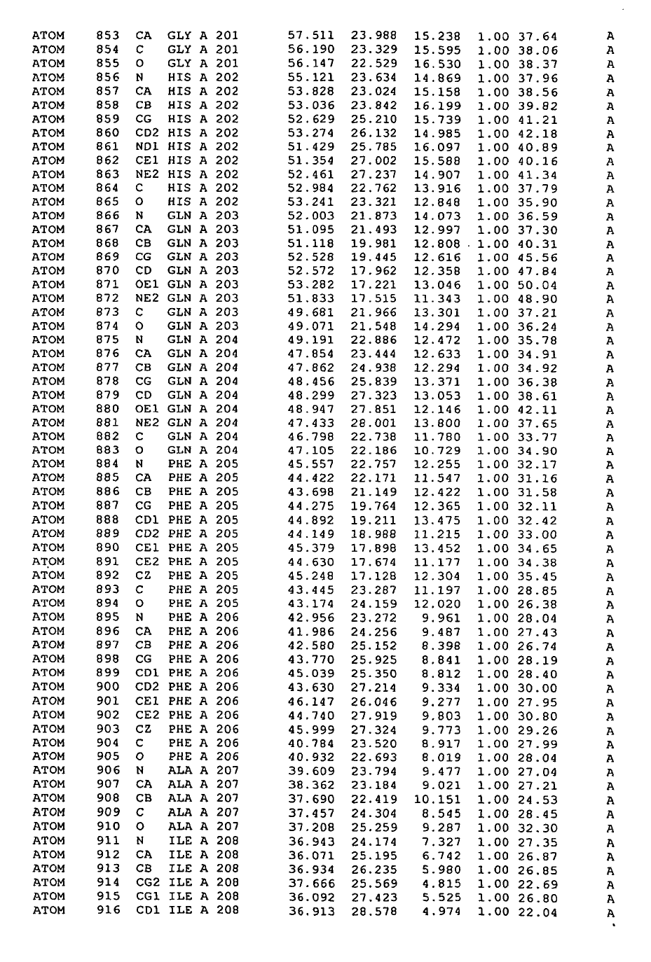

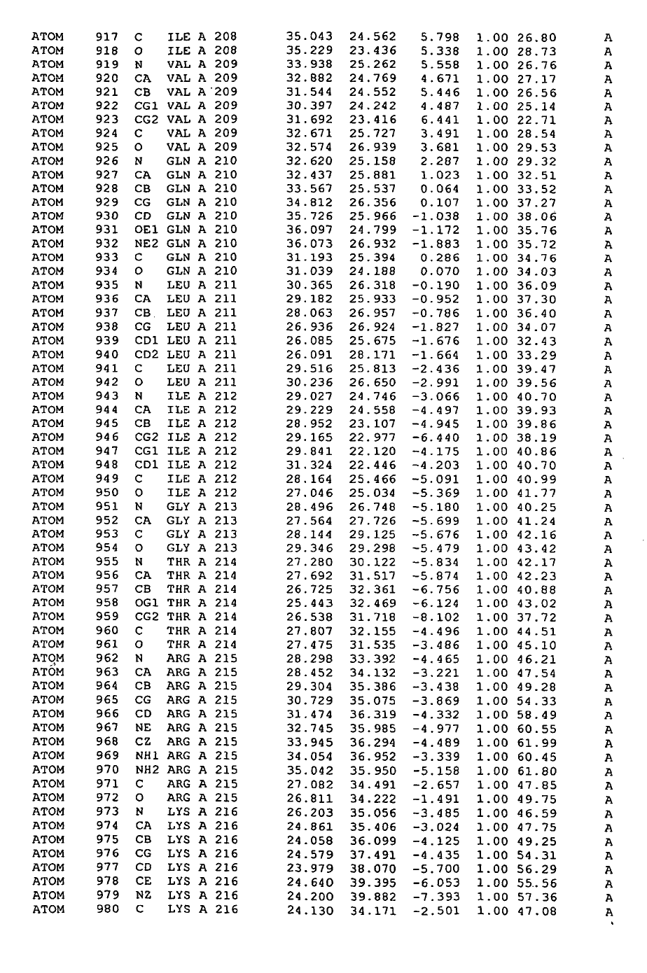

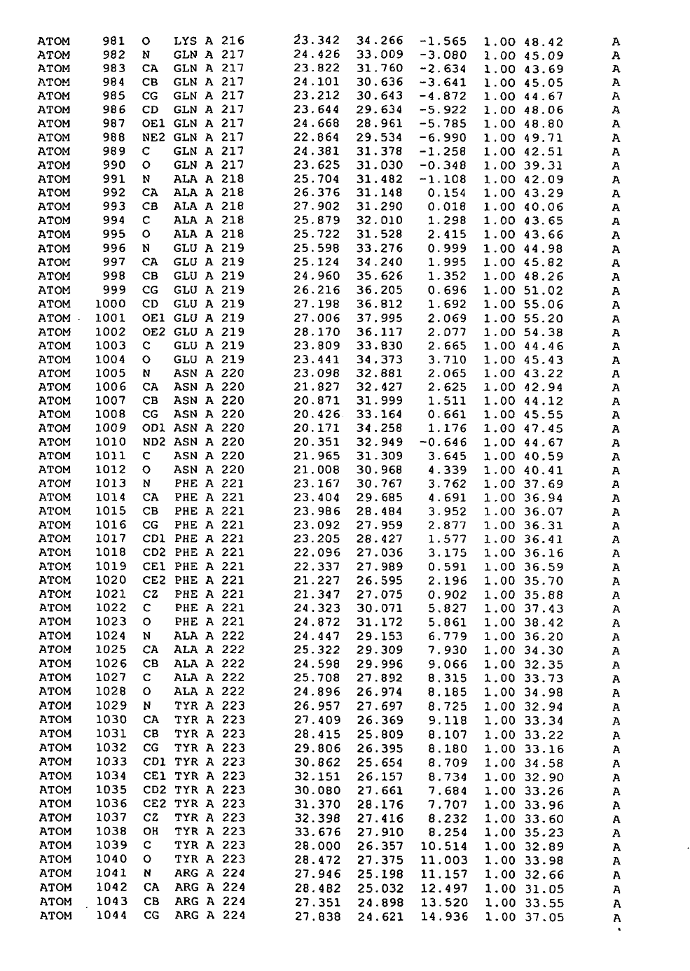

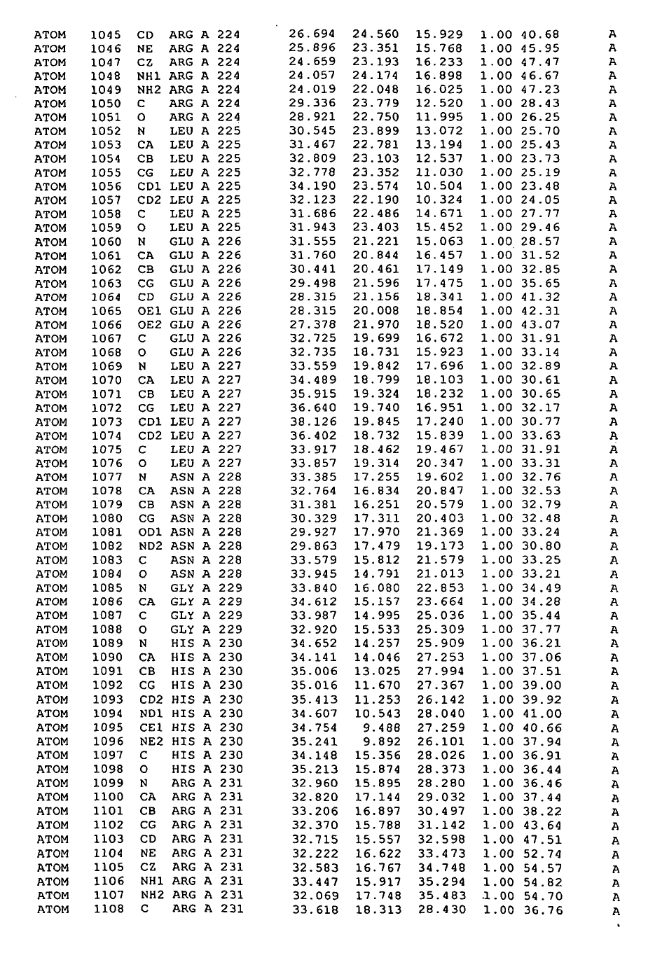

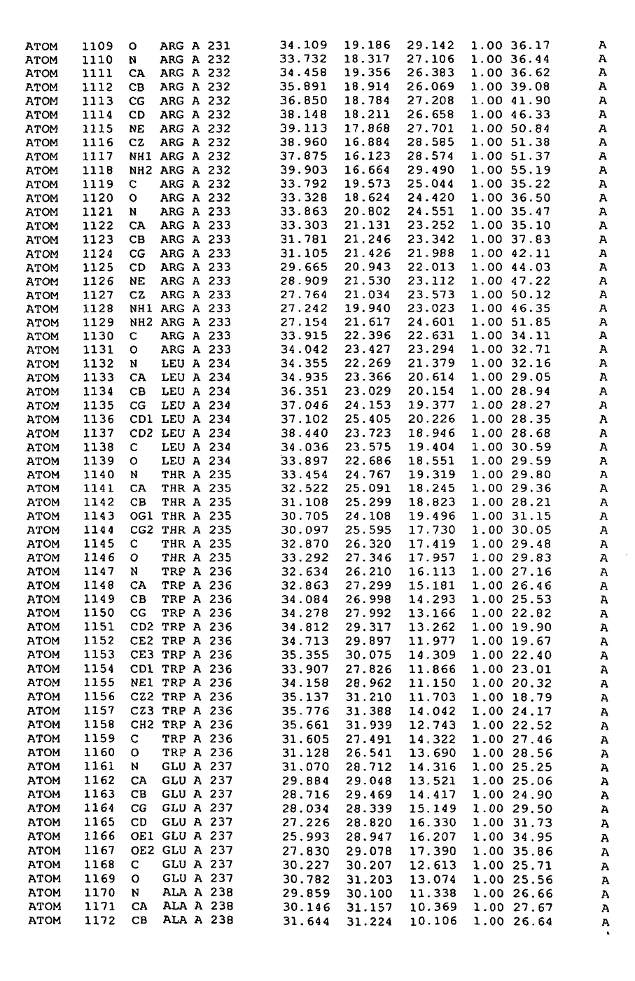

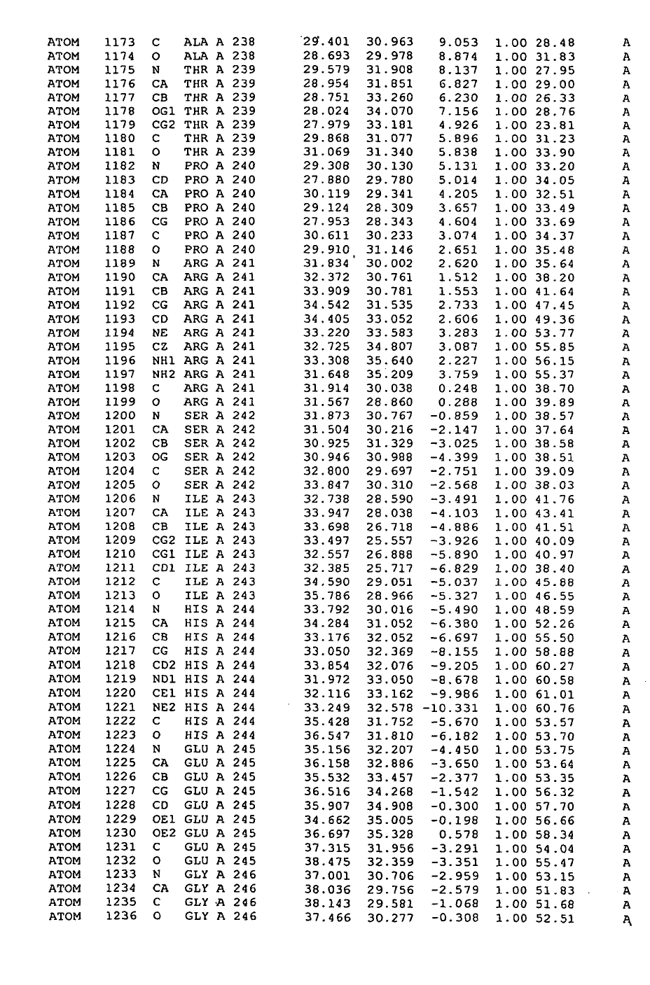

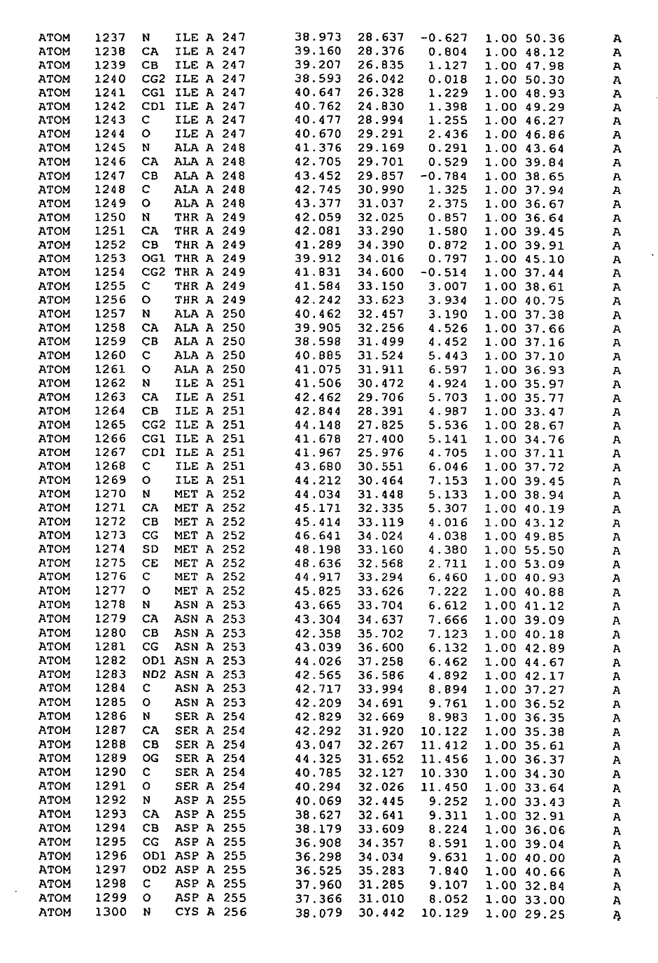

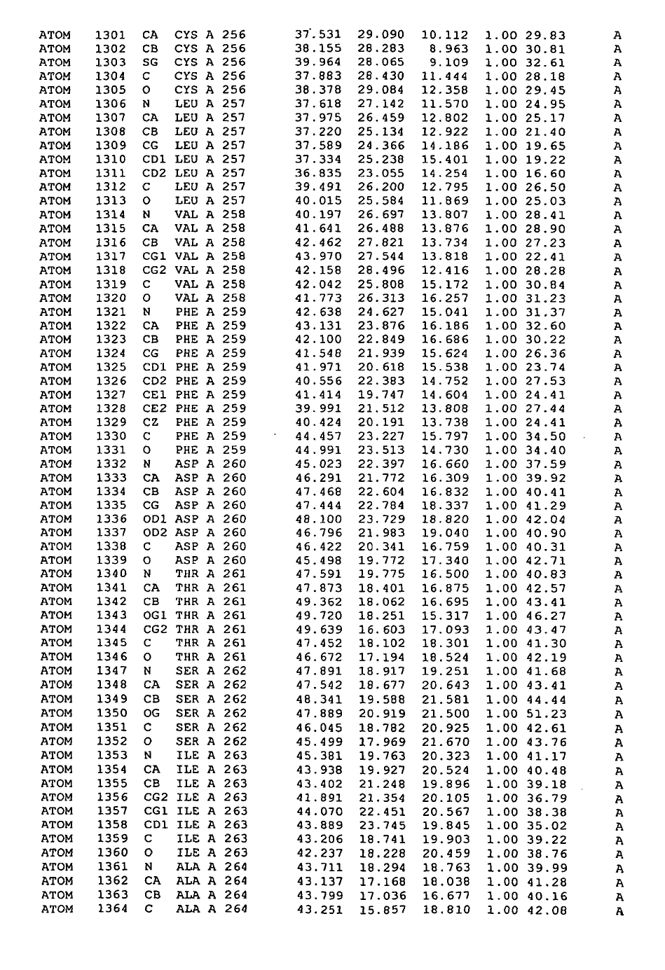

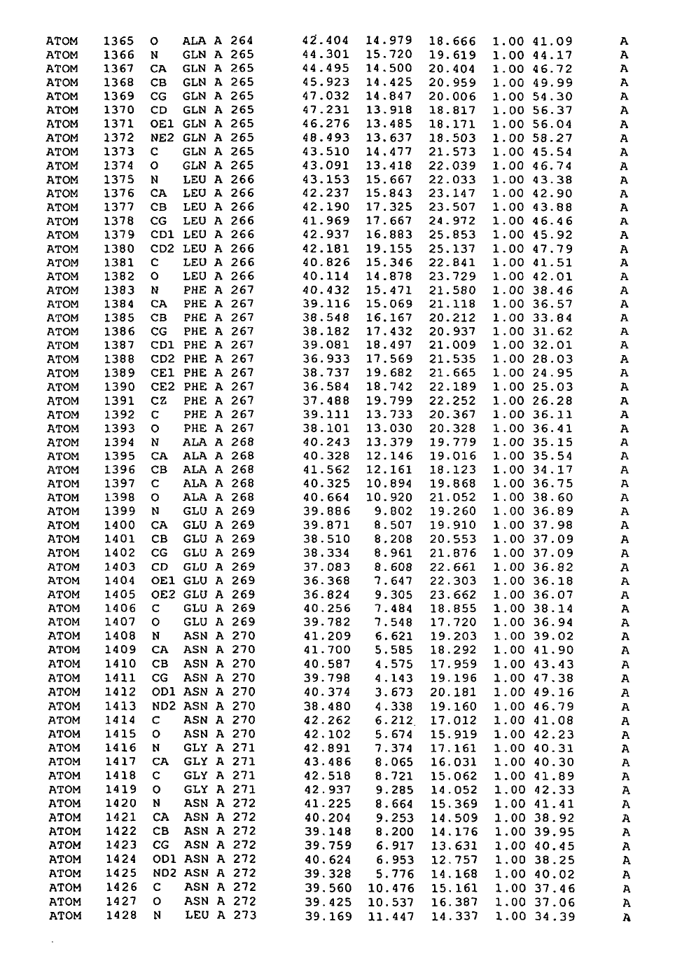

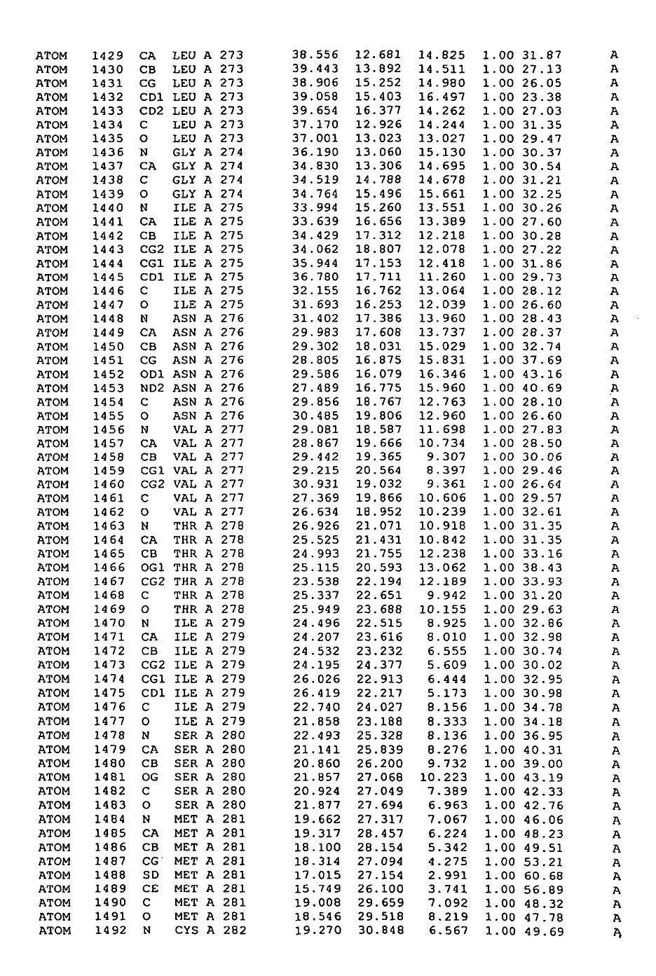

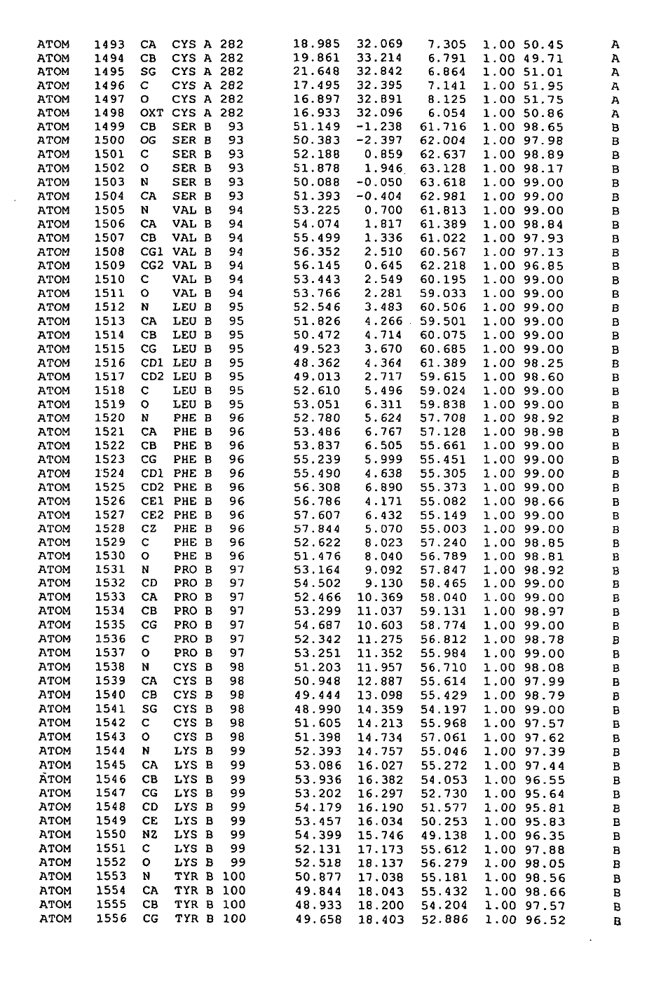

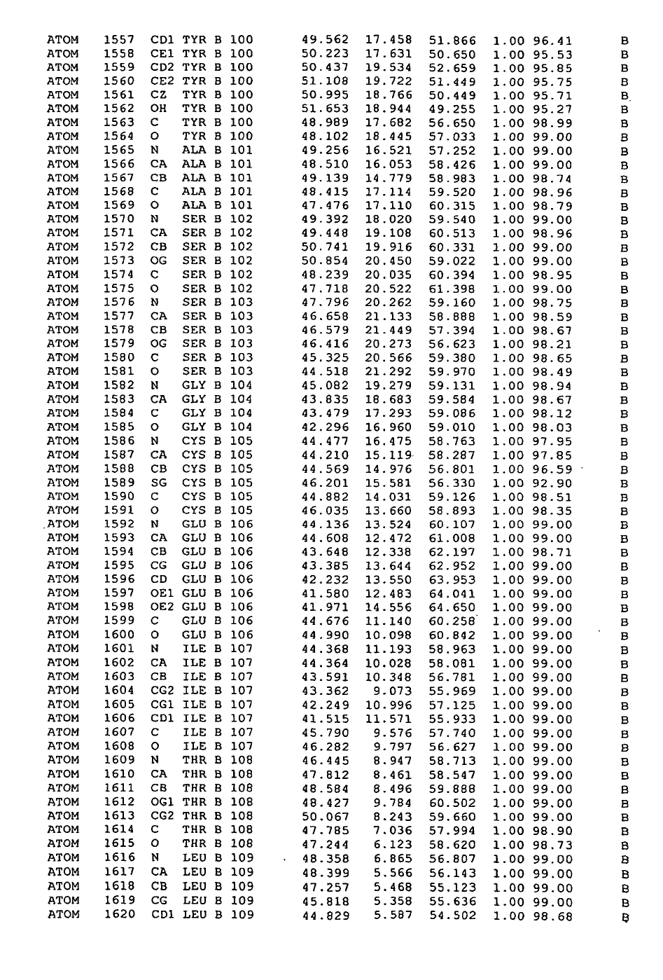

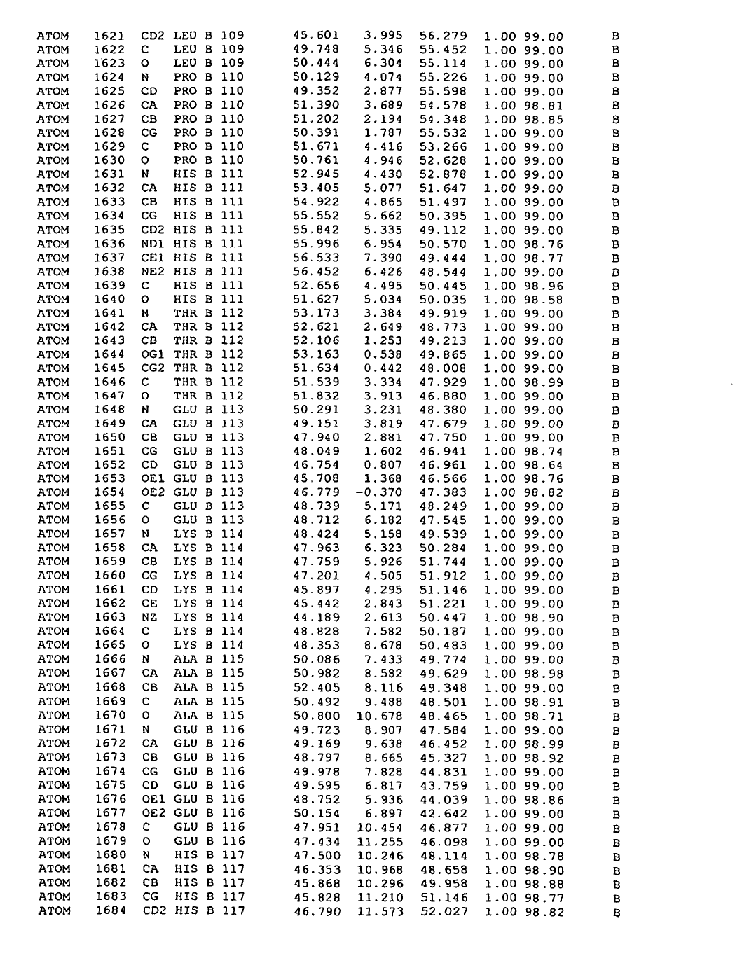

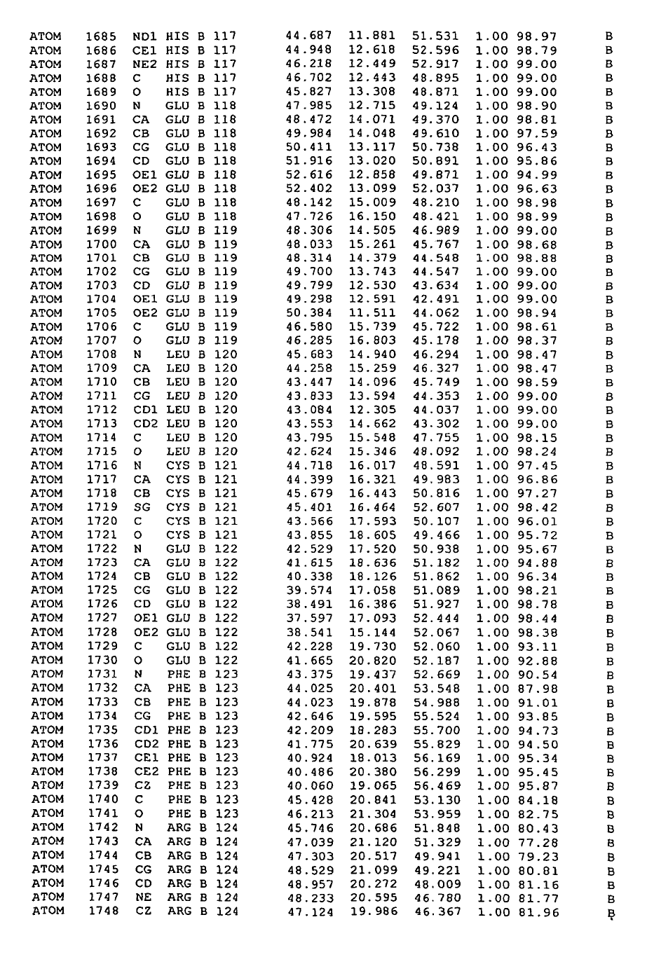

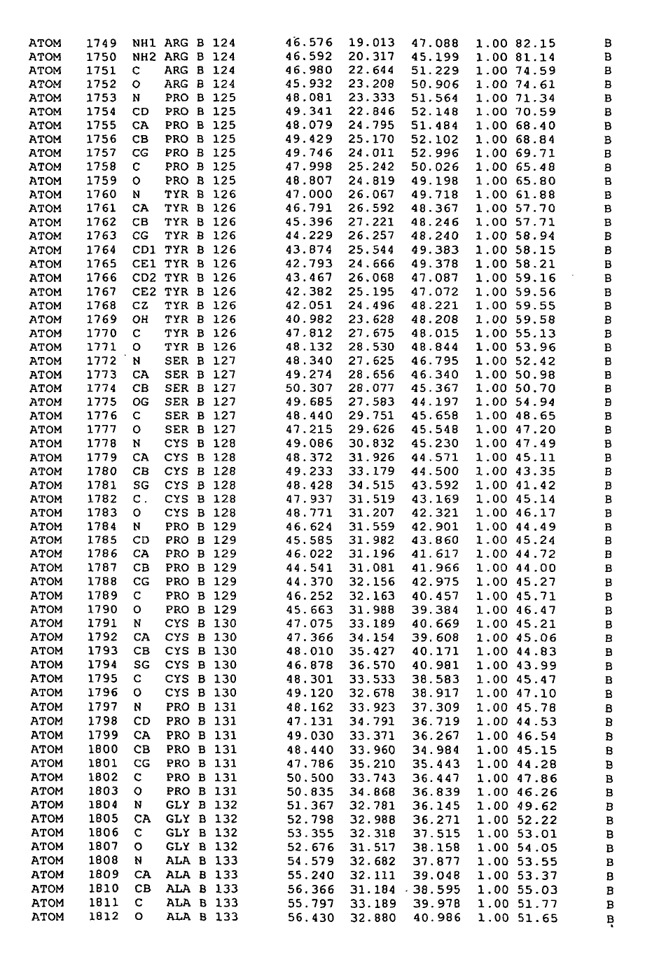

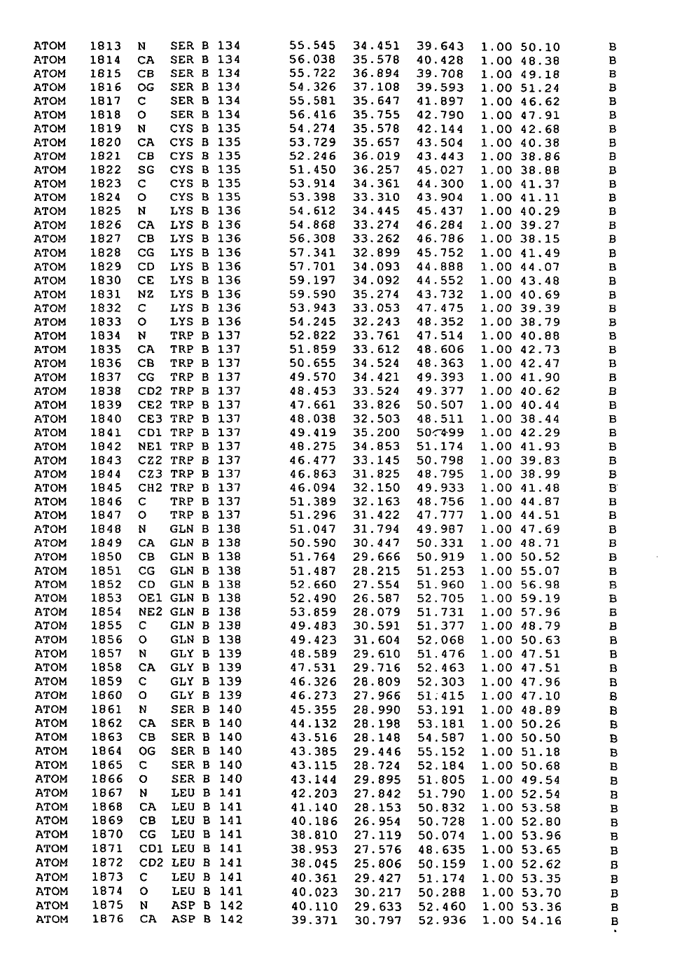

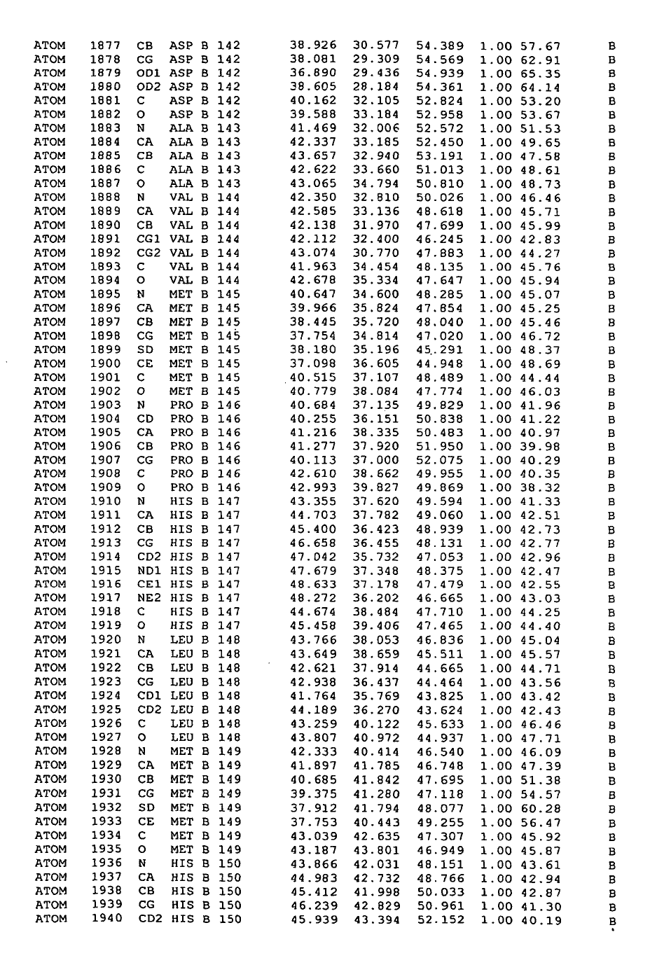

As mentioned earlier, the C-terminal domain of Siah has the same topology as TRAF. Indeed, 85 Cα atoms can be superimposed with the r.m.s.d. of 1.73 Å (FIG. 16). No sequence similarity exists between Siah and TRAF in their C-terminal domains. The structures of the C-terminal domain of TRAF2 in complex with the peptide from the TNF receptor-2 (Park et al., 1999) and in complex with the adaptor protein TRADD have been elucidated (Park et al., 2000). The binding mode for the peptide and TRADD differ markedly although they do occur on the same side of the β-sandwich structure. The peptide makes main chain hydrogen bonds with the part of the β-strand of TRAF that contains a β-bulge, as well as there is a hydrogen bond and a salt bridge formed between conserved residues of TRAF and the peptide. TRADD-TRAF2 interface is divided into two regions: one is largely hydrophobic and the other one is hydrophilic. Mutations of the hydrophobic residues drastically alter binding whilst mutations in the hydrophilic residues had minor effects (Park et al., 2000).

In Siah structure the location corresponding to the position of the peptide in the complex of TRAF with TNFR2 peptide is occupied by helix α2, and there is no β-bulges in any strands of Siah. A shallow groove is located on one side of this. The bottom of the groove consists of the strictly conserved residues among Siah and SINA proteins: Arg241 monomer A, Glu255 monomer A and Arg232 monomer B. Arg241 is found in the longest stretch of four strictly conserved residues in the C-terminal domain. The accessible surface area of the Arg241, Glu255, Arg232 patch is about 160 Å², which is the most prominent surface feature. The groove is circled by the residues 245, 249, 265, 230 of the C-terminal domain which are not conserved between Siah1 and Siah2. Siah1 and Siah2 are known to function differently in NFκB signalling, so residues within the binding site do not have to be conserved. The differences in Siah1 and Siah2 are also in positions 119,120 of the first zinc binding domain facing the Arg241, Glu255, Arg242 groove. There is also an exposed strictly conserved Lys114 pointing in the direction of the groove. These observations make this surface feature a possible binding site. The conserved Lys153 is in the vicinity and totally exposed. These data will however allow the design of inhibitors to any of the Siah family of proteins.

If TRAF2-TRADD complex is superimposed on Siah monomer there is no severe clashes in contrast to the TRAF2-TNFR2-peptide complex. However, the TRADD molecule occupies most of the space of the second monomer in Siah dimer, suggesting that this mode of binding is precluded whilst Siah is dimeric. Our observations suggest that the dimer is quite stable, a view reinforced by the large buried surface area in the dimer interface.

In short, prior structures of E3 complexes and the structurally related TRAF molecule do not elucidate the binding mechanism of the Siah binding domain.

Identification of Amino Acid Residues Relevant to Substrate Binding in Siah.

The Applicants have crystallised a Siah1a protein and have determined sites relevant to substrate binding. Without wishing to be limited by theory, it is believed that amino acid residues close to the surface of the binding domain are relevant to substrate binding.

In a preferred form of the invention the binding domain comprises amino acid residues found in the cysteine rich region (residues 98 to 152) and/or the C-terminal region (residues 152 to 282) of Siah1a or equivalent residues. Throughout the description and claims of this specification, the word “comprise” and variations of the word, such as “comprising” and “comprises”, is not intended to exclude other additives, components, integers or steps.

As used herein the term “equivalent residues” means residues that are in the same position in the Siah protein or have the same biophysical characteristics. It will be understood by the skilled person that two amino acid residues may not be in the same absolute position of a Siah protein sequence, yet still be equivalent. This is because all Siah proteins are not of the same length. However, it is possible to “align” Siah protein sequences of different origin based on amino acid sequence similarities along the length of the protein. This process would be very simple for the Siah family of proteins given the very high conservation of these proteins among species. After aligning the sequences of two Siah proteins it may be seen for example that amino acid at positions 123 and 124 align (and are therefore equivalent) because the two proteins differ in length by one amino acid.

The skilled person will also understand that certain amino acid residues have similar biophysical characteristics, and may therefore be substituted without having an appreciable effect on the biological activity of a protein. An example of a “conservative” amino acid substitution would be to substitute a nonpolar residue such as valine, for the residue isoleucine, that is of a similar size and hydrophobicity. There are many such substitutions available, and it is unnecessary to detail every possible substitution herein. The skilled person would only need utilise routine experimentation to find an equivalent residue for an amino acid in a given protein.

In a preferred form the binding domain comprises residues found in the region Leu 158 to Cys 282 of Siah1a or equivalent residues. More preferably the region comprises at least one residue selected from the group consisting of Arg 233, Thr 235, Glu 237, His 230, Asn 228, Glu 226, Arg 224, Ala 222, Asn 276, Thr 278, Ser 280, Met 281, Cys 282, Phe 185, Thr 168, Leu 166, Val 164, Asp 162, Glu 161, Gly 160, Leu 158, Arg 231 and Cys 184 of Siah1a or equivalent residues.

In another preferred form of the invention the domain comprises at least one residue from the region Cys 130 to Glu 194 of Siah1a or equivalent residues. More preferably the region comprises at least one residue selected from the group consisting of Trp 178, Lys 153, Thr 168, Thr 156, Phe 165, Val 179, Ser 154, Gly 174, Cys 130, Ser 134, Cys 135, His 152, Asp 177, Asp 169, Leu 172, Pro 173, Glu 194, Pro 131 and Leu 166 of Siah1a or equivalent residues.

In a further preferred form of the invention the domain comprises at least one residue from the region Glu 113 to Glu 269 of Siah1a or equivalent residues. More preferably the region comprises at least one residue selected from the group consisting of Arg 231, Arg 232, Glu 219, Lys 216, Arg 241, Glu 245, Arg 215, Asp 255, Asn 253, Leu 266, Glu 269, His 230, Gln 265, Met 252, Thr 249, Leu 120, Glu 119, Glu 116, Glu 113, Asn 270 and Ser 262 of Siah1a or equivalent residues.

In yet another preferred form of the invention the region comprises at least one residue from the region Glu196 to Ser262 of Siah1a or equivalent residues. More preferably the region comprises at least one residue selected from the group consisting of Asp 200, His 202, Tyr 199, Lys 198, Gln 204, Met 252, Ser 262, Asp 200, Gln 196, Asp 260, Gly 201, Thr 261 and Gln 203 of Siah1a or equivalent residues.

In still a further preferred form of the invention the domain comprises at least one residue from the region Pro 97 to Val 194 of Siah1a or equivalent residues. More preferably the region comprises at least one residue selected from the group consisting of Lys 114, Pro 97, Glu 118, His 111, Phe 96, Lys 99, Tyr 100, Arg 124, Glu 119, Ala 115 and Val 194 of Siah1a or equivalent residues.

In another preferred form of the invention the region comprises at least one residue from the region Cys 105 to Phe 123 of Siah1a or equivalent residues. More preferably the region comprises at least one residue from Cys 121, His 117, Cys 105, Ile 107, Leu 109, Phe 123, Leu 120 and Glu 122 of Siah1a or equivalent residues.

Dimerisation of Siah Proteins.

In a preferred form of the invention the binding domain according to is formed upon the dimerisation of two Siah proteins.

Preferably, the dimerisation occurs through the C-terminal domains of the Siah proteins. More preferably the dimer interface is formed by the residues Trp236, Phe267, Ile263, Leu234, and Val258 of Siah1a or equivalent residues.

In a preferred form of the invention the dimer interface comprises Glu219OE1-Arg231NH2, Arg232NE-Asp255OD1, Ser262OG-Ser254OG, Gln204, Asp260, Glu200monomer A, His202A, His202B and His150B-symmetry related.

Zinc Binding Domains

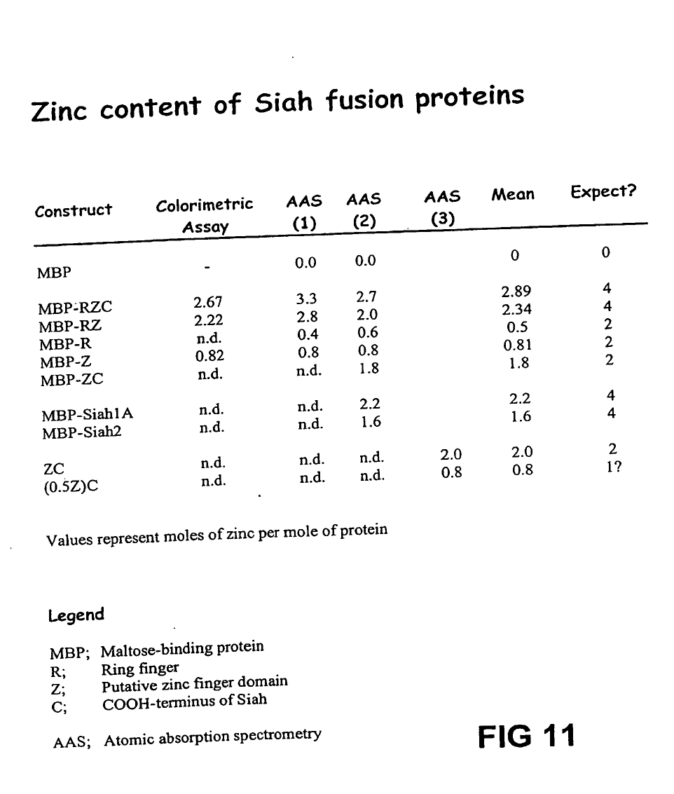

Another feature of the Siah protein is its ability to bind zinc. Sequence alignment of Siah family members across species from plants to humans highlighted the most conserved residues (FIG. 2). It was apparent from this alignment that the cysteine-rich region at the beginning of the C-terminal domain contained putative zinc-binding motifs. Zinc finger motif-based searches of the database had never predicted this region to be a zinc-binding domain because it does not fit the usual criteria assumed for zinc fingers. Part of this region had shown similarity to the cysteine-rich region of TRAF4 Matsuzawa, 1997, a region containing a number of putative zinc fingers fitting the motif C—X₂₋₃—C—X₁₁—H—X₂₋₃—C. The first and last of the zinc fingers in this stretch of seven diverge, with a C—X₆—C—X₁₁—H—X₃₋₄—C arrangement. The first of these bears sequence similarity to the cysteine-rich region of Siah. The possibility of that part of Siah binding zinc has not been reported until the present invention. Initially, zinc measurements were made on MBP-Siah fusion proteins to confirm that the cysteine-rich region did in fact bind zinc. These measurements were done initially using a colorimetric assay which was not highly specific for zinc (FIG. 11), but were followed with atomic absorption spectrometry (FIG. 11), confirming that the C-terminal region did bind zinc in addition to that coordinated in the RING domain. Subsequent measurement of zinc in the purified binding domain gave a stoichiometry of 2.0 mole of zinc per mole of protein. A shortened construct, lacking the first four putative zinc-coordinating residues, bound 0.83 mole of zinc per mole of protein, suggesting that this domain consisted of two separate zinc-binding regions, rather than a single structural domain (FIG. 11). These results were useful for solving the crystal structure, as the naturally coordinated zinc was used to solve the phases of the X-ray diffraction data, a critical step in the crystallographic process.

The N-terminal part of Siah molecule is rather mobile, as manifested by high B-factors and weak electron density, especially in the first zinc binding domain. The second zinc finger domain has an overall structure similar to the classical zinc fingers of TTK (Schwabe and Klug, 1994), with a ββα arrangement. This particular type of zinc finger is generally found in transcription factors and is responsible for DNA binding. However, more recent data has emerged showing that many zinc fingers with this topology can mediate specific protein-protein interactions (Liew et al., 2000). Despite the overall similarity, there are number of subtle differences between the Siah zinc finger and the classical transcription factor zinc finger. The loop between two first Cys ligands is much longer, 6 residues rather than 2-4, and the spacing between the last two His ligands is longer by a residue. Zinc is coordinated by Cys128, Cys135, His147, His152, as predicted from sequence alignments. There is a cysteine residue Cys130 in the long loop connecting Cys128 and Cys135 and a molecule of P-mercaptoethanol bound to Cys130. His150 in monomer B is the ligand for the zinc found in the dimer interface. The zinc ion of the first zinc finger domain is coordinated by Cys98, Cys105, His117 and Cys121.

There is an extra zinc ion coordinated by His230 and Glu269 of monomer A and by the corresponding residues from monomer B. This zinc binding site may be physiologically irrelevant, because the zinc ligands are provided by two symmetry related molecules in the crystal, and such interaction is very unlike to exist in the solution as Applicants have shown by extensive gel filtration experiments. In addition, His230 and Glu269 are not conserved among Siah and SINA proteins (FIG. 2).

Similarly, zinc in the dimer interface involves the residues that are not strictly conserved among the Siah and SINA families, however, the His 202Tyr mutation in fly has a mutant phenotype, suggesting that this residue has some structural and functional relevance.

The zinc finger domains of each monomer are most distant from the non-crystallographic 2-fold axis and are not involved in the dimer formation. 1128 Å² of each monomer surface is buried upon dimerisation, indicating a very tight association of two monomers. The center of the dimer interface is formed by the hydrophobic residues: the conserved Trp236 and Phe267, as well as Ile263 Leu234, Val258. The latter three residues are not strictly conserved among Siah and SINA sequences (FIG. 2), but are conservatively substituted by other hydrophobic residues. This hydrophobic core is stabilized by a network of the main chain hydrogen bonds formed by an anti-parallel beta sheet between β7 strands from each monomer. These interactions cause an extended parallel eight β strand sheet to be formed across the dimer interface. In addition, there are six side chain-side chain hydrogen bonds between the monomers that contribute to the dimer interface. These are Glu219OE1-Arg231NH2, Arg232NE-Asp255OD1, Ser262OG-Ser254OG. There is also a water-mediated contact between Gln204 and Asp260. Finally, the zinc ion coordinated by Glu200monomer A, His 202A, His 202B and His150B-symmetry related seals the dimer interface at the tip of Siah molecule. Noticeably, the conformations of the loop β4-β5 in two monomers, where the zinc ion is bound, are different, and therefore do not obey the two-fold non-crystallographic symmetry.

Potential Recognition Motif for Siah Binding Domain

Interaction studies using bacterially expressed proteins have yielded only one high affinity interactor, the phyllopod (PHYL) protein from Drosophila, with other proteins interacting with lower affinity (deleted in colorectal cancer (DCC), kinesin-like DNA-binding protein (Kid), Siah-interacting protein (SIP) and Oct-binding factor 1 (OBF-1)). PHYL is known to interact and function with SINA, the Drosophila orthologue of mammalian Siah, in the targeted degradation of the transcriptional repressor tramtrack 88 (TTK88). Applicants have shown that it interacts similarly with mammalian Siah.

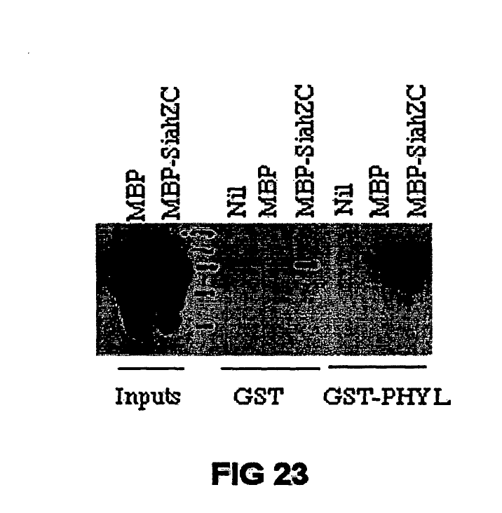

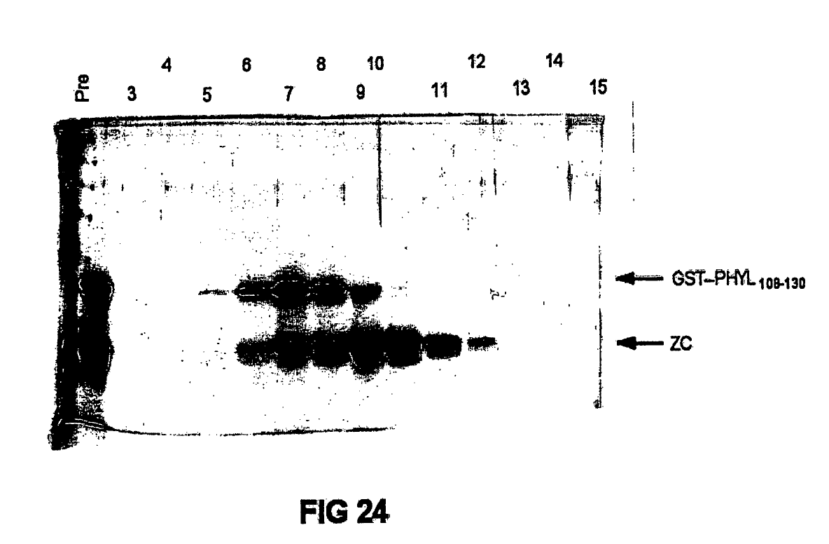

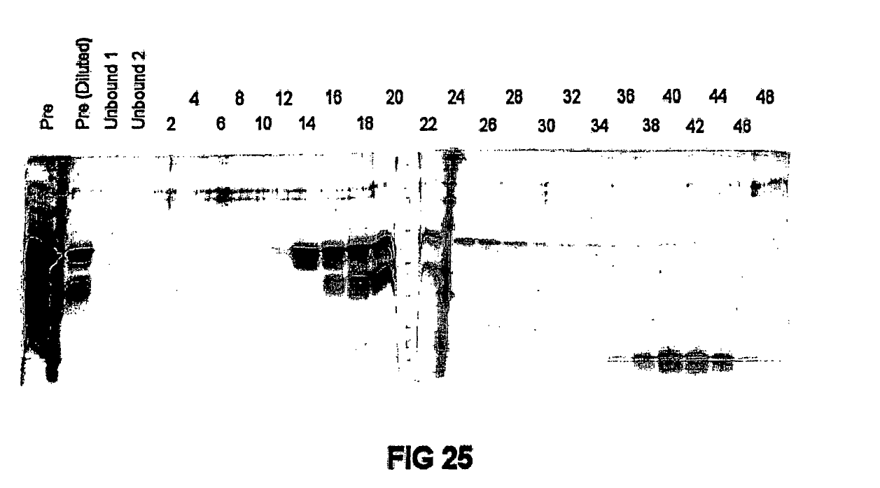

A previous report (Kauffmann et al.) suggested that SINA may bind to a minimal fragment of PHYL (namely residues 108-130). We have tested Siah and SINA binding to this peptide and shown that it is sufficient to give high affinity binding. We initially did this by expressing the peptide as a fusion protein with glutathione-S-transferase (GST) and using glutathione-Sepharose beads to localise the fusion protein to the solid phase. Binding of Siah and SINA was measured in a pull-down assay (FIG. 23). Initial attempts at purifying a complex suggest that the complex is stable under conditions employed for gel filtration on Superdex 200 (FIG. 24) and anion exchange chromatography on a Mono Q column (FIG. 25).

The stoichiometry of interaction was investigated by isolating a complex of GST-PHYL108-130 and Siah1a SBD by gel filtration on Superdex 200 and quantitating the components by N-terminal sequencing. After five cycles of sequencing the average ratio of Siah binding domain to GST-PHYL108-130 residues was 2.34±0.38, suggesting that one PHYL peptide bound per Siah dimer.

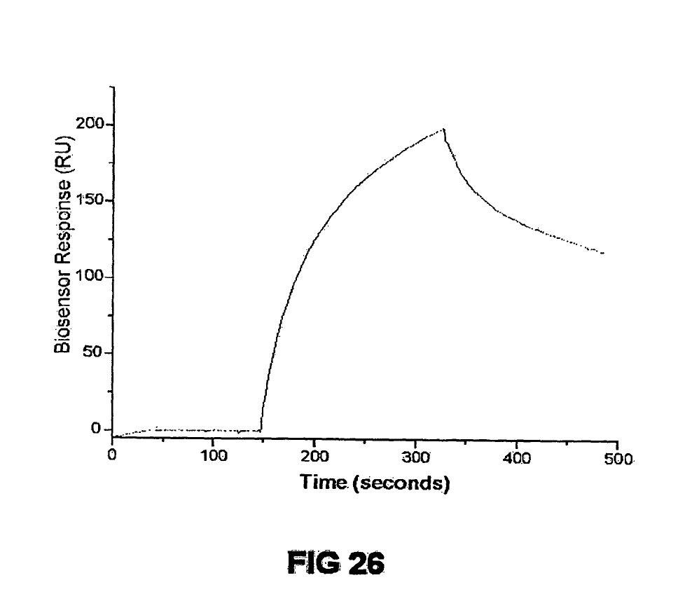

To determine binding affinities, synthetic PHYL peptide was immobilised to a C5 BIAcore chip using two different strategies (direct chemical coupling through the free amino group on Lysine113 and binding to coupled avidin via an N-terminal biotin on the peptide) and interaction was measured directly by surface plasmon resonance. This method facilitated the measurement of association and dissociation rates, and the determination of an affinity constant (FIG. 26). The affinity constant is approximately 180 nM.

From the work described above it appeared that the PHYL peptide contained most or all of the requirements for high affinity binding to the Siah binding domain. Computer alignment of this sequence (using ClustalW, Angis) with the published sequences of interaction domains/fragments from other proteins, suggested that there may in fact be a binding motif (PXAXVXP) in the middle of the peptide (FIG. 2).

In order to test this hypothesis Applicants systematically mutated the PHYL peptide, utilising the solid phase pin technology of Mimotopes (Clayton, Victoria, Australia). A full set of peptides, with alanine substituted at each position in the peptide, was synthesised. At positions where a native alanine occurred, glycine was substituted. Mutant peptides were screened for their ability to compete with the binding between Siah and PHYL peptide, then peptides of interest were investigated further utilising surface plasmon resonance.

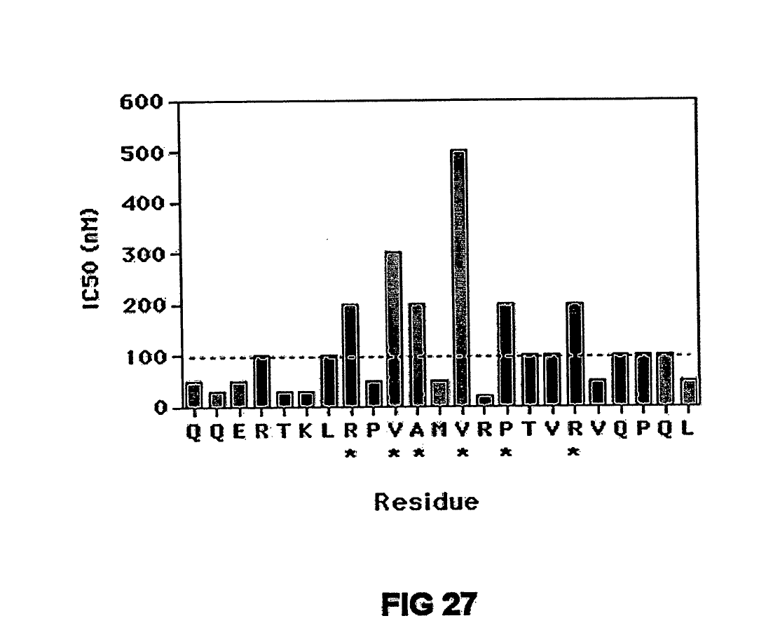

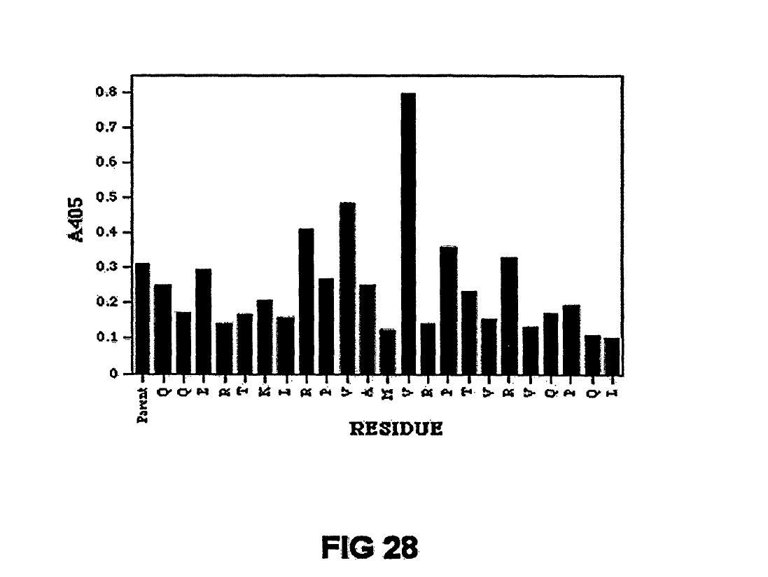

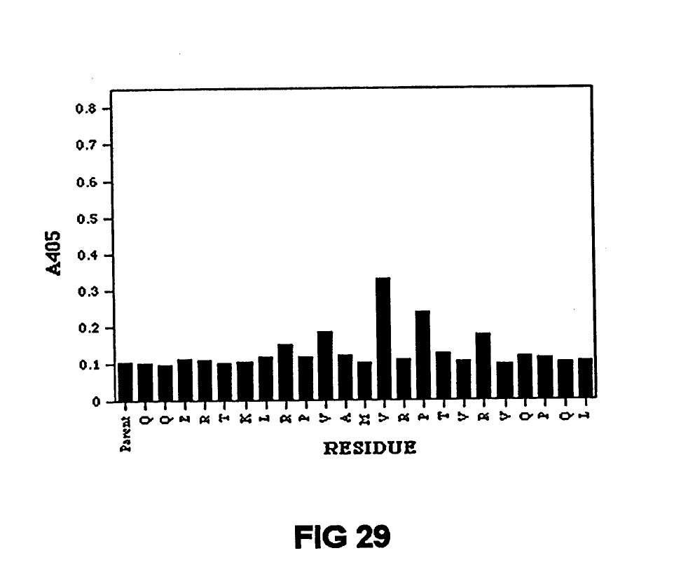

The initial screen of peptides yielded IC50 values of approximately 50-100 nM for wild-type peptide or peptides with ineffective mutations. For some alanine mutations, IC50 values increased to 200 to 500 nM, suggesting that those particular residues were important for interaction with the Siah molecule. These data are shown in FIG. 27. The absorbance values at the sensitive concentrations of 300 nM and 1000 nM peptide are plotted in FIGS. 28 and 29, to highlight those residues that, when substituted, have had marked effects on the potency of the competing peptide.

The results from the peptide screen support the importance of the valine 120 and proline 122 as predicted from the above sequence alignment, but suggest that other residues may be also be critical, including arginine 115, valine 117 and arginine 125.

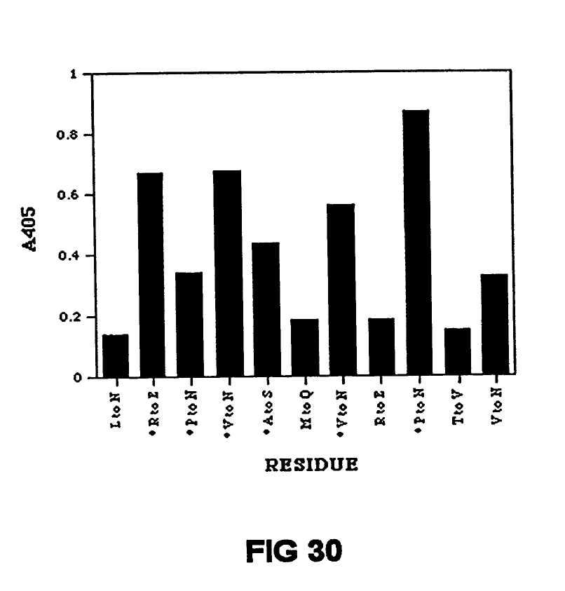

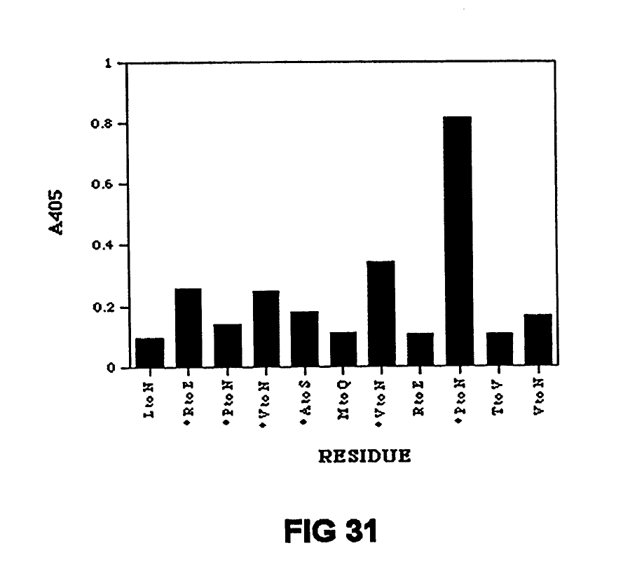

A second mutagenesis scan was performed, in which the residues from Leu114 through to Val124 were changed to residues of opposite characteristic ie. basic changed to acidic, hydrophobic changed to hydrophilic etc. (FIGS. 30 and 31). The results from this scan supported the results of the alanine mutagenesis scan, highlighting the same residues as being critical for interaction with Siah binding domain.

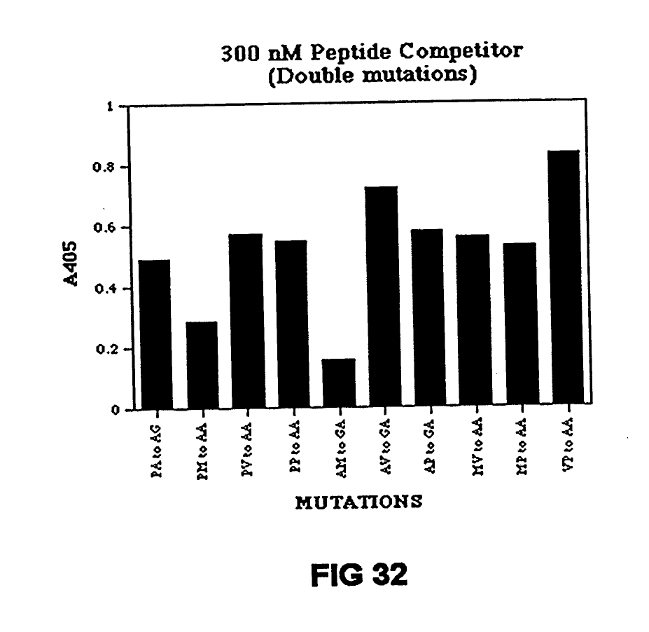

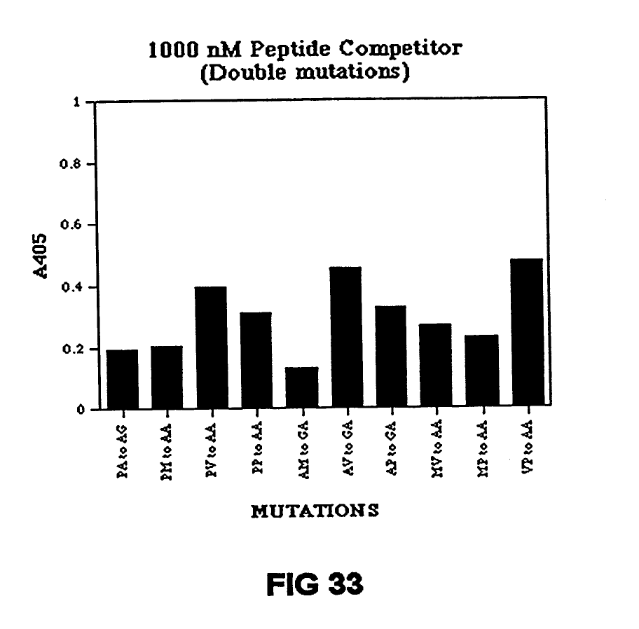

Further to the above studies, double mutants were synthesised, based on the proposed PXAXVXP motif predicted by sequence alignment, ie. Pro116/Ala118 to Ala116/Gly118, Pro116/Val120 to Ala116/Ala120, Pro116/Pro122 to Ala116/Ala122 etc. The results from this study (FIG. 32 and 33) support the results of the single mutation scans described above.

Overall, the mutagenesis scan has supported the proposal that Val120 and Pro122 in PHYL are important determinants for PHYL binding to Siah SBD, and to a lesser extent Pro116 and Ala118, as predicted from the sequence alignment with other interacting proteins. In addition, the mutagenesis scan has highlighted the importance of other residues within PHYL that may contribute to binding specificity including Arg115, Val117 and Arg125. The importance of the latter three residues was not apparent from the sequence alignment of binding partners, and they may contribute to the higher affinity binding observed between PHYL and Siah, compared to that observed for other proteins binding to Siah.

Accordingly, the present invention provides a ligand of the binding domain comprising the amino acid sequence VXP. Preferably, the ligand comprises the amino acid sequence PXAXVXP. More preferably the ligand comprise the amino acid sequence RPVAXVXPXXR.

In a preferred form of the invention the ligand has an affinity constant of from about 180 nM.

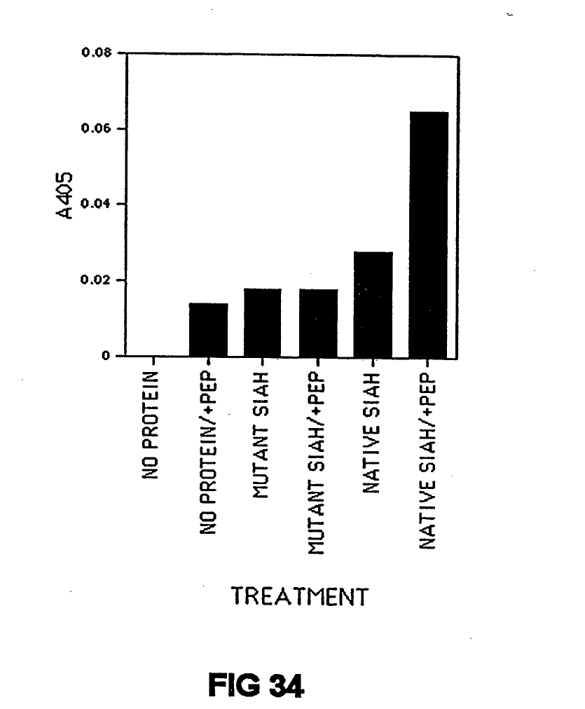

Following the identification of a Siah binding motif present in PHYL (RPVAXVXPXXR), a protein database search detected a number of proteins with conserved motifs, including plectin. The Applicants utilised a synthetic peptide corresponding to the sequence ASLQRVRRPVAMVMPARRTPHVQ in plectin to test binding to Siah in an ELISA-based assay, where the underlined sequence corresponds to the amino acids conserved with the PHYL motif. The peptide bound native Siah substrate-binding domain but not a mutant form (Leu211 to Arg) as shown in FIG. 34. The mutant form of Siah also does not bind PHYL. These results substantiate the ability of this motif to bind Siah substrate-binding domain and show the potential for the prediction of other binding partners based on sequence searches with the motif. Furthermore, as other Siah binding proteins are identified in this way it is predicted that the sequence of the optimal binding motif will be further refined.

Identification of Compounds that Bind to Siah Binding Domain.

The present invention also includes the use of the binding domain for identifying agonists and antagonists to the domain. Uses of these agonists and antagonists are also included in the scope of the present invention for the prevention and treatment of protein degrading diseases, tumour suppression, apoptosis, spermatogenesis or inflammation.

Accordingly the present invention provides a method of identifying a Siah binding domain agonist or antagonist the method including subjecting a potential binding domain agonist or antagonist to a binding domain or portion thereof of a Siah protein the domain being capable of binding to at least one protein, and determining the presence of an agonist or antagonist response.

Methods of determining the presence of an agonist or an antagonist response are known in the art and include competition assays, binding assays, protection assays and biosensor assays.

It is also possible to identify potential agonists and antagonists to a Siah binding domain by consideration of X-Ray crystallography data. Such data can determine which residues are on the surface of a protein molecule and therefore potentially able to interact with other molecules in solution. Accordingly, the present invention provides a method of identifying a compound which is capable of acting as an agonist or antagonist of binding to Siah, the method comprising the step of identifying a compound that has a conformation and polarity such that it interacts with an amino acid residue selected from the group consisting of:

Arg 233, Thr 235, Glu 237, His 230, Asn 228, Glu 226, Arg 224, Ala 222, Asn 276, Thr 278, Ser 280, Met 281, Cys 282, Phe 185, Thr 168, Leu 166, Val 164, Asp 162, Glu 161, Gly 160, Leu 158, Arg 231 and Cys 184, Trp 178, Lys 153, Thr 168, Thr 156, Phe 165, Val 179, Ser 154, Gly 174, Cys 130, Ser 134, Cys 135, His 152, Asp 177, Asp 169, Leu 172, Pro 173, Glu 194, Pro 131 and Leu 166, Arg 231, Arg 232, Glu 219, Lys 216, Arg 241, Glu 245, Arg 215, Asp 255, Asn 253, Leu 266, Glu 269, His 230, Gln 265, Met 252, Thr 249, Leu 120, Glu 119, Glu 116, Glu 113, Asn 270 and Ser 262; Asp 200, His 202, Tyr 199, Lys 198, Gln 204, Met 252, Ser 262, Asp 200, Gln 196, Asp 260, Gly 201, Thr 261 and Gln 203, Lys 114, Pro 97, Glu 118, His 111, Phe 96, Lys 99, Tyr 100, Arg 124, Glu 119, Ala 115 and Val 194; Cys 121, His 117, Cys 105, Ile 107, Leu 109, Phe 123, Leu 120 and Glu 122; of Siah1a or equivalent residues.

In a preferred method of the invention the compound interacts with at least two, more preferably three, or even more preferably four residues. It will be apparent to the skilled person that where the compound interacts with two or more residues, that those residues may be found on a single Siah monomer or distributed between two Siah monomers when Siah is in the form of a dimer.

Without wishing to be limited by theory, it is believed that these amino acid residues are involved in the binding of substrate and/or co-factor and/or interactor to the Siah protein. This information provides a rational basis for the development of compounds that can modulate the binding of substrates, co-factors and interactors to Siah, and therefore likely to have therapeutic value in the treatment of diseases relating to protein degradation.

The process of rational drug design requires no explanation or teaching for the skilled person, but a brief description of computational design is given here for the lay reader. Various computational analyses are necessary to determine whether a molecule is sufficiently complementary to the target moiety or structure to be useful as a pharmaceutical agent. Some of these analyses are discussed above. Such analyses may be carried out in current software applications, such as the Molecular Similarity application of QUANTA (Molecular Simulations Inc., Waltham, Mass.) version 3.3, and as described in the accompanying User's Guide, Volume 3 pages 134-135.

The Molecular Similarity application permits comparisons between different structures, different conformations of the same structure, and different parts of the same structure. The procedure used in Molecular Similarity to compare structures is divided into four steps:
1) load the structures to be compared;
2) define the atom equivalences in these structures;
3) perform a fitting operation; and
4) analyze the results.

Each structure is identified by a name. One structure is identified as the target (ie., the fixed structure); all remaining structures are working structures (ie., moving structures). When a rigid fitting method is used, the working structure is translated and rotated to obtain an optimum fit with the target structure. The fitting operation uses a least squares fitting algorithm which computes the optimum translation and rotation to be applied to the moving structure, such that the root mean square difference of the fit over the specified pairs of equivalent atom is an absolute minimum. This number, given in angstroms, is reported by QUANTA.

One skilled in the art may use one of several methods to screen chemical entities or fragments for their ability to associate with a target site. Again, these methods require no elucidation for the skilled person, but are described here for the benefit of the unskilled reader. The screening process begins by visual inspection of the target site on the computer screen, generated from a machine-readable storage medium. Selected fragments or chemical entitles may then be positioned in a variety of orientations, or docked, within that binding pocket as defined above. Docking may be accomplished using software such as Quanta and Sybyl, followed by energy minimization and molecular dynamics with standard molecular mechanics force fields, such as CHARMM and AMBER.

Specialized computer programs may also assist in the process of selected fragments or chemical entities. These include:
1. GRID (Goodford, 1985). GRID is available from Oxford University, Oxford, UK.
2. MCSS (Miranker et al., 1991). MCSS is available from Molecular Simulations, Burlington, Mass.
3. AUTODOCK (Goodsell, 1990). AUTODOCK is available from Scripps Research Institute, La Jolla, Calif.
4. DOCK (Kuntz, 1982). DOCK is available from University of California, San Francisco, Calif.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of the target compound or site. This would be followed by manual model building using software such as Quanta or Sybyl.

Useful programs to aid one of skill in the art in connecting the individual chemical entities or fragments include:
1. CAVEAT (Bartlett, 1989). CAVEAT is available from the University of California, Berkeley, Calif.
2. 3D Database systems such as MACCS-3D (MDL Information Systems, San Leandro, Calif.). This area is reviewed by Martin (1992).
3. HOOK (available from Molecular Simulations Burlington, Mass.).

As the skilled reader will already know, instead of proceeding to build a ligand for the target in a step-wise fashion, one fragment or chemical entity at a time as described above, target-binding compounds may be designed as a whole or de novo. These methods include:
1. LUDI (Bohm, 1992). LUDI is available from the Biosym Technologies, San Diego, Calif.
2. LEGEND (Nishibata, 1991). LEGEND is available from Molecular Simulations, Burlington, Mass.
3. LeapFrog (available from Tripos Associates, St. Louis. Mo.).

Other molecular modelling techniques may also be employed. See for example Cohen (1990). See also Navia (1992).

Once a compound has been designed or selected by such methods, the efficiency with which that compound can bind to a target site may be tested and optimized by computational evaluation. For example, an effective ligand will preferably demonstrate a relatively small difference in energy between its bound and free states, ie. a small deformation energy of binding. Thus the most efficient ligand should preferably be designed with a deformation energy of binding of not greater than about 10 kcal/mole, preferably, not greater than 7 kcal/mole. Ligands may interact with the target in more than one conformation that is similar in overall binding energy. In those cases, the deformation energy of binding is taken to be the difference between the energy of the free entity and the average energy of the conformations observed when the inhibitor binds to the protein.

An entity designed or selected as binding to a target may be further computionally optimized so that in its bound states it would preferably lack repulsive electrostatic interaction with the target enzyme. Such non-complementary (e.g., electrostatic) interactions include repulsive charge-charge, dipole-dipole and charge-dipole interactions. Specifically, the sum of all electrostatic interactions between the ligand and the target, when the ligand is bound to the target, preferably makes a neutral or favourable contribution to the enthalpy of binding.

Specific computer software is available in the art to evaluate compound deformation energy and electrostatic interaction. Examples of programs designed for such uses include: Gaussian 92, revision C (M. J. Frisch, Gaussian, Inc., Pittsburgh, Pa. COPYRGT. 1992]; AMBER, version 4.0 (P. A. Kollman, University of California at San Francisco, COPYRGT.1994]; QUANTA/CHARMM (Molecular Simulations, Inc., Burlington, Mass. COPYRGT.1994]; and Insight II/Discover (Biosysm Technologies Inc., San Diego, Calif. COPYRGT.1994). These programs may be implemented, for instance, using a Silicon Graphics workstation, IRIS 4D/35 or IBM RISC/6000 workstation model 550. Other hardware systems and software packages will be known to those skilled in the art.

Once the ligand has been optimally selected or designed, as described above, substitutions may then be made in some of its atoms or side groups in order to improve or modify its binding properties. Generally initial substitutions are conservative, ie., the replacement group will have approximately the same size, shape, hydrophobicity and charge as the original group. It should, of course, be understood that components known in the art to alter conformation should be avoided. Such substituted chemical compounds may then be analyzed for efficiency of fit to the desired target site by the same computer methods described in detail, above. Again, all these facts are familiar to the skilled person.

Another approach is the computational screening of small molecule data bases for chemical entities or compounds which can bind in whole, or in part, to a desired target. In this screening, the quality of fit of such entities to the binding site may be judged either by shape complementarity or by estimated interaction energy. (Meng, 1992).

The computational analysis and design of molecules, as well as software and computer systems therefor, are described in U.S. Pat. No. 5,978,740 which is included herein by reference, including specifically but not by way of limitation the computer system diagram described with reference to and illustrated in FIG. 3 thereof, as well as the data storage media diagram described with reference to and illustrated in FIGS. 4s and 5 thereof.

It is envisaged that the binding of ligands to relevant regions may promote enhance or, stabilise or mimic the binding of protein substrates, co-factors interactors or ligands of Siah. The preferential binding of ligands to Siah, preferably with an affinity in the order of 10⁻⁸M or better, may arise from enhanced stereochemical complementarity relative to naturally-occurring ligands.

Pursuant to the present invention, such stereochemical complementarity is characteristic of a molecule which matches intra-site surface residues lining the binding regions identified herein. By “match” we mean that the identified portions interact with the surface residues, for example, via hydrogen bonding or by enthalpy/entropy-reducing van der Waals interactions which promote desolvation of the biologically active compound within the site, in such a way that retention of the biologically active compound within the groove is energetically favoured.

It will be appreciated that it is not necessary that the complementarity between ligands and the substrate/ligand binding site extend over all residues lining the site in order to inhibit stabilise binding of the natural ligand. Accordingly, ligands which bind to some, but not all, of the residues lining the site are encompassed by the present invention.

In general, the design of a molecule possessing stereochemical complementarity can be accomplished by means of techniques which optimize, either chemically or geometrically, the “fit” between a molecule and a target receptor. Suitable such techniques are known in the art. (See Sheridan and Venkataraghavan, 1987; Goodford 1984; Beddell 1985; Hol, 1986; and Verlinde 1994, the respective contents of which are hereby incorporated by reference. See also Blundell 1987).

Thus there are two preferred approaches to designing a molecule according to the present invention, which complements the shape of the binding sites. In the first of these, the geometric approach, the number of internal degrees of freedom, and the corresponding local minima in the molecular conformation space, is reduced by considering only the geometric (hard-sphere) interactions of two rigid bodies, where one body (the active site) contains “pockets” or “grooves” which form binding sites for the second body (the complementing molecule, as ligand). The second approach entails an assessment of the interaction of different chemical groups (“probes”) with the active site at sample positions within and around the site, resulting in an array of energy values from which three-dimensional contour surfaces at selected energy levels can be generated.

The geometric approach is illustrated by Kuntz et al. (1982), the contents of which are hereby incorporated by reference, whose algorithm for ligand design is implemented in a commercial software package distributed by the Regents of the University of California and further described in a document, provided by the distributor, entitled “Overview of the DOCK Package, Version 1.0,”, the contents of which are hereby incorporated by reference. Pursuant to the Kuntz algorithm, the shape of the cavity represented by the substrate/ligand binding site is defined as a series of overlapping spheres of different radii. One or more extant databases of crystallographic data, such as the Cambridge Structural Database System maintained by Cambridge University (University Chemical Laboratory, Lensfield Road, Cambridge CB2 1EW, U.K.) and the Protein Data Bank maintained by the Research Collaboratory for Structural Bioinformatics (RCSB; http//www.rcsb.org/index.html) is then searched for molecules which approximate the shape thus defined.

Molecules identified in this way, on the basis of geometric parameters, can then be modified to satisfy criteria associated with chemical complementarity, such as hydrogen bonding, ionic interactions and van der Waals interactions.

The chemical-probe approach to ligand design is described, for example, by Goodford (1985), the contents of which are hereby incorporated by reference, and is implemented in several commercial software packages, such as GRID (product of Molecular Discovery Ltd., West Way House, Elms Parade, Oxford OX2 9LL, U.K.). Pursuant to this approach, the chemical prerequisites for a site-complementing molecule are identified at the outset, by probing the substrate/ligand binding site with different chemical probes, e.g., water, a methyl group, an amine nitrogen, a carboxyl oxygen, and a hydroxyl. Favoured sites for interaction between the active site and each probe are thus determined, and from the resulting three-dimensional pattern of such sites a putative complementary molecule can be generated.

Programs suitable for searching three-dimensional databases to identify molecules bearing a desired pharmacophore include: MACCS-3D and ISIS/3D (Molecular Design Ltd., San Leandro, Calif.), ChemDBS-3D (Chemical Design Ltd., Oxford, U.K.), and Sybyl/3DB Unity (Tripos Associates, St. Louis, Mo.).

Programs suitable for pharmacophore selection and design include: DISCO (Abbott Laboratories, Abbott Park, Ill.), Catalyst (Bio-CAD Corp., Mountain View, Calif.), and ChemDBS-3D (Chemical Design Ltd., Oxford, U.K.).

Databases of chemical structures are available from a number of sources including Cambridge Crystallographic Data Centre (Cambridge, U.K.) and Chemical Abstracts Service (Columbus, Ohio).

De novo design programs include Ludi (Biosym Technologies Inc., San Diego, Calif.), Sybyl (Tripos Associates) and Aladdin (Daylight Chemical Information Systems, Irvine, Calif.).

Those skilled in the art will recognize that the design of a mimetic compound may require slight structural alteration or adjustment of a chemical structure designed or identified using the methods of the invention. This aspect of the invention may be implemented in hardware or software, or a combination of both. However, the invention is preferably implemented in computer programs executing on programmable computers each comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object-oriented programming language to communicate with a computer system. However, the programs can be implemented in assembly or machine language, if desired. In any case, the language may be compiled or interpreted language.

Each such computer program is preferably stored on a storage medium or device (e.g., ROM or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

Compounds identified by the methods of the present invention may be assessed by a number of in vitro and in vivo assays. For example, binding affinity for candidate ligands may be measured using biosensor technology. A compound identified by the methods described herein may be subsequently subjected to in vitro and/or in vivo testing for ability to act as an antagonist/agonist of binding of natural substrates and/or co-factors and/or interactors of Siah. Useful compounds will have the ability to interact with the binding domain of Siah in such a way that the binding of a natural substrate and/or co-factor and/or interactor is either enhanced or reduced.

Preferably the compound binds to at least two, more preferably at least three, and even more preferably four of the groups of amino acid residues identified above. Even more preferably the compound has the ability to penetrate the cell membrane.

It will be clearly understood that the term “identifying” encompasses either designing a new compound, or selecting a compound from a group or library of previously known compounds.

The term “agonist” refers to:
a) a compound which has a conformation and polarity such that the compound itself binds to the binding domain of Siah;
b) a compound which has a conformation and polarity such that the compound binds to the binding domain of Siah at a site other than the binding site, and this enhances or stabilises the binding of protein substrate and/or co-factor and/or interactor and/or ligand to Siah; or
c) a compound which has a conformation and polarity such that the compound binds to Siah at a site other than the binding site, in which the binding has no effect on substrate, co-factor, or interactor/ligand binding but induces an effect the same as or similar to one which is induced by binding of substrate and/or co-factor and/or interactor to Siah.

It will be appreciated that a compound may have more than one of these abilities.

The agonist and antagonist compounds of the present invention are not limited to antibodies reactive to the protein-binding domain or any portions thereof and which compete with the binding of substrate and/or co-factor and/or interactor. Other compounds including small molecules or synthetic or natural chemical compounds capable of competing with the binding of a substrate and/or co-factor and/or interactor to the binding domain or any portion thereof are also included in the present invention.

Furthermore, it is contemplated that compounds which inhibit Siah dimerisation could be predicted and screened for given the structure of the dimerisation interface. Such compounds may include the agonists and antagonists identified above. These may be further identified by testing for competitionassays which inhibit or improve dimerisation of the Siah molecules.

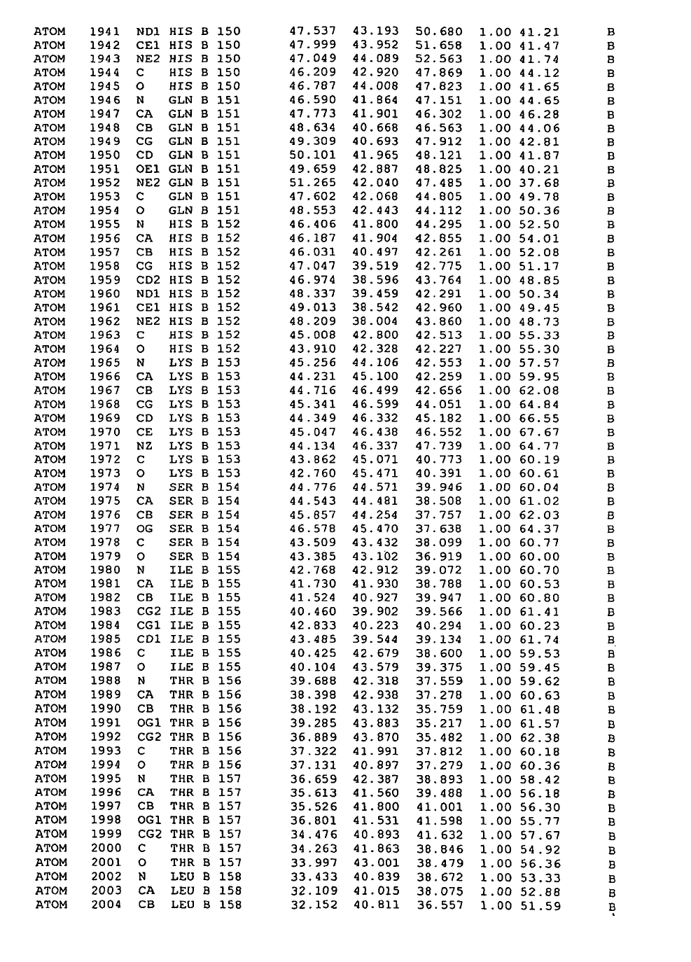

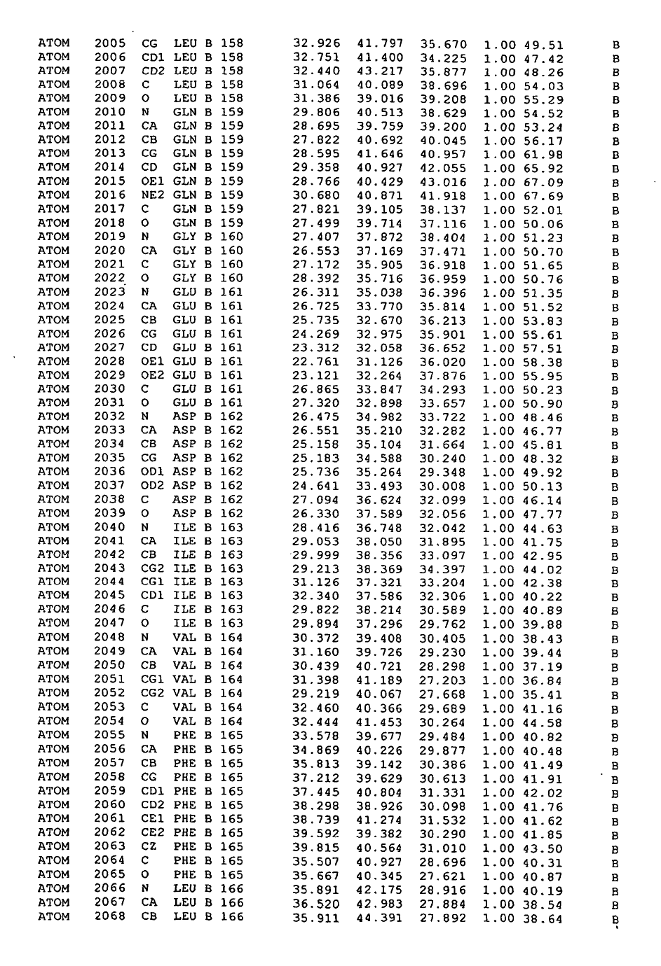

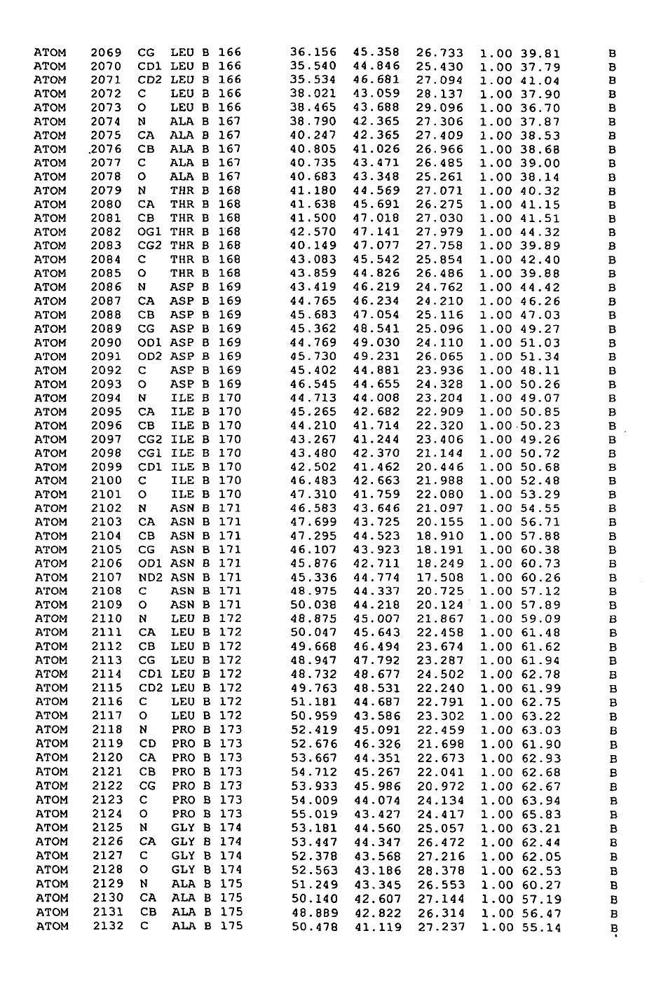

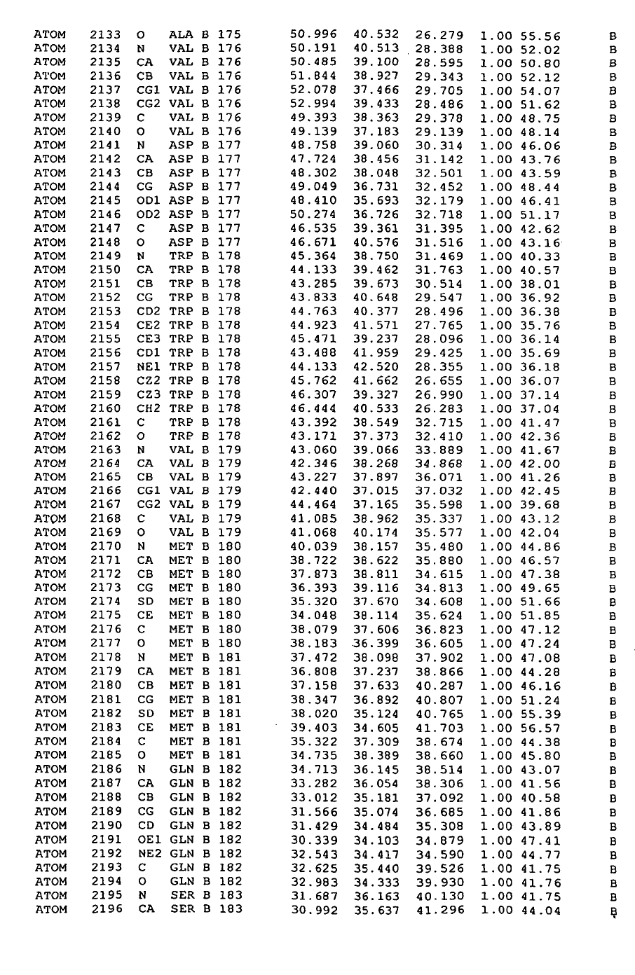

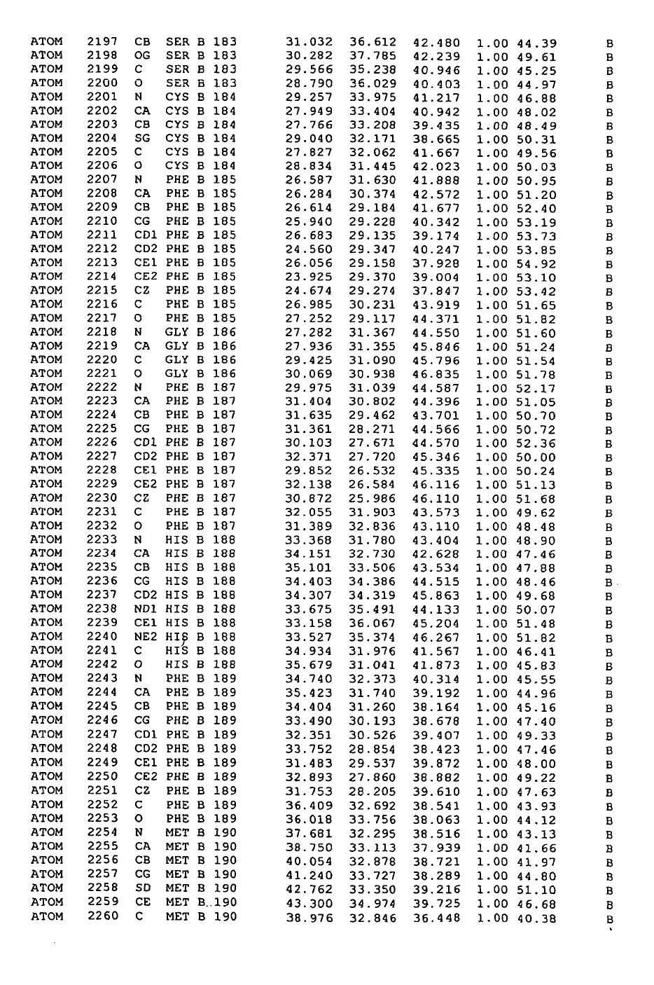

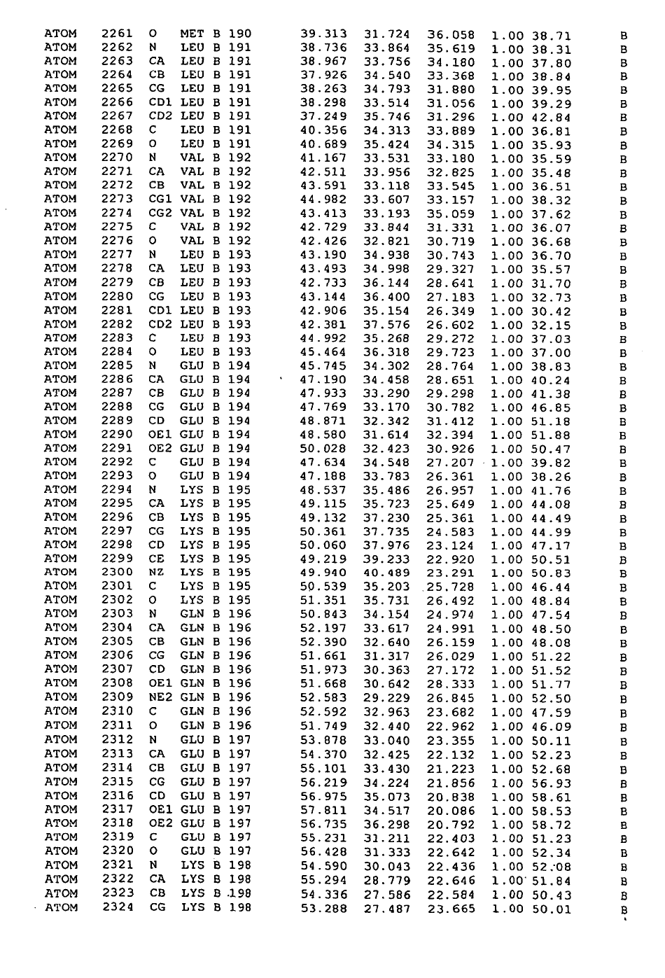

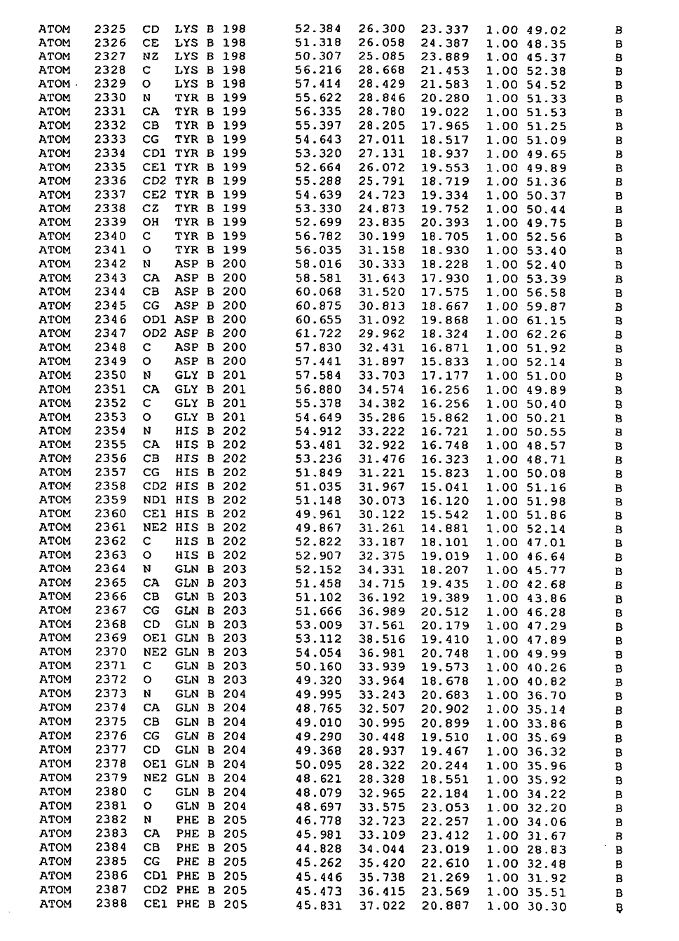

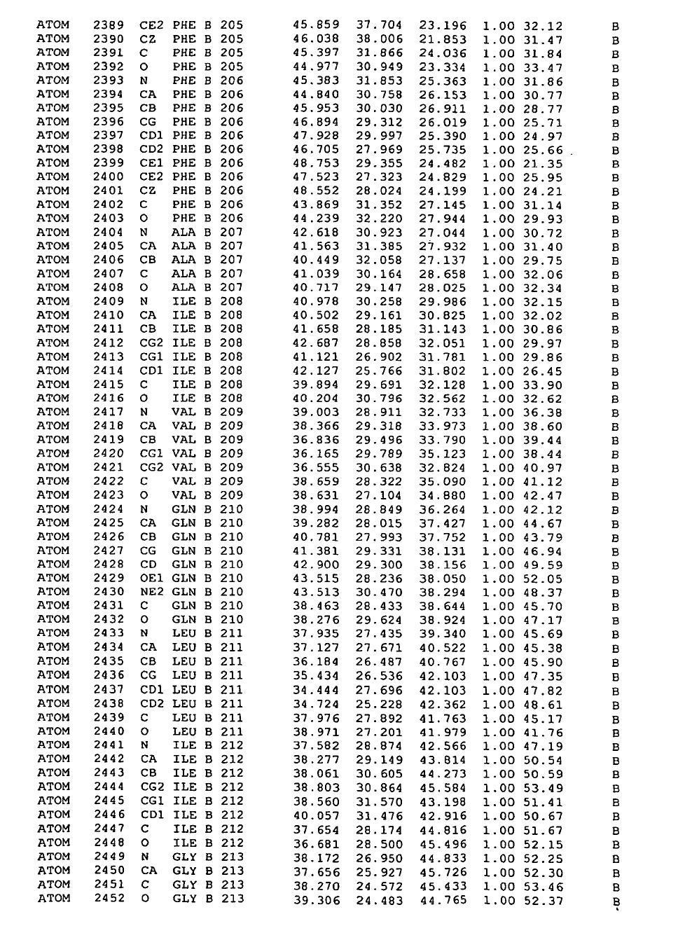

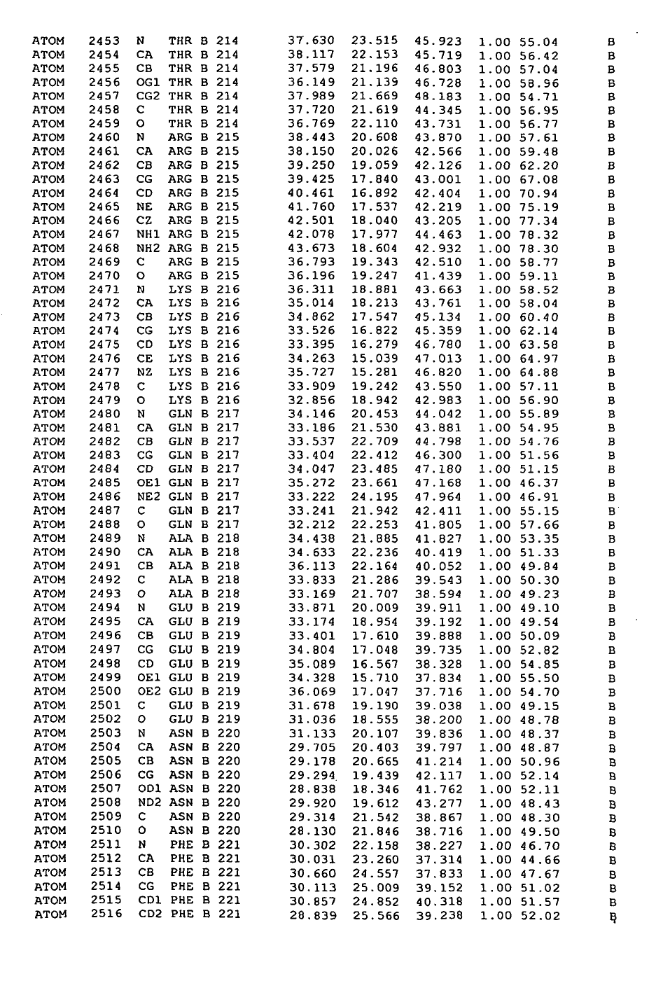

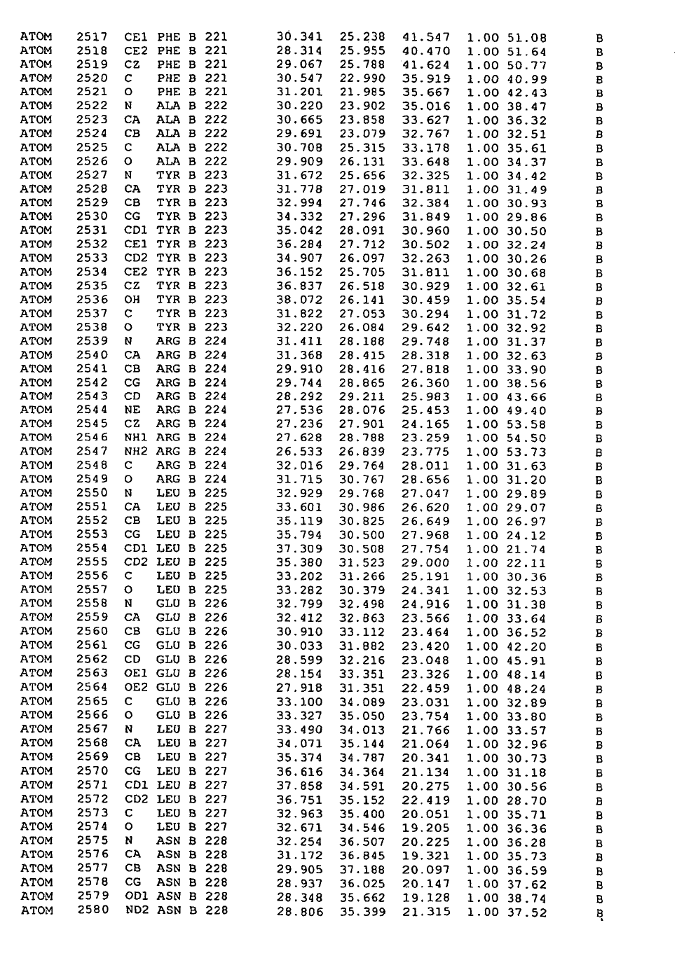

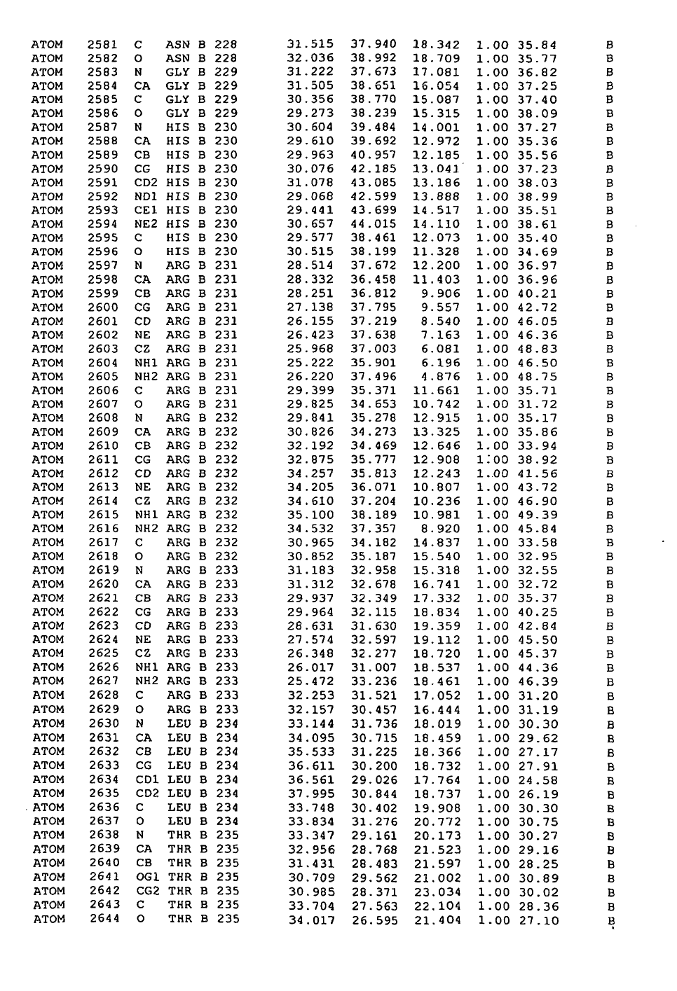

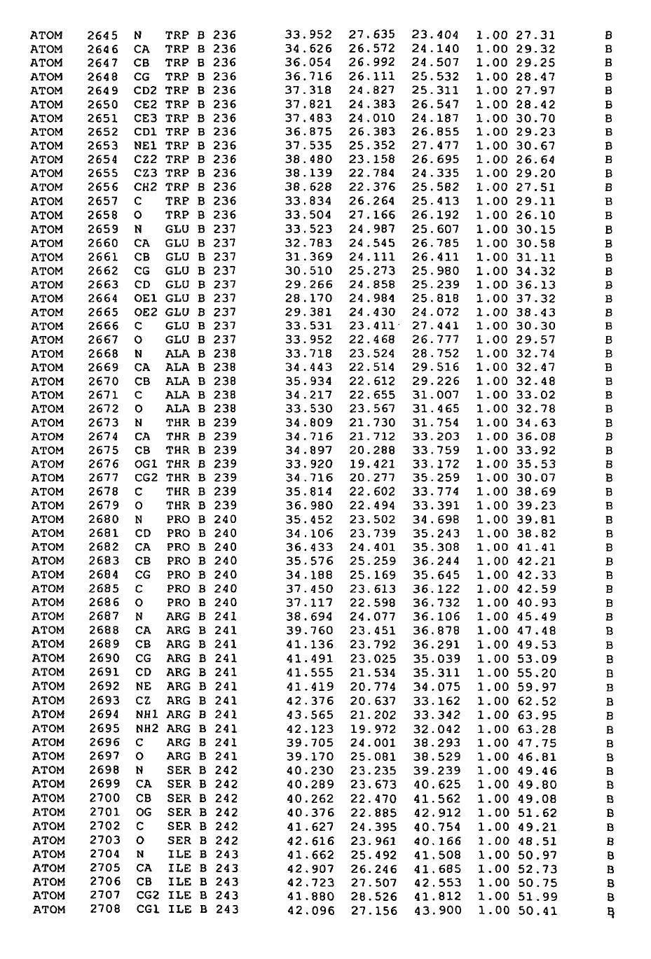

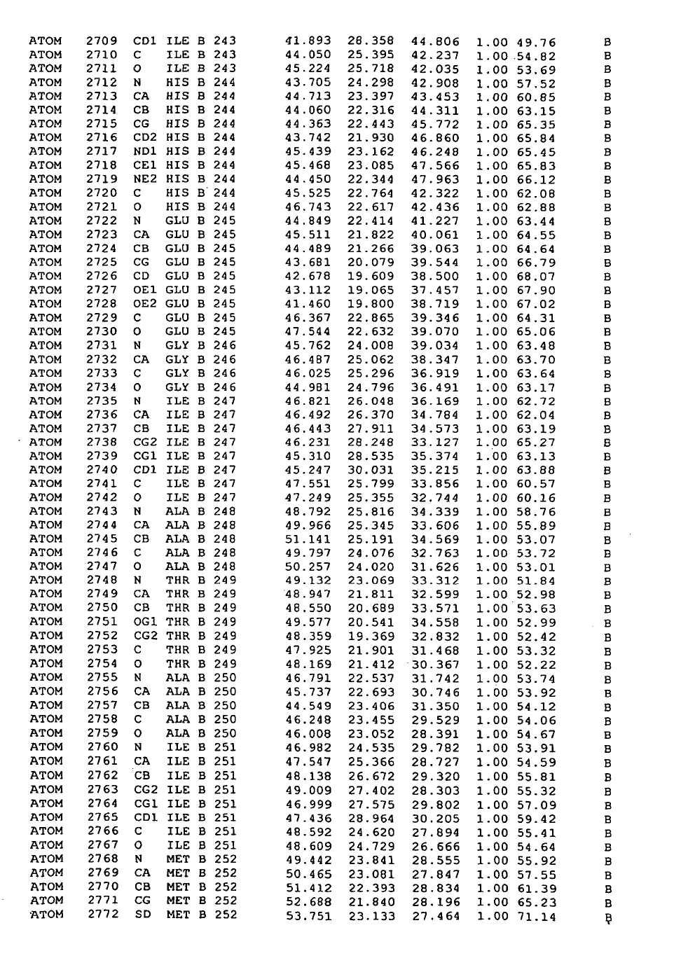

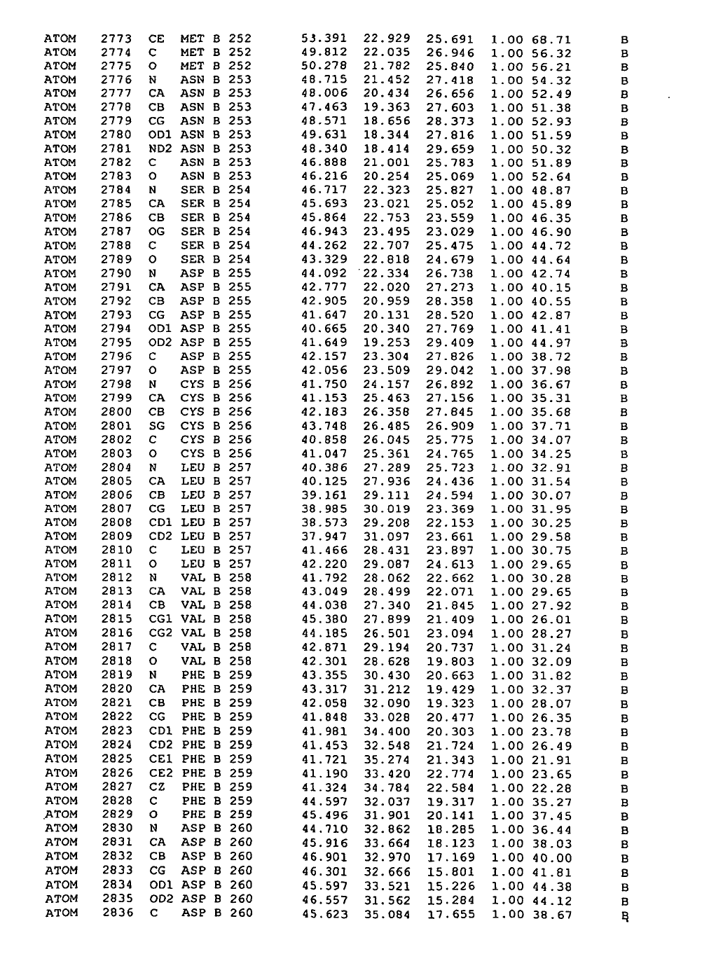

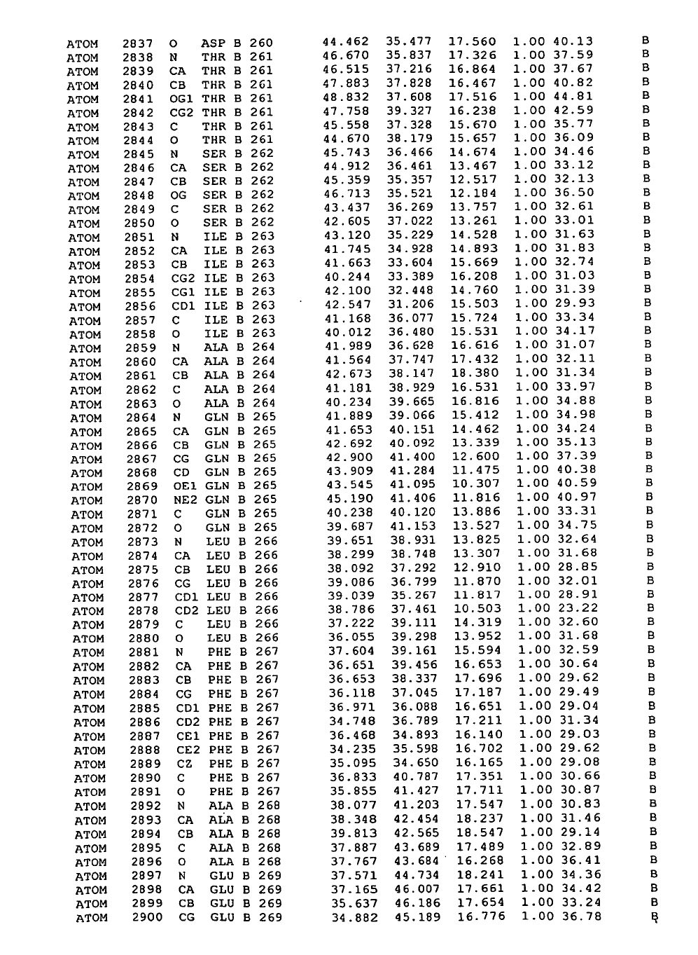

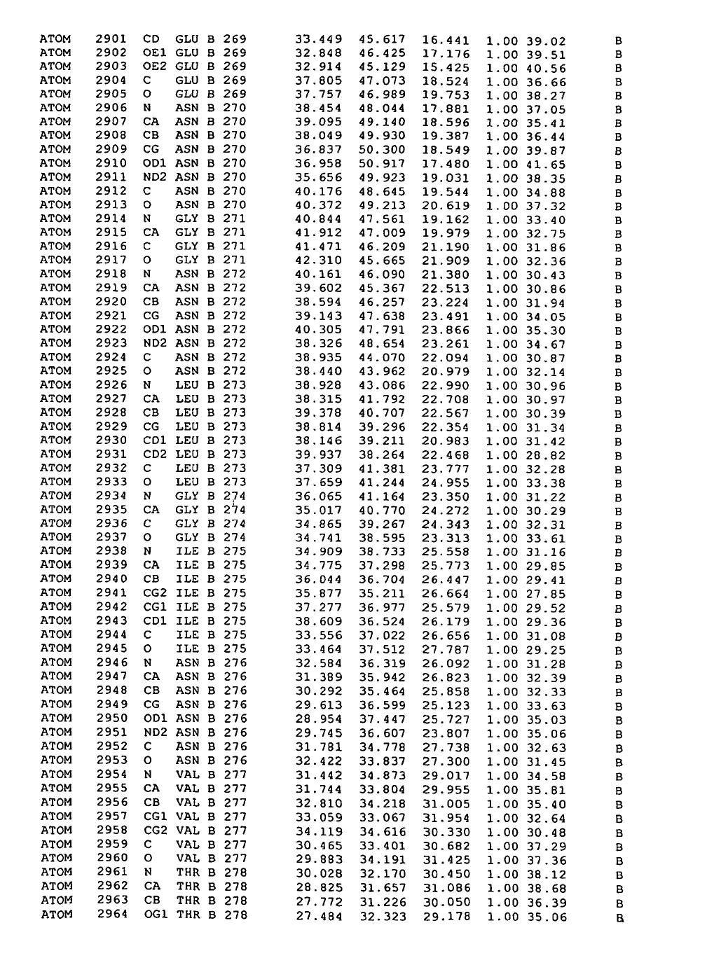

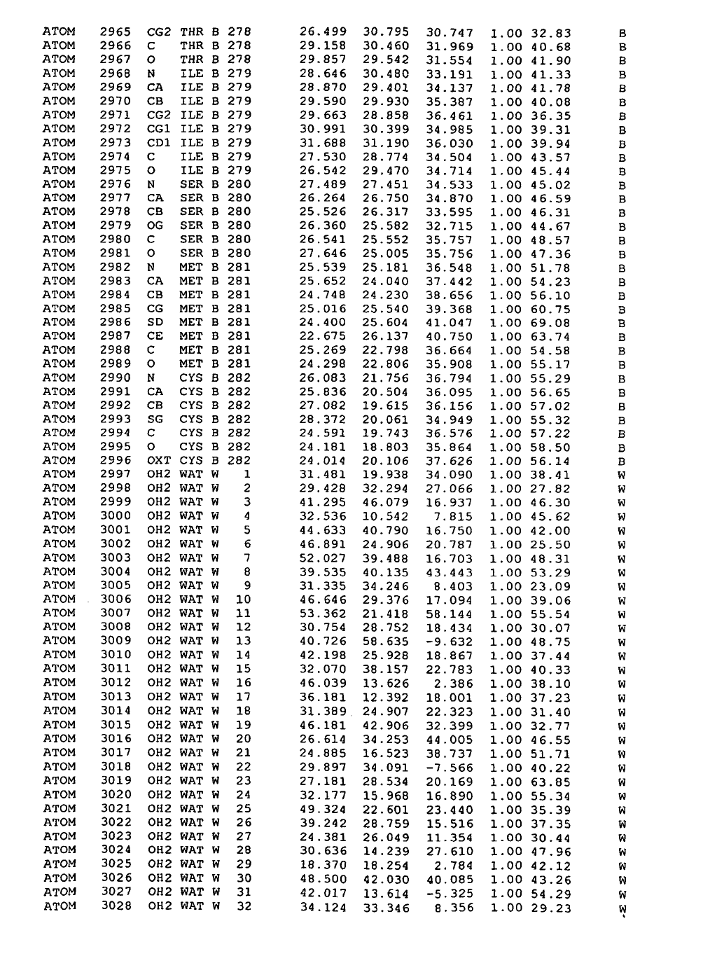

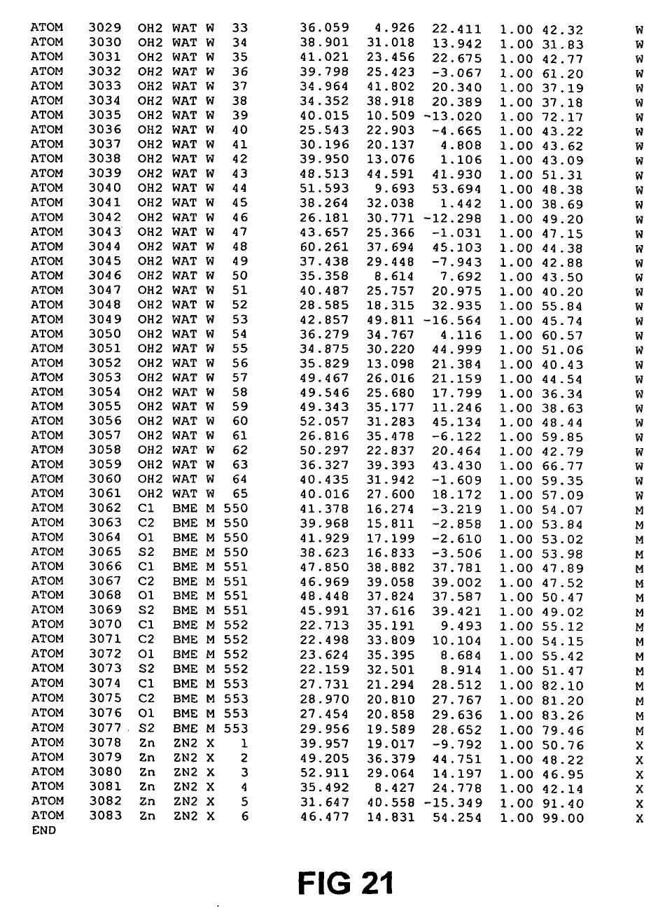

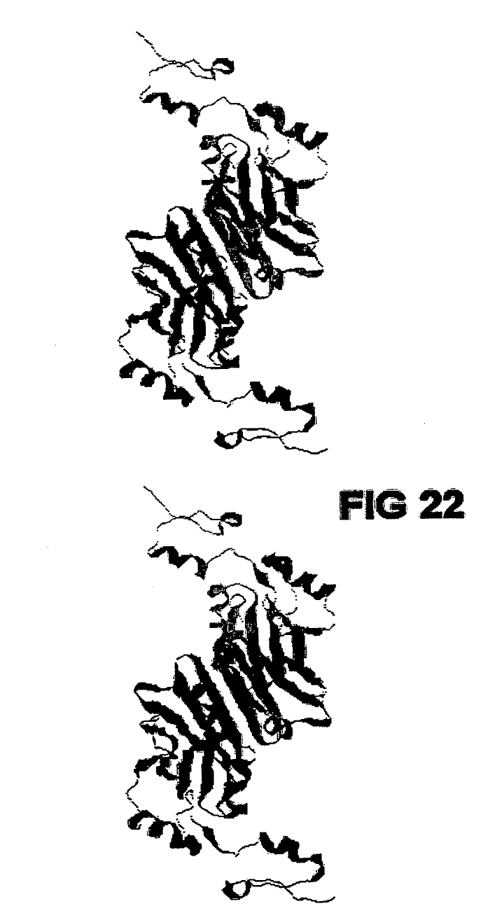

In a yet a further aspect, the present invention provides a computer-assisted method of identifying compounds potentially able to bind to the binding domain of Siah using a programmed computer the method comprising the steps of:
(a) inputting into the programmed computer data comprising the atomic coordinates of the Siah binding domain, as shown in FIG. 21, corresponding to a binding site defined by amino acid residues
   Arg 233, Thr 235, Glu 237, His 230, Asn 228, Glu 226, Arg 224, Ala 222, Asn 276, Thr 278, Ser 280, Met 281, Cys 282, Phe 185, Thr 168, Leu 166, Val 164, Asp 162, Glu 161, Gly 160, Leu 158, Arg 231 and Cys 184, Trp 178, Lys 153, Thr 168, Thr 156, Phe 165, Val 179, Ser 154, Gly 174, Cys 130, Ser 134, Cys 135, His 152, Asp 177, Asp 169, Leu 172, Pro 173, Glu 194, Pro 131 and Leu 166, Arg 231, Arg 232, Glu 219, Lys 216, Arg 241, Glu 245, Arg 215, Asp 255, Asn 253, Leu 266, Glu 269, His 230, Gln 265, Met 252, Thr 249, Leu 120, Glu 119, Glu 116, Glu 113, Asn 270 and Ser 262, Asp 200, His 202, Tyr 199, Lys 198, Gln 204, Met 252, Ser 262, Asp 200, Gln 196, Asp 260, Gly 201, Thr 261 and Gln 203, Lys 114, Pro 97, Glu 118, His 111, Phe 96, Lys 99, Tyr 100, Arg 124, Glu 119, Ala 115 and Val 194, Cys 121, His 117, Cys 105, Ile 107, Leu 109, Phe 123, Leu 120 and Glu 122;
(b) generating, using computer methods, a set of atomic coordinates of a structure that possesses stereochemical complementarity to the atomic coordinates defined in (a) or a subset thereof, thereby generating a criteria data set;
(c) comparing, using the processor, the criteria data set to a computer database of chemical structures;
(d) selecting from the database, using computer methods, chemical structures which are similar to a portion of the criteria data set; and
(e) outputting the selected chemical structures which are similar to a portion of the criteria data set.

Preferably the method further comprises the step of obtaining a compound with a chemical structure selected in steps (d) and (e), and testing the compound for the ability to bind to a Siah protein.

In another aspect the invention provides a computer or a software component thereof for producing a three-dimensional representation of a molecule or molecular complex, which comprises a three-dimensional representation of a homologue of the molecule or molecular complex, in which the homologue comprises a domain that has a root mean square deviation from the backbone atoms of the amino acids of not more than 1.5 Å, in which the computer comprises:
(a) a machine-readable data storage medium comprising a data storage material encoded with machine-readable data, wherein the data comprises the structure coordinates, as shown in FIG. 21, of:
   Arg 233, Thr 235, Glu 237, His 230, Asn 228, Glu 226, Arg 224, Ala 222, Asn 276, Thr 278, Ser 280, Met 281, Cys 282, Phe 185, Thr 168, Leu 166, Val 164, Asp 162, Glu 161, Gly 160, Leu 158, Arg 231 and Cys 184, Trp 178, Lys 153, Thr 168, Thr 156, Phe 165, Val 179, Ser 154, Gly 174, Cys 130, Ser 134, Cys 135, His 152, Asp 177, Asp 169, Leu 172, Pro 173, Glu 194, Pro 131 and Leu 166, Arg 231, Arg 232, Glu 219, Lys 216, Arg 241, Glu 245, Arg 215, Asp 255, Asn 253, Leu 266, Glu 269, His 230, Gln 265, Met 252, Thr 249, Leu 120, Glu 119, Glu 116, Glu 113, Asn 270 and Ser 262, Asp 200, His 202, Tyr 199, Lys 198, Gln 204, Met 252, Ser 262, Asp 200, Gln 196, Asp 260, Gly 201, Thr 261 and Gln 203, Lys 114, Pro 97, Glu 118, His 111, Phe 96, Lys 99, Tyr 100, Arg 124, Glu 119, Ala 115 and Val 194, Cys 121, His 117, Cys 105, Ile 107, Leu 109, Phe 123, Leu 120 and Glu 122;
(b) a working memory for storing instructions for processing the machine-readable data;
(c) a central-processing unit coupled to the working memory and to the machine-readable data storage medium for processing the machine-readable data into the three-dimensional representation ; and
(d) a display coupled to the central-processing unit for displaying the three-dimensional representation.

In one class of embodiments, the three-dimensional representation is of a molecule or molecular complex defined by the set of structure coordinates set out in FIG. 21, or wherein the three-dimensional representation is of a homologue of the molecule or molecular complex, the homologue having a root mean square deviation from the backbone atoms of the amino acids of not more than 1.5 Å.

An additional aspect of the invention provides a computer or a software component thereof for determining at least a portion of the structure coordinates corresponding to a three-dimensional structure of a molecule or molecular complex, in which the computer comprises:
(a) a machine-readable data storage medium comprising a data storage material encoded with machine-readable data, in which the data comprises at least a portion of the structural coordinates according to FIG. 21;
(b) a machine-readable data storage medium comprising a data storage material encoded with machine-readable data, wherein the data comprise crystallographic data of the molecule or molecular complex;
(c) a working memory for storing instructions for processing the machine-readable data of (a) and (b);
(d) a central-processing unit coupled to the working memory and to the machine-readable data storage medium of (a) and (b) for performing a transformation of the machine readable data of (a) and for processing the machine-readable data of (b) into structure coordinates; and
(e) a display coupled to the central-processing unit for displaying the structure coordinates of the molecule or molecular complex.

In a further aspect the invention provides a compound able to act as an antagonist or agonist of the binding of protein substrates, co-factors, interactors or ligands to the binding domain of Siah, wherein the compound is identified by a method described herein.

In a preferred form of the invention the compound has a conformation and polarity such that it interacts at least one amino acid residue selected from the group consisting of:
Arg 233, Thr 235, Glu 237, His 230, Asn 228, Glu 226, Arg 224, Ala 222, Asn 276, Thr 278, Ser 280, Met 281, Cys 282, Phe 185, Thr 168, Leu 166, Val 164, Asp 162, Glu 161, Gly 160, Leu 158, Arg 231 and Cys 184, Trp 178, Lys 153, Thr 168, Thr 156, Phe 165, Val 179, Ser 154, Gly 174, Cys 130, Ser 134, Cys 135, His 152, Asp 177, Asp 169, Leu 172, Pro 173, Glu 194, Pro 131 and Leu 166, Arg 231, Arg 232, Glu 219, Lys 216, Arg 241, Glu 245, Arg 215, Asp 255, Asn 253, Leu 266, Glu 269, His 230, Gln 265, Met 252, Thr 249, Leu 120, Glu 119, Glu 116, Glu 113, Asn 270 and Ser 262, Asp 200, His 202, Tyr 199, Lys 198, Gln 204, Met 252, Ser 262, Asp 200, Gln 196, Asp 260, Gly 201, Thr 261 and Gln 203, Lys 114, Pro 97, Glu 118, His 111, Phe 96, Lys 99, Tyr 100, Arg 124, Glu 119, Ala 115 and Val 194, Cys 121, His 117, Cys 105, Ile 107, Leu 109, Phe 123, Leu 120 and Glu 122; of Siah1a or equivalent residues.

Preferably the compound interacts with at least two amino acid residues, more preferably with at least three amino acid residues. Even more preferably the compound interacts with at least two, and still more preferably at least four amino acid residues.

It is preferred that the compound has a high affinity for the selected target site. For in silico screening using computer modelling systems, the affinity constant is preferably ≦1 μM, more preferably ≦1 nM.

It will be clearly understood that pharmaceutically acceptable salts, derivatives and esters of these compounds are also within the scope of the invention. Accordingly, a further aspect of the invention provides a composition comprising a compound according to the invention, together with a pharmaceutically-acceptable carrier. It will be appreciated that the composition may comprise two or more compounds according to the invention.

The compounds of the invention may be formulated into pharmaceutical compositions, and administered in therapeutically effective doses. By “therapeutically effective dose” is meant a dose which results in at least partial alleviation of the symptoms or pathophysiological effects of the disease. The appropriate dose will be ascertainable by one skilled in the art using known techniques.

The pharmaceutical compositions may be administered in a number of ways, including, but not limited to, orally, subcutaneously, intravenously, intraperitoneally and intranasally.

The dosage to be used will depend on the nature and severity of the condition to be treated, and will be at the discretion of the attending physician or veterinarian. The most suitable dosage for a specific condition can be determined using normal clinical trial procedures.

While it is particularly contemplated that the compounds of the invention are suitable for use in medical treatment of humans, they are also applicable to veterinary treatment, including treatment of companion animals such as dogs and cats, and domestic animals such as horses, cattle and sheep, or zoo animals such as felids, canids, bovids, and ungulates.

Methods and pharmaceutical carriers for preparation of pharmaceutical compositions are well known in the art, as set out in textbooks such as Remington's Pharmaceutical Sciences, 19th Edition, Mack Publishing Company, Easton, Pa., USA.

The compounds and compositions of the invention may be administered by any suitable route, and the person skilled in the art will readily be able to determine the most suitable route and dose for the condition to be treated. Dosage will be at the discretion of the attendant physician or veterinarian, and will depend on the nature and state of the condition to be treated, the age and general state of health of the subject to be treated, the route of administration, and any previous treatment which may have been administered.

The carrier or diluent, and other excipients, will depend on the route of administration, and again the person skilled in the art will readily be able to determine the most suitable formulation for each particular case.

In another aspect, there is provided a method of treating or preventing a disease relating to abnormal protein degradation, the method comprising administering to a subject in need thereof an effective amount of a composition of the present invention.

Preferably the compounds are used to treat or prevent conditions such as cancer, inflammation, infertility, pathological immune responses, neurological disorders, disease relating to apoptosis, disease relating to NFκB signalling. It will be apparent to the skilled person that the compositions could be used for other related diseases not expressly defined herein.

Because Siah has been reported to be upregulated in a p53-dependent manner, it has been termed a tumour suppressor. Some reports suggest that Siah is pro-apoptotic, whilst others suggest that Siah causes growth arrest rather than apoptosis. Indirect evidence linking Siah and apoptosis involves the upregulation of Siah1 at the tips of villi in the small intestine, a region of natural apoptosis.

The present invention contemplates that Siah antagonists will be useful as a therapeutic in breast cancer. Tamoxifen is an anti-estrogen that is used in the treatment of estrogen receptor positive breast cancer. By controlling the balance of coactivators and corepressors of estrogen receptor responsive genes (Smith et al., 1997), Siah will render cancer cells resistant to tamoxifen (a phenomenon observed in some breast cancers). Blocking Siah function will be useful in this context as Siah has been shown to be involved in degradation of the estrogen receptor co-repressor N—CoR (Zhang et. al. 1998), a build-up of which (due to the absence of Siah activity) would repress estrogen receptor transcriptional responses. This can be tested by modulating Siah levels or activity in cancer cell lines known to be tamoxifen-resistant, either by introduction of Siah dominant negative protein fragments or Siah antagonists, thus rendering the cells sensitive to tamoxifen treatment. Siah antagonists may be synthetic peptides fused to cargo delivery peptides such as Drosophila antennapedia homeodomain peptide (Fischer et al. 2001).

Siah is involved in inflammation and immune responses via the IL-1 signalling pathway. It has been shown that transfection of Siah1 can stimulate the activation of NF-κB after stimulation of cells with IL-1. However, Siah2 in this same setting inhibits the NF-κB production, indicative of functional differences between Siah1 and Siah2. This response seems to be mediated via an interaction between Siah and PW1, a protein capable of binding to TRAF TNF-receptor associated factor. Interestingly, the binding domain described in this application, when transfected, also stimulated greatly the NF-κB production.

Another aspect of the present invention is a method of assaying ubiquitination the method comprising the steps of providing a source of Siah, E1 enzyme, E2 enzyme, a substrate, ubiquitin, and ATP, and wherein the substrate is ubiquinated if it is capable of binding to the substrate binding site of Siah.

Another aspect provides a method of assaying NFκB activation the method comprising the steps of providing a cell transfected to express Siah and measuring the activation of NFκB. Preferably activation is measured using a reporter construct assay utilizing the expression of luciferase.

Activation of NFκB may be measured using a standard proprietary Promega reporter construct assay, utilizing the expression of firefly luciferase and quantitation in a luminometer. The NFκB DNA recognition sequence linked to the DNA for luciferase expression may be transiently transfected into mammalian cells human kidney 293 cells along with constructs of Siah1a and PW1, so that increased levels of NFκB would stimulate luciferase expression. After cell lysis, substrate is added to the lysate and light-emitting product is measured on a luminometer, giving a readout proportional to the amount of NFκB released in response to the Siah1a and PW1 transfected. Agonists and antagonists of the NFκB response may be screened using this assay. In a further aspect there is provided a crystalline form of Siah. Preferably the crystalline form of Siah has at least one atomic co-ordinate within 0.5 Å of an atomic co-ordinate defined in FIG. 21.

Provision of a crystalline form of the binding domain has allowed the Applicants to determine the structure of the binding domain. This structure allows for the rational design of compounds, agonists and antagonists which have the ability to modulate the binding of a substrate, co-factor or interactor to the binding domain, dimerisation domain, or zinc binding domain.

The present invention will now be more fully described with reference to the following examples. It should be understood, however, that the description following is illustrative only and should not be taken in any way as a restriction on the generality of the invention described above.

### EXAMPLES

### Example 1

### Expression and Purification of Siah1a binding Domain

Applicants expressed the Siah1a binding domain, residues 80-282, in E. coli BL21DE3 cells as a fusion partner of maltose-binding protein MBP using the pMalC2 plasmid (NEB). The C-terminal fragment was cloned into the pMalC2 plasmid using a natural BamH1 site in the Siah1a DNA and a HindIII site engineered at the C-terminus.

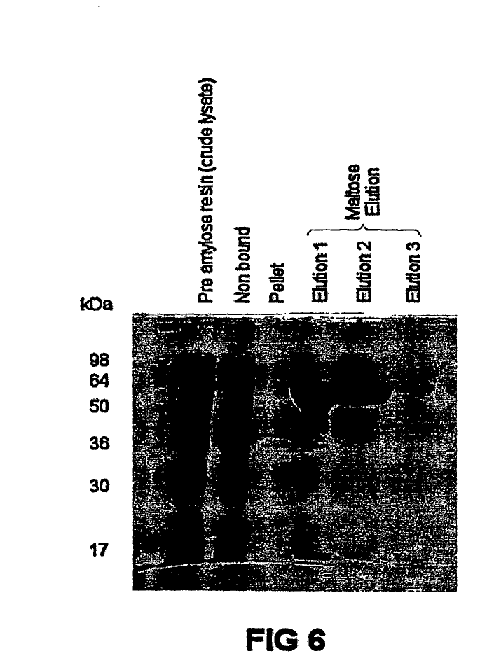

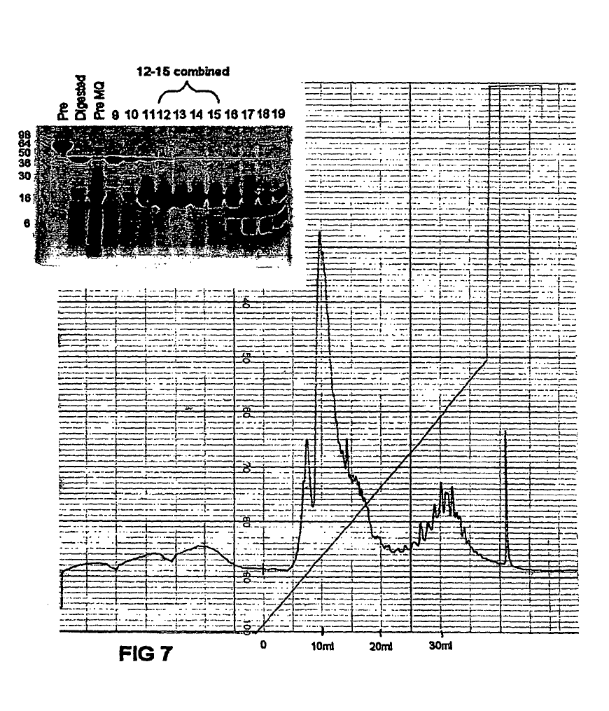

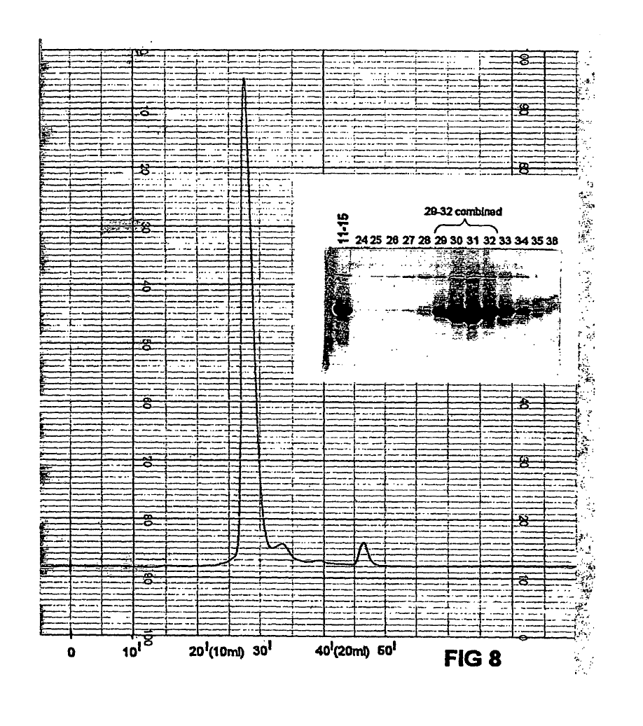

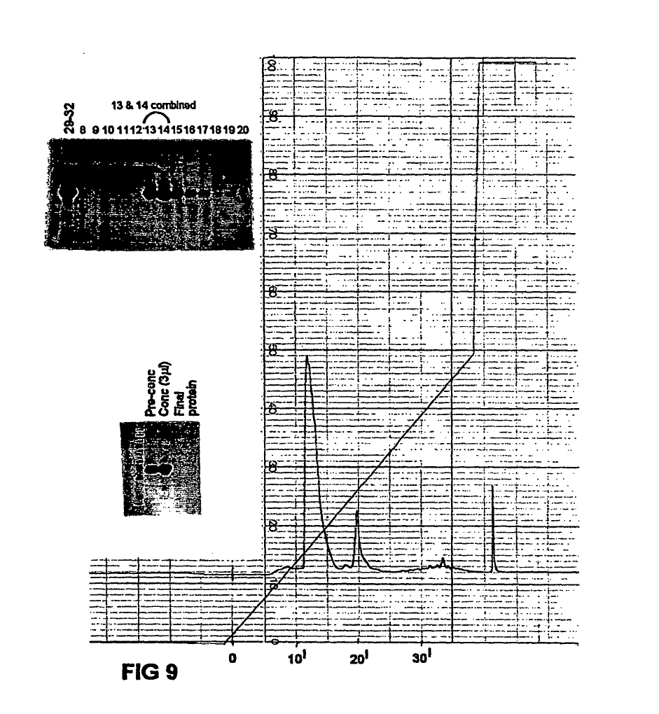

Expression was induced with 0.5 mM IPTG at 0.6-0.9 OD₆₀₀ for 5 h at 22° C. Expression at 37° C. produced misfolded protein which gave very poor yields during purification. The fusion protein was purified by amylose affinity chromatography in 50 mM Tris-HCl pH 8.0, 200 mM NaCl, 15 mM 2-ME as described by the manufacturer (NEB). The fusion protein was eluted from the affinity column with 10 mM maltose in the above buffer (FIG. 6), then cleaved in solution with trypsin, 10 mg trypsin per 150 mg fusion protein, 5 min, 0° C., terminated by addition of 1 mM PMSF and 100 μg/ml leupeptin to generate a stable Siah fragment (FIG. 7 inset). The Siah fragment was enriched by ammonium sulfate precipitation (50% saturation), which precipitated most of the Siah fragment but very little of the contaminating MBP. After centrifugation at 20,000×g for 15 min, the precipitated Siah fragment was redissolved in 50 mM Tris-HCl pH 8.0, 200 mM NaCl, 15 mM 2-ME, 0.2 μm filtered, then desalted on PD-10 columns (Pharmacia) before loading onto anion exchange chromatography Mono Q (Pharmacia). Protein was eluted at 1 ml/min with a 0-0.4 M NaCl gradient over 30 min (FIG. 7). Fractions of 1 ml were collected and assessed for protein purity by analytical SDS-polyacrylamide gel electrophoresis (SDS-PAGE) (FIG. 7 inset). Appropriate fractions were pooled and concentrated to 0.3-0.4 ml for size exclusion chromatography on Superdex 200 HR 1.0×30 cm in 50 mM Tris-HCl, 200 mM NaCl, 15 mM 2-ME (FIG. 8).

Fractions of high purity were combined, diluted 6-fold in 50 mM Tris-HCl, 15 mM 2-ME and loaded onto a second anion exchange column Mono Q, Pharmacia (FIG. 9). The protein in fractions of highest purity was concentrated to 10-20 mg/ml in the elution buffer from the Mono Q 50 mM Tris-HCl pH 8.0, 15 mM 2-ME and 100-150 mM NaCl and used for crystallization trials (FIG. 9).

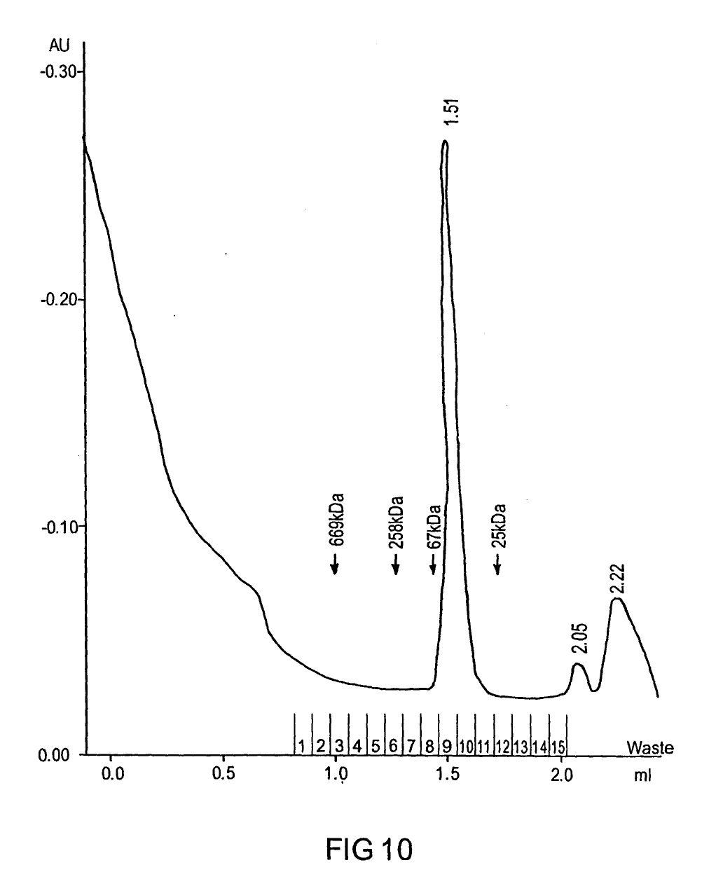

Analysis of the purified protein on size exclusion chromatography revealed that it eluted at a volume consistent with it being dimeric, assuming a protein of roughly spherical structure (FIG. 10). Under no circumstances were any lower or higher order species observed, even when the protein was left at room temperature overnight, suggesting that the protein was most likely a stable dimer.

### Example 2

### Crystallisation of Siah Domain Binding Site Fragment

Siah fragment was crystallized using the hanging drop vapour diffusion method with a precipitant solution of 100 mM MES pH 6.5, 20 mM CaCl₂ and 20% ethanol. Twenty-four well tissue culture plates were used for crystallisation. Drops consisted of 2 μl protein solution and 2 μl precipitant solution on a cover slide inverted over a 1 ml reservoir of precipitant. The chamber was sealed with vacuum grease to minimise evaporation. Crystals grew in 1-5 days at room temperature, attaining approximately 0.1×0.4 mm after 1-2 weeks. The addition of 50 μM zinc acetate has been found to improve the crystals.

### Example 3

### Binding of a 23mer Peptide to Siah Binding Domain

A twenty-three residue peptide from the protein PHYL (QQERTKLRPVAMVRPTVRVQPQL) was expressed as a fusion with glutathione-S-transferase (GST). The fusion protein was purified from E. coli extracts using affinity chromatography on glutathione-Sepharose and the protein was left on the resin. As a control, GST alone was expressed and purified in a similar manner.

For binding assays, 200 μl of Siah fragments (as fusions with the protein maltose binding protein, MBP) at approximately 0.5 mg/ml were mixed with 20 μl of glutathione-Sepharose resin (with GST or fusions) for 90min at 4° C. The resins were then pelleted, the supernatant discarded, and the resin washed 4 times in binding buffer (50 mM Tris HCl, pH8, 200 mM NaCl, 15 mM □-ME) before boiling in SDS-PAGE sample buffer for resolution on polyacrylamide gels. Visualisation of bound proteins was by Coomassie blue staining. As a control for the binding proteins, MBP alone was used.

Finally it is to be understood that various other modifications and/or alterations may be made without departing from the spirit of the present invention as outlined herein.

### References

Borden, K. L., and Freemont, P. S. (1996). The RING finger domain: a recent example of a sequence-structure family. Curr Opin Struct Biol 6, 395-401.
Bartlett, P. A. CAVEAT: A Program to Facilitate the Structure-Derived Design of Biologically Active Molecules. In Molecular Recognition in Chemical and Biological Problems. Special Pub., Royal Chem. Soc., 78 pp.182-196 (1989).
Baxter, C. A., Murray, C. W., Clark, D. E., Westhead, D. R., Eldridge, M. D. Flexible docking using tabu search and an empirical estimate of binding affinity. Proteins 33: 367-382, 1998.
Beddell, Drug development based on information regarding receptor structure. Chem. Soc. Reviews 1985, 279.
Blundell et al., Nature 1987 326 347.
Bohm H.-J., The Computer Program LUDI: A New Method for the De Novo Design of Enzyme Inhibitors. J. Comp. Aid. Molec. Design 6, pp.61-78 (1992).
Brunger, A. T., Acta Crystallogr. D. Biol Crystallogr 54: 905-21 (1988). Cohen, N. C. et al. Molecular Modeling Software and Methods for Medicinal Chemistry, J. Med. Chem., 33, pp.883-894 (1990).
DeWitte, R. S., Shakhnovich, E. I. SMoG: de novo design method based on simple fast and accurate free energy estimates. 1. Methodology and supporting evidence. J. Am. Chem. Soc. 118: 11733-11744, 1996.
De Witte, R. S., Ischenko, A. V., Shakhnovich, E. I. SMoG. de novo design method based on simple fast and accurate free energy estimates. 2. Case studies in molecular design. J. Am. Chem. Soc. 119:4608-4617,1997.
Eldridge, M. D., Murray, C. W., Auton, T. R., Paolini, G. V., Mee, R. P. Empirical scorint functions:l. The development of a fast empirical scoring function to estimate the binding affinity of ligands in receptor complexes. J. Comp. -Aided Mol. Des. 11: 425-445, 1997.
Fischer, P M, Krausz, E and Lane, D P. (2001) Cellular delivery of impermeable effector molecules in the form of conjugates with peptides capable of mediating membrane translocation. Bioconjugate Chemistry. 12, 825-841
Germani, A., Bruzzoni-Giovanelli, H., Fellous, A., Gisselbrecht, S., Varin-Blank, N., and Calvo, F. (2000). SIAH-1 interacts with alpha-tubulin and degrades the kinesin Kid by the proteasome pathway during mitosis.[In Process Citation]. Oncogene 19, 5997-6006.
Germani, A., Romero, F., Houlard, M., Camonis, J., Gisselbrecht, S., Fischer, S., and Varin-Blank, N. (1999). hSiah2 is a new Vav binding protein which inhibits Vav-mediated signaling pathways. Mol Cell Biol 19, 3798-807.
Goodford, J. Med. Chem. 1984 27 557;
Goodford, P. J. A Computational Procedure for Determining Energetically Favorable Binding Sites on Biologically Important Macromolecules. J. Med. Chem., 28, pp.849-857 (1985).
Goodsell, D. S. et al. Automated Docking of Substrates to Proteins by Simulated Annealing. Proteins: Structure, Function, and Genetics, 8, pp.195-202 (1990)
Hol, Angew. Chem. 1986 25 767
Hu, G., and Fearon, E. R. (1999). Siah-1 N-terminal RING domain is required for proteolysis function, and C-terminal sequences regulate oligomerization and binding to target proteins. Mol Cell Biol 19, 724-32.
Ishikawa, K., Nash, S. R., Nishimune, A., Neki, A., Kaneko, S., and Nakanishi, S. (1999). Competitive interaction of seven in absentia homolog-1A and Ca2+/calmodulin with the cytoplasmic tail of group 1 metabotropic glutamate receptors. Genes Cells 4, 381-90.
Karlin S., Zhu Z.-Y. and Karlin K. D. (1997) Proc. Natl. Acad. Sci. USA. 94, 14225-14230.
Kuntz, I. D. et al. A Geometric Approach to Macromolecule-Ligand Interactions. J. Mol. Biol. 161, pp.269-288 (1982)
Kuntz, I. D. Structure-based strategies for drug design and discovery. Science 257: 1078-1082, 1992.
Laroche Y., Storme V., De Meutter J., Messens J. and Lauwereys M. (1994). High-level secretion and very efficient isotopic labeling of tick anticoagulant peptide (TAP) expressed in the methylotrophic yeast, Pichia pastoris. Biotechnology 12: 1119-24.
Liew, C. K., Kowalski, K., Fox, A. H., Newton, A., Sharpe, B. K., Crossley, M., and Mackay, J. P. (2000). Solution Structures of Two CCHC Zinc Fingers from the FOG Family Protein U-Shaped that Mediate Protein-Protein Interactions. Structure Fold Des 8, 1157-1166.
Martin Y. C., 3D Database Searching in Drug Design. J. Med. Chem., 35, pp.2145-2154 (1992)
Matsuzawa, S., Takayama, S., Froesch, B. A., Zapata, J. M., and Reed, J. C. (1998). p53-inducible human homologue of Drosophila seven in absentia (Siah) inhibits cell growth: suppression by BAG-1. Embo J 17, 2736-47.
Meng, E. C. et al. J. Comp. Chem., 13, pp.505-524 (1992)
Miranker A. et al. Functionality Maps of Binding Sites: A Multiple Copy Simultaneous Search Method. Proteins: Structure, Function and Genetics, 11, pp.29-34 (1991)
Muegge, I., Martin, Y. C. A general and fast scoring function for proten-ligand interactios: A simplified potential approach. J. Med. Chem. 42:791-804, 1999.
Navia, M. A. et al. The Use of Structural Information in Drug Design, Current Opinions in Structural Biology, 2, pp.202-210 (1992)
Nishibata Y. et al., Tetrahedron, 47, p.8985 (1991)
Park, Y. C., Burkitt, V., Villa, A. R., Tong, L., and Wu, H. (1999). Structural basis for self-association and receptor recognition of human TRAF2. Nature 398, 533-8.
Park, Y. C., Ye, H., Hsia, C., Segal, D., Rich, R. L., Liou, H. C., Myszka, D. G., and Wu, H. (2000). A novel mechanism of TRAF signaling revealed by structural and functional analyses of the TRADD-TRAF2 interaction. Cell 101, 777-87.
Prigge S. T., Kolhekar A. S., Eipper B. A. and Mains R. E., (1997) Science, 278 1300.
Relaix, F., Wei, X., Li, W., Pan, J., Lin, Y., Bowtell, D. D., Sassoon, D. A., and Wu, X. (2000). Pw1/Peg3 is a potential cell death mediator and cooperates with Siah1a in p53-mediated apoptosis. Proc Natl Acad Sci USA 97, 2105-2110.
Schulman, B. A., Carrano, A. C., Jeffrey, P. D., Bowen, Z., Kinnucan, E. R., Finnin, M. S., Elledge, S. J., Harper, J. W., Pagano, M., and Pavletich, N. P. (2000). Insights into SCF ubiquitin ligases from the structure of the Skp1-Skp2 complex Nature 408, 381-6.
Schwabe, J. W., and Klug, A. (1994). Zinc mining for protein domains [news; comment). Nat Struct Biol 1, 345-9.
Sheridan and Venkataraghavan, Acc. Chem Res. 1987 20 322;
Smith, C L, Nawaz, Z and O'Malley, B W. (1997) Coactivator and corepressor regulation of the agonist/antagonist activity of the mixed antiestrogen, 4-hydroxytamoxifen. Mol. Endocrinology 11, 657-666
Stahl, M., Rarey, M. Detailed analysis of scoring functions for virtual screening. J. Med. Chem. 2001.
Stebbins, C. E., Kaelin, W. G., Jr., and Pavletich, N. P. (1999). Structure of the VHL-ElonginC-ElonginB complex: implications for VHL tumor suppressor function. Science 284, 455-61.
Takeuchi, M., Rothe, M., and Goeddel, D. V. (1996). Anatomy of TRAF2. Distinct domains for nuclear factor-kappaB activation and association with tumor necrosis factor signaling proteins. J Biol Chem 271, 19935-42.
Tang, A. H., Neufeld, T. P., Kwan, E., and Rubin, G. M. (1997). PHYL acts to down-regulate TTK88, a transcriptional repressor of neuronal cell fates, by a SINA-dependent mechanism. Cell 90, 459-67.
Tao, P. Lai, L. Protein ligand docking based on empirical method for binding affinity estimiation. J. Comp.-Aided Mol. Des. 15:429-446, 2001.
Verlinde C. L. M. J & Hol, W. G. J. Structure 1994, 2, 577.
Wang, R., Lui, L., Lai, L., Tang, Y. SCORE: A new empirical method for estimating the binding affinity of a protein-ligand complex. J. Mol. Model. 4: 379-394, 1998.
Wang, R., Wang, S. How does consensus scoring work for virtual library screening? An idealized computer experiment. J. Chem. Inf. Comput. Sci. 2001.
Zhang, J., Guenther, M. G., Carthew, R. W., and Lazar, M. A. (1998). Proteasomal regulation of nuclear receptor corepressor-mediated repression. Genes Dev 12, 1775-80.

Zheng, N., Wang, P., Jeffrey, P. D., and Pavletich, N. P. (2000). Structure of a c-Cbl-UbcH7 complex: RING domain function in ubiquitin-protein ligases. Cell 102, 533-9.

Diffraction data statistics

| Data set | Resolution, (Å) | Completeness, (%) | 1/ in the last resolution shell | R_{merge,} (%) | Multiplicity |
|---|---|---|---|---|---|
| Native | 20-2.6 | 99.8 | 4.0 | 6.3(38.8) | 8 |

MAD diffraction data statistics

| Data set | Wavelength, (Å) | Resolution, (Å) | Completeness, (%) | Multiplicity | R_{merge} |
|---|---|---|---|---|---|
| siah_peak | 1.28283 | 30-2.7 | 100.0 | 7.6 | 4.5(17.9) |
| siah_infl | 1.28336 | 30-2.7 | 100.0 | 14.0 | 6.1(34.5) |
| siah_remo1 | 1.24421 | 30-2.7 | 100.0 | 7.8 | 4.9(25.8) |
| siah_remo2 | 1.28616 | 30-2.7 | 100.0 | 14.0 | 5.2(27.6) |

Phasing statistics MAD as MIR (siah_infl as native)

| | Resolution, | Resolution_{ano,} | Phasing power | | R_{cullis} | | FOM | | |
|---|---|---|---|---|---|---|---|---|---|
| Derivative | (Å) | (Å) | acentric | centric | acentric | centric | acentric | centric | R_{cullis}_ano |
| siah_peak | 30-2.7 | 30-2.7 | 0.13 | 0.08 | 0.99 | 0.99 | 0.60 | 0.54 | 0.73 |
| siah_remo1 | 30-2.7 | 30-2.7 | 1.43 | 0.95 | 0.77 | 0.71 | | | 0.74 |
| siah_remo2 | 30-2.7 | 30-2.7 | 0.89 | 0.55 | 0.88 | 0.88 | | | 0.97 |

Final refinement statistics

| Data set | Native |
|---|---|
| Resolution | 20-2.6 Å |
| R-factor/R-free | 22.8/27.7% |
| R.m.s.d. bond lengths | 0.007 Å |
| R.m.s.d. bond angles | 1.25° |
| R.m.s.d. dihedral angles | 22.7° |
| R.m.s.d. bonded B's (m-c) | 1.34 Å² |
| R.m.s.d. bonded B's (s-c) | 2.00 Å² |

1-83. (canceled)

84. A binding domain of Siah comprising an amino acid residue found in the regions defined by Pro97 to Cys282 of Siah1 a and equivalent residues.

85. A binding domain according to claim 84 comprising a region selected from the group consisting of Leu158 to Cys282, Cys 130 to Glu194, Glu113 to Glu269, Glu197 to Ser262, Pro97 to Val192, and Cys 105 to Phe123 of Siah1 a and equivalent residues.

86. A binding domain according to claim 84 comprising amino acid residues selected from the group consisting of Gly132, Glu194, and Trp178 of Siah1 a or equivalent residues

87. A binding domain according to claim 84 comprising the amino acid residues Gly160, Cys184, Phe185, Phe187, Phe221, Met281, and Glyl 86 of Siah 1 a or equivalent residues.

88. A binding domain according to claim 84 comprising at least one amino acid residue selected from the group consisting of Arg233, Thr235, Glu237, His230, Asn228, Giu226, Arg224, Ala222, Asn276, Thr278, Ser280, Met281, Cys282, Phe185, Thr168, Leu166, Val 164, Asp162, Glu161, Gly160, Leu158, Arg231 and Cys184 of Siah1a or equivalent residues.

89. A binding domain according to claim 84 comprising at least one amino acid residue selected from the group consisting of Trp178, Lys153, Thr168, Thr156, Phe165, VaI179, Ser154, Gly174, Cys130, Ser134, Cys135, His152, Asp177, Asp169, Leu172, Pro173, Glu194, Pro131 and Leu166 of Siah1a or equivalent residues.

90. A binding domain according to claim 84 comprising at least one amino acid residue selected from the group consisting of Arg231, Arg232, Glu219, Lys216, Arg241, Giu245, Arg215, Asp255, Asn253, Leu266, Glu269, His230, Gln265, Met252, Thr249, Leu120, Glu119, Glu116, Glu113, Asn270 and Ser262 of Siah1a or equivalent residues.

91. A binding domain according to claim 84 comprising at least one amino acid residue selected from the group consisting of Asp200, His202, Tyr199, Lys198, Gln204, Met252, Ser262, Asp200, Gln196, Asp260, Gly201, Thr261 and Gln203 of Siah1 a or equivalent residues.

92. A binding domain according to claim 84 wherein the domain comprises at least one amino acid residue from Pro97 to VaI192 of Siah1a or equivalent residues.

93. A binding domain according to claim 84 comprising at least one amino acid residue selected from the group consisting of Lys114, Pro97, Glu118, His111, Phe96, Lys99, Tyr100, Arg124, Glu119, Ala115 and Val192 of Siah1a or equivalent residues.

94. A binding domain according to claim 84 comprising at least one amino acid residue from Cys121, His117, Cys105, Ile107, Leu109, Phe123, Leu120 and Glu122 of Siah1a or equivalent residues.

95. A binding domain according to claim 84 wherein the domain is capable of being occupied by 2-ME in the crystal structure.

96. A dimerisation domain of Siah comprising the amino acid residues selected from the group consisting of Arg241 monomer A, Asp255 monomer A, and Arg232 monomer B of Siah1 a and equivalent residues.

97. A dimerisation domain according to claim 96 comprising the amino acid residues Glu219OE1-Arg231 NH2, Arg232NE-Asp255OD1, Ser2620G-Ser2540G, Gln204, Asp260, Asp200 monomer A, His202A, His 202B and His150B-symmetry related of Siah1a or equivalent residues.

98. A zinc-binding domain of Siah or portion thereof of a Siah protein comprising a R-strand, 1i-strand, a-helix structure comprising an amino acid residue selected from the group including Cys98, Cys105, His117, Cys121, Cys128, Cys135, His147, His 150, and His152 of Siah1a or equivalent residues.

99. A zinc-binding domain of Siah or a portion thereof comprising the amino acid residues His230 monomer A, Glu269 monomer A, His230 monomer B, and GIu269 monomer B of Siah1 a or equivalent residues.

100. A ligand capable of binding to Siah, the ligand comprising the amino acid sequence VXP (SEQ ID NO:6).

101. A ligand capable of binding to Siah, the ligand comprising the amino acid sequence PXAXVXP (SEQ ID NO:3).

102. A ligand capable of binding to Siah, the ligand comprising the amino acid sequence RPVAXVXPXXR (SEQ ID NO: 1).

103. A method of identifying a compound which is capable of acting as an agonist or antagonist of binding to Siah, the method comprising the step of identifying a compound that has a conformation and polarity such that it interacts with an amino acid residue selected from the group consisting of: Arg233, Thr235, Glu237, His230, Asn228, Glu226, Arg224, Ala222, Asn276, Thr278, Ser280, Met281, Cys282, Phe185, Thr168, Leu166, VaI164, Asp162, Glu161, Gly160, Leu158, Arg231, Cys184, Trp178, Lys153, Thr168, Thr156, Phe165, VaI179, Ser154, Gly174, Cys130, Ser134, Cys135, His152, Asp177, Asp169, Leu172, Pro173, Glu194, Pro131, Leu166, Arg231, Arg232, Glu219, Lys216, Arg241, Glu245, Arg215, Asp255, Asn253, Leu266, Glu269, His230, Gln265, Met252, Thr249, Leu120, Glu119, Glu116, Glu113, Asn270, Ser262; Asp200, His202, Tyr199, Lys198, Gln204, Met252, Ser262, Asp200, Gln196, Asp260, Gly201, Thr261, Gln203, Lys114, Pro97, Glu118, His111, Phe96, Lys99, Tyr100, Arg124, Glu119, Ala115, Val192; Cys121, His117, Cys105, Ile107, Leu109, Phe123, Leu120 and Glu122; of Siah1a or equivalent residues.

104. A method according to claim 103 wherein the compound interacts with at least two residues, or at least three residues, or at least 4 residues.

105. A method according to claim 103 wherein the method is implemented in hardware or software, or a combination of both.

106. A method according to claim 103 wherein the method is carried out us an in vivo or in vitro assay selected from the group consisting of a binding assay, a competition binding assay, a crystallisation assay, a biosensor assay and X-Ray crystallography.

107. A computer-assisted method of identifying compounds potentially able to bind to the binding domain of Siah using a programmed computer the method comprising the steps of: (a) inputting into the programmed computer data comprising the atomic coordinates of the Siah binding domain, according to FIG. 21, corresponding to a binding site defined by amino acid residues Arg233, Thr 235, Glu237, His230, Asn228, Glu226, Arg224, Ala222, Asn276, Thr278, Ser280, Met281, Cys282, Phe185, Thr168, Leu166, Val164, Asp162, Glu161, Gly160, Leu158, Arg231, Cys184, Trp178, Lys153, Thr168, Thr156, Phe165, Val179, Ser154, Gly174, Cys130, Ser134, Cys135, His152, Asp177, Asp169, Leu172, Pro173, Glu194, Pro131, Leu166, Arg231, Arg232, Glu219, Lys216, Arg241, Glu245, Arg215, Asp255, Asn253, Leu266, Glu269, His230, Gln265, Met252, Thr249, Leu120, Glu119, Glu116, Glu113, Asn270, Ser262, Asp200, His202, Tyr199, Lys198, Gln204, Met252, Ser262, Asp200, Gln196, Asp260, Gly201, Thr261 Gln203, Lys114, Pro97, Glu118, His111, Phe96, Lys99, Tyr100, Arg124, Glu119, Ala115 Val192, Cys121, His117, Cys105, Ile 107, Leu 109, Phe123, Leu 120, Glu 122 of Siah 1 a or equivalent residues; (b) generating, using computer methods, a set of atomic coordinates of a structure that possesses stereochemical complementarity to the atomic coordinates defined in (a) or a subset thereof, thereby generating a criteria data set; (c) comparing, using the processor, the criteria data set to a computer database of chemical structures; (d) selecting from the database, using computer methods, chemical structures which are similar to a portion of the criteria data set; and (e) outputting the selected chemical structures which are similar to a portion of the criteria data set.

108. A method according to claim 107 wherein the method further comprises the step of obtaining a compound with a chemical structure selected in steps (d) and (e), and testing the compound for the ability to bind to Siah.

109. A computer or a software component thereof for producing a three dimensional representation of a molecule or molecular complex, which comprises a three-dimensional representation of a homologue of the molecule or molecular complex, in which the homologue comprises a domain that has a root mean square deviation from the backbone atoms of the amino acids of not more than 1.5 A, in which the computer comprises: (a) a machine-readable data storage medium comprising a data storage material encoded with machine-readable data, wherein the data comprises the atomic coordinates, according to FIG. 21, of: Arg233, Thr235, Glu237, His230, Asn228, Glu226, Arg224, Ala222, Asn276, Thr278, Ser280, Met281, Cys282, Phe185, Thr168, Leu166, Val164, Asp162, Glu161, Gly160, Leu158, Arg231, Cys184, Trp178, Lys153, Thr168, Thr156, Phe165, VaI179, Ser154, Gly174, Cys130, Ser134, Cys135, His152, Asp177, Asp169, Leu172, Pro173, Glu194, Pro131, Leu166, Arg231, Arg232, Glu219, Lys216, Arg241, Glu245, Arg215, Asp255, Asn253, Leu266, Glu269, His230, Gln265, Met252, Thr249, Leu120, Glu119, Glu116, Giul13, Asn270, Ser262, Asp200, His202, Tyr199, Lys198, Gln204, Met252, Ser262, Asp200, Gln196, Asp260, Gly201, Thr261 GIn203, Lys114, Pro97, Glu118, His111, Phe96, Lys99, Tyr100, Arg124, Glu119, Ala115, Val192, Cys121, His117, Cys105, Ile107, Leu109, Phe123, Leu120, Glu 122 of Siah 1a or equivalent residues; (b) a working memory for storing instructions for processing the machine-readable data; (c) a central-processing unit coupled to the working memory and to the machine-readable data storage medium for processing the machine-readable data into the three-dimensional representation; and (d) a display coupled to the central-processing unit for displaying the three-dimensional representation.

110. A method according to claim 109 wherein the three-dimensional representation is of a molecule or molecular complex defined by the set of atomic coordinates set out in FIG. 21, or wherein the three-dimensional representation is of a homologue of the molecule or molecular complex, the homologue having a root mean square deviation from the backbone atoms of the amino acids of not more than 1.5 A.

111. A computer or a software component thereof for determining at least a portion of the structure coordinates corresponding to a three-dimensional structure of a molecule or molecular complex, in which the computer comprises: (a) a machine-readable data storage medium comprising a data storage material encoded with machine-readable data, in which the data comprises at least a portion of the atomic coordinates according to FIG. 21; (b) a machine-readable data storage medium comprising a data storage material encoded with machine-readable data, wherein the data comprise crystallographic data of the molecule or molecular complex ; (c) a working memory for storing instructions for processing the machine-readable data of (a) and (b); (d) a central-processing unit coupled to the working memory and to the machine-readable data storage medium of (a) and (b) for performing a transformation of the machine readable data of (a) and for processing the machine-readable data of (b) into structure coordinates; and (e) a display coupled to the central-processing unit for displaying the structure coordinates of the molecule or molecular complex.

112. An antagonist or agonist of the binding of substrate and/or co-factor and/or interactor and/or ligands to a binding domain of Siah identified by a method according to claims 103.

113. An antagonist or agonist according to claim 112 having a conformation and polarity such that it binds at least one amino acid residue selected from the group consisting of: Arg233, Thr235, Glu237, His230, Asn228, Glu226, Arg224, Ala222, Asn276, Thr278, Ser280, Met281, Cys282, Phe185, Thr168, Leu166, Val164, Asp162, Glu161, Gly160, Leu158, Arg231, Cys184, Trp178, Lys153, Thr168, Thr156, Phe165, VaI179, Ser154, Gly174, Cys130, Ser134, Cys135, His152, Asp177, Asp169, Leu172, Pro173, Glu194, Pro131, Leu166, Arg231, Arg232, Glu219, Lys216, Arg241, Glu245, Arg215, Asp255, Asn253, Leu266, Glu269, His230, Gln265, Met252, Thr249, Leu120, Glu119, Glu116, Glu113, Asn270, Ser262, Asp200, His202, Tyr199, Lys198, Gln204, Met252, Ser262, Asp200, Gln196, Asp260, Gly201, Thr261, Gln203, Lys114, Pro97, Glu118, His111, Phe96, Lys99, Tyr100, Arg124, Glu119, Ala115, VaI192, Cys121, His117, Cys105, Ile107, Leu109, Phe123, Leu120 and Glu122 ; of Siah 1a or equivalent residues.

114. An antagonist or agonist according to claim 113 capable of binding with at least two amino acid residues, or at least three amino acid residues, or at least four amino acid residues.

115. An antagonist or agonist according to claim 113 wherein the agonist has an affinity constant of <1 uM, or preferably <1 nM for Siah.

116. A composition comprising a ligand according to claim 100 and a pharmaceutically-acceptable carrier.

117. A method of treating or preventing a Siah-related condition, the method comprising administering to a subject in need thereof an effective amount of a composition according to claim 116.

118. A method according to claim 117 wherein the condition is selected from the group including cancer, inflammation, infertility, a pathological immune response, an apoptosis-related disease, a protein degradation-related disease, and an NFκB signalling-related disease.

119. A crystalline form of Siah.

120. A crystalline form of Siah having at least one atomic co-ordinate within 0.5 angstrom of an atomic co-ordinate defined in FIG. 21.

121. A crystalline form the Siah dimer comprising 4 molecules of 2-ME, 6 zinc ions and 65 water molecules.

122. A method of producing a crystal of Siah or a fragment thereof comprising using partially oxidised 2-ME.

123. A three dimensional model of a Siah binding domain or homologue thereof defined by the set of atomic co-ordinates set out in FIG. 21, the homologue having a root mean square deviation from the backbone atoms of the amino acids of not more than 1.5 Angstroems.

124. Use of the model according to claim 123 for rational drug decision.
